SEQUENCE LISTING (46 sequences)

SEQ ID NO: 1 - DNA, 3579 bp, Homo sapiens
atgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtga

SEQ ID NO: 2 - DNA, 810 bp, Adeno-associated virus
gtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgccc

SEQ ID NO: 3 - DNA, 8562 bp, Adeno-associated virus
gcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 4 - DNA, 564 bp, Adeno-associated virus
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 5 - DNA, 8409 bp, Adeno-associated virus
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagacagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 6 - DNA, 8611 bp, Adeno-associated virus
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 7 - DNA, 792 bp, Adeno-associated virus
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 8 - DNA, 8629 bp, Kanamycin Plasmid
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagct

SEQ ID NO: 9 - DNA, 4977 bp, Artificial Sequence
  Kanamycin Cassatte
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 10 - protein, 142 aa, Artificial Sequence
  Synthetic Polypeptide
MDWLWNLLFLMAAAQSAQTQIQLVQSGPELRKPGETVKISCKASGYSFTNYGMNWVKQTPGKDLKWMGWINTYTGEPTYADDFKGRFAFSLEASANTAYLQINDLKNEDMATYFCARGVAHYSDSRFAFDYWGQGTTLTVPS

SEQ ID NO: 11 - protein, 128 aa, Artificial Sequence
  Synthetic Polypeptide
MHFQVQIFSFLLISASVIMSRGQIVLTQSPAIMSASPGEKVTITCSVSSSVSYMHWFQQKPGTSPKLWIYYTSNLASGVPGRFSGSGSGTSYSLTISRMEAEDAATYYCQQRSSYPFTFGSGTKLEIK

SEQ ID NO: 12 - protein, 140 aa, Artificial Sequence
  Synthetic Polypeptide
MDWLWNLLFLMAAAQSAQTQIQLVQSGPELKKPGETVKISCKAAGYTFTDYGMNWVKQAPGEGLKWMGWINTNTGEPTYGDDFKGRFAFSLEASASTAHLQINNLKNDDMAIYFCARGNAHPGGSAFVYWGQGTLVTVSA

SEQ ID NO: 13 - protein, 128 aa, Artificial Sequence
  Synthetic Polypeptide
MHFQVQIFSFLLISASVIMSRGQIVLTQSPAIMSASPGESVTITCSASSGVTYMHWFQQKPGTSPKNWIYRTSNLASGVPARFSGSGSGTSYSLTISRMEAEDAATYYCQQRSSYPFTFGSGTKLEIK

SEQ ID NO: 14 - protein, 123 aa, Artificial Sequence
  Synthetic Polypeptide
QVKLEESGPELKKPGETVKISCKASGYTFTNYGMNWVKQAPGKGLKWMGWINTYTGEPTYADDFKGRFAFSLETSARKVYLQINNLKNEDMATYFCARGSYYYDSSPAWFAYWGQGTLVTVSA

SEQ ID NO: 15 - protein, 107 aa, Artificial Sequence
  Synthetic Polypeptide
QIVLTQSPAIMSASPGEKVTITCSASSSVSYMHWFQQKPGTSPKLWIYSTSNLASGVPARFSGSGSGTSYSLTISRMEAEDAATYYCQQRSTYPFTFGSGTKLEIKR

SEQ ID NO: 16 - protein, 121 aa, Artificial Sequence
  Synthetic Polypeptide
QVKLQESGPELKKPGETVKISCKASGYTFTKYGMNWVKQAPGEGLKWMGWINTYTGEPTYADDFKGRFAFSLKTSASTAYLQINNLKNEGTTTYFCARGVDSSGYGAFAYWGQGTLVTVSA

SEQ ID NO: 17 - protein, 107 aa, Artificial Sequence
  Synthetic Polypeptide
QIVLTQSPAIMSASPGEKVTITCSASSSVSYMHWFQQKPGTSPKLWIYSTSNLASGVPARFSGSGSGTSYSLTISRMEAEDAATYYCQQRSFYPFTFGSGTKLEIKR

SEQ ID NO: 18 - protein, 122 aa, Artificial Sequence
  Synthetic Polypeptide
EVQLQESGSDLKKPGETVKISCKASGYTFTNYGMNWVKQAPGKGLKWMGWINTYTGEPTYADDFKGRFAFSLETSASTAFLQINNLKYEDTGTYFCTRGTSTMISTFAFVYWGQGTLVTVSA

SEQ ID NO: 19 - protein, 107 aa, Artificial Sequence
  Synthetic Polypeptide
QIVLTQSPAIMSASPGEKVTITCSASSSVRYMHWFQQKPGTSPKVWIYSTSNLASGVPARFSGSGSGTSYSLTISRMEAEDAATYYCQQRTYYPFTFGSGTKLEIKR

SEQ ID NO: 20 - protein, 5 aa, Artificial Sequence
  Synthetic Polypeptide
NYGMN

SEQ ID NO: 21 - protein, 9 aa, Artificial Sequence
  Synthetic Polypeptide
YTFTNYGMN

SEQ ID NO: 22 - protein, 5 aa, Artificial Sequence
  Synthetic Polypeptide
DYGMN

SEQ ID NO: 23 - protein, 9 aa, Artificial Sequence
  Synthetic Polypeptide
YTFTKYGMN

SEQ ID NO: 24 - protein, 17 aa, Artificial Sequence
  Synthetic Polypeptide
WINTYTGEPTYADDFKG

SEQ ID NO: 25 - protein, 17 aa, Artificial Sequence
  Synthetic Polypeptide
WINTNTGEPTYGDDFKG

SEQ ID NO: 26 - protein, 14 aa, Artificial Sequence
  Synthetic Polypeptide
WMGWINTYTGEPTY

SEQ ID NO: 27 - protein, 14 aa, Artificial Sequence
  Synthetic Polypeptide
GVAHYSDSRFAFDY

SEQ ID NO: 28 - protein, 12 aa, Artificial Sequence
  Synthetic Polypeptide
GNAHPGGSAFVY

SEQ ID NO: 29 - protein, 15 aa, Artificial Sequence
  Synthetic Polypeptide
RGSYYYDSSPAWFAY

SEQ ID NO: 30 - protein, 13 aa, Artificial Sequence
  Synthetic Polypeptide
RGVDSSGYGAFAY

SEQ ID NO: 31 - protein, 15 aa, Artificial Sequence
  Synthetic Polypeptide
TRGTSTMISTFAFVY

SEQ ID NO: 32 - protein, 10 aa, Artificial Sequence
  Synthetic Polypeptide
SVSSSVSYMH

SEQ ID NO: 33 - protein, 10 aa, Artificial Sequence
  Synthetic Polypeptide
SASSGVTYMH

SEQ ID NO: 34 - protein, 7 aa, Artificial Sequence
  Synthetic Polypeptide
SSVSYMH

SEQ ID NO: 35 - protein, 7 aa, Artificial Sequence
  Synthetic Polypeptide
SSVRYMH

SEQ ID NO: 36 - protein, 7 aa, Artificial Sequence
  Synthetic Polypeptide
YTSNLAS

SEQ ID NO: 37 - protein, 7 aa, Artificial Sequence
  Synthetic Polypeptide
RTSNLAS

SEQ ID NO: 38 - protein, 11 aa, Artificial Sequence
  Synthetic Polypeptide
LWIYSTSNLAS

SEQ ID NO: 39 - protein, 11 aa, Artificial Sequence
  Synthetic Polypeptide
VWIYSTSNLAS

SEQ ID NO: 40 - protein, 9 aa, Artificial Sequence
  Synthetic Polypeptide
QQRSSYPFT

SEQ ID NO: 41 - protein, 8 aa, Artificial Sequence
  Synthetic Polypeptide
QQRSTYPF

SEQ ID NO: 42 - protein, 8 aa, Artificial Sequence
  Synthetic Polypeptide
QQRSFYPF

SEQ ID NO: 43 - protein, 8 aa, Artificial Sequence
  Synthetic Polypeptide
QQRTYYPF

SEQ ID NO: 44 - DNA, 2303 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggggttaaccaattggcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccaccatggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaaaagcttggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 45 - DNA, 957 bp, Homo sapiens
  misc_feature: Beta-sarcoglycan
atggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaa

SEQ ID NO: 46 - protein, 318 aa, Homo sapiens
  MISC_FEATURE: Beta-sarcoglycan
MAAAAAAAAEQQSSNGPVKKSMREKAVERRSVNKEHNSNFKAGYIPIDEDRLHKTGLRGRKGNLAICVIILLFILAVINLIITLVIWAVIRIGPNGCDSMEFHESGLLRFKQVSDMGVIHPLYKSTVGGRRNENLVITGNNQPIVFQQGTTKLSVENNKTSITSDIGMQFFDPRTQNILFSTDYETHEFHLPSGVKSLNVQKASTERITSNATSDLNIKVDGRAIVRGNEGVFIMGKTIEFHMGGNMELKAENSIILNGSVMVSTTRLPSSSSGDQLGSGDWVRYKLCMCADGTLFKVQVTSQNMGCQISDNPCGNTH

(19) United States
(12) Patent Application Publication
(10) Pub. No.: US 2021/0393801 A1
(43) Pub. Date: Dec. 23, 2021

(54) Adeno-Associated Virus Vector Delivery for Muscular Dystrophies

(71) Applicants: RESEARCH INSTITUTE AT NATIONWIDE CHILDREN'S HOSPITAL, Columbus, OH (US); SAREPTA THERAPEUTICS INC., Cambridge, MA (US)

(72) Inventors: Louise Rodino-Klapac, Dublin, OH (US); Jerry R. Mendell, Columbus, OH (US); Ellyn Peterson, Dublin, OH (US); Rachael Potter, Dublin, OH (US); Danielle Griffin, Dublin, OH (US)

(21) Appl. No.: 17/348,515

(22) Filed: Jun. 15, 2021

Related U.S. Application Data
(60) Provisional application No. 63/188,266, filed on May 13, 2021.
(60) Provisional application No. 63/160,376, filed on Mar. 12, 2021.
(60) Provisional application No. 63/083,953, filed on Sep. 27, 2020.
(60) Provisional application No. 63/039,252, filed on Jun. 15, 2020.

(51) Int. Cl. A61K 48/00; C12N 7/00; A61K 45/06; C07K 16/28; A61P 21/00

(52) U.S. Cl. CPC A61K 48/005; C12N 7/00; A61P 21/00; C07K 16/2887; A61K 45/06

(57) ABSTRACT

The disclosure provides method of treating muscular dystrophy in a subject in need comprising administering a gene therapy vector, such as adeno-associated virus (AAV) vector, expressing a miniaturized human micro-dystrophin gene in combination with a step of suppressing the subject's immune system.

This application claims priority benefit to U.S. Provisional Application No. 63/039,252, filed Jun. 15, 2020, U.S. Provisional Application No. 63/083,953, filed Sep. 27, 2020, U.S. Provisional Application No. 63/160,376, filed Mar. 12, 2021 and U.S. Provisional Application No. 63/188,266, filed May 13, 2021, each of which is incorporated by reference herein in its entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 55714_Seqlisting.txt; Size: 83,380 bytes, created; Jun. 7, 2021.

### FIELD

The disclosure provides method of treating a disorder, such as muscular dystrophy, in a subject in need comprising administering a gene therapy vector, such as adeno-associated virus (AAV) vector, expressing a transgene of interest such as miniaturized human micro-dystrophin gene or β-sarcoglycan gene, in combination with a step of suppressing the subject's immune system.

### BACKGROUND

The importance of muscle mass and strength for daily activities, such as locomotion and breathing, and for whole body metabolism is unequivocal. Deficits in muscle function produce muscular dystrophies (MDs) that are characterized by muscle weakness and wasting and have serious impacts on quality of life. The most well-characterized MDs result from mutations in genes encoding members of the dystrophin-associated protein complex (DAPC). These MDs result from membrane fragility associated with the loss of sarcolemmal-cytoskeleton tethering by the DAPC. Duchenne Muscular Dystrophy (DMD) is one of the most devastating muscle disease affecting 1 in 5000 newborn males.

DMD is caused by mutations in the DMD gene leading to reductions in mRNA and the absence of dystrophin, a 427 kD sarcolemmal protein associated with the dystrophin-associated protein complex (DAPC) (Hoffman et al., Cell 51(6):919-28, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction and protect against contraction-induced damage. With dystrophin loss, membrane fragility results in sarcolemmal tears and an influx of calcium, triggering calcium-activated proteases and segmental fiber necrosis (Straub et al., Curr Opin. Neurol. 10(2): 168-75, 1997). This uncontrolled cycle of muscle degeneration and regeneration ultimately exhausts the muscle stem cell population (Sacco et al., Cell, 2010. 143(7): p. 1059-71; Wallace et al., Annu Rev Physiol, 2009. 71: p. 37-57), resulting in progressive muscle weakness, endomysial inflammation, and fibrotic scarring.

Without membrane stabilization from dystrophin or a micro-dystrophin, DMD will manifest uncontrolled cycles of tissue injury and repair ultimately replace lost muscle fibers with fibrotic scar tissue through connective tissue proliferation. Fibrosis is characterized by the excessive deposits of ECM matrix proteins, including collagen and elastin. ECM proteins are primarily produced from cytokines such as TGFβ that is released by activated fibroblasts responding to stress and inflammation. Although the primary pathological feature of DMD is myofiber degeneration and necrosis, fibrosis as a pathological consequence has equal repercussions. The over-production of fibrotic tissue restricts muscle regeneration and contributes to progressive muscle weakness in the DMD patient. In one study, the presence of fibrosis on initial DMD muscle biopsies was highly correlated with poor motor outcome at a 10-year follow-up (Desguerre et al., J Neuropathol Exp Neurol, 2009. 68(7): p. 762-7). These results point to fibrosis as a major contributor to DMD muscle dysfunction and highlight the need for early intervention prior to overt fibrosis.

Another group of MDs is the limb girdle group (LGMD) of MDs. LGMDs are rare conditions and they present differently in different people with respect to age of onset, areas of muscle weakness, heart and respiratory involvement, rate of progression and severity. LGMDs can begin in childhood, adolescence, young adulthood or even later. Both genders are affected equally. LGMDs cause weakness in the shoulder and pelvic girdle, with nearby muscles in the upper legs and arms sometimes also weakening with time. Weakness of the legs often appears before that of the arms. Facial muscles are usually unaffected. As the condition progresses, people can have problems with walking and may need to use a wheelchair over time. The involvement of shoulder and arm muscles can lead to difficulty in raising arms over head and in lifting objects. In some types of LGMD, the heart and breathing muscles may be involved.

There are at least nineteen forms of LGMD, and the forms are classified by their associated genetic defects.

| Type | Pattern of Inheritance | Gene or Chromosome |
|---|---|---|
| LGMD1A | Autosomal dominant | Myotilin gene |
| LGMD1B | Autosomal dominant | Lamin A/C gene |
| LGMD1C | Autosomal dominant | Caveolin gene |
| LGMD1D | Autosomal dominant | Chromosome 7 |
| LGMDlE | Autosomal dominant | Desmin gene |
| LGMD1F | Autosomal dominant | Chromosome 7 |
| LGMD1G | Autosomal dominant | Chromosome 4 |
| LGMD2A | Autosomal recessive | Calpain-3 gene |
| LGMD2B | Autosomal recessive | Dysferlin gene |
| LGMD2C | Autosomal recessive | Gamma-sarcoglycan gene |
| LGMD2D | Autosomal recessive | Alpha-sarcoglycan gene |
| LGMD2E | Autosomal recessive | Beta-sarcoglycan gene |
| LGMD2F | Autosomal recessive | Delta-sarcoglycan gene |
| LGMD2G | Autosomal recessive | Telethonin gene |
| LGMD2H | Autosomal recessive | TRIM32 |
| LGMD2I | Autosomal recessive | FKRP gene |
| LGMD2J | Autosomal recessive | Titin gene |
| LGMD2K | Autosomal recessive | POMT1 gene |
| LGMD2L | Autosomal recessive | Fukutin gene |

Specialized tests for LGMD are now available through a national scheme for diagnosis, the National Commissioning Group (NCG).

As gene therapies for treating muscular dystrophy, such as DMD or LGMD, are developed, there is a need for optimizing these therapies and to evaluate the effects of immunosuppression on the expression of the micro-dystrophin transgene by the optimized gene therapy vectors.

### SUMMARY OF INVENTION

The present disclosure is directed to gene therapy vectors, e.g. AAV, expressing a transgene of interest to skeletal muscles to treat a muscular dystrophy in combination with administration of immunosuppressants. In addition, the present disclosure includes methods of re-dosing a subject with the AAV gene therapy, wherein the subject's plasma is subjected to therapeutic plasma exchange (TPE) to remove AAV antibodies produced in response to the first dose of AAV gene therapy.

The present disclosure is directed to gene therapy vectors, e.g. AAV, expressing the micro-dystrophin gene to skeletal muscles including diaphragm and cardiac muscle to protect muscle fibers from injury, increase muscle strength and reduce and/or prevent fibrosis in combination with administration of immunosuppressants. In addition, the present disclosure includes methods of re-dosing a subject with the AAV gene therapy, wherein the subject's plasma is subjected to therapeutic plasma exchange (TPE) to antibodies the subject has remove AAV antibodies produced in response to the first dose of AAV gene therapy.

In addition, the disclosure is directed to gene therapy vectors, e.g. AAV, expressing the β-sarcoglycan gene to skeletal muscles including diaphragm and cardiac muscle in combination with administration of immunosuppressants. In addition, the present disclosure includes methods of re-dosing a subject with the AAV gene therapy, wherein the subject's plasma is subjected to therapeutic plasma exchange (TPE) to antibodies the subject has remove AAV antibodies produced in response to the first dose of AAV gene therapy.

The disclosure provides for combination therapies and approaches for increasing muscular force and/or increasing muscle mass using gene therapy vectors to deliver micro-dystrophin to address the gene defect observed in DMD. In particular, the present disclosure provides a study to demonstrate gene expression of systemic delivery of rAAVrh74.MHCK7.micro-dystrophin in the non-human primate model using different immunosuppressing regimens of which duration, dose and type of immunosuppression is altered. The present disclosure also provides a study to demonstrate micro-dystrophin transgene expression after the use of TPE to remove AAV virus antibodies from previously dosed non-human primates and systemically re-dose primates using rAAVrh74.MHCK7.micro-dystrophin.

The disclosure also provides for an approach for treating human subjects suffering from DMD which anti-AAVrh.74 antibodies prior to administration of rAAVrh74.MHCK7.micro-dystrophin, and the subject is subjected to multiple sessions of TPE prior to administration of the rAAVrh74.MHCK7.micro-dystrophin.

The disclosure provides for nucleic acid molecules comprising the nucleotide sequence of SEQ ID NO: 3, 8 or 9. The disclosure also provides for rAAV comprising the nucleic acid sequence of SEQ ID NO: 9 or nucleotides 1-4977 of SEQ ID NO: 8 or nucleotides 55-5021 of SEQ ID NO: 3, and rAAV particles comprising the nucleic acid sequence of SEQ ID NO: 9 or nucleotides 1-4977 of SEQ ID NO: 8 or nucleotides 55-5021 of SEQ ID NO: 3.

Another aspect of the disclosure provides for compositions comprising a nucleic acid molecule comprising the nucleotide sequence of SEQ ID NO: 3, 8 or 9, rAAV comprising the nucleic acid sequence of SEQ ID NO: 9 or nucleotides 1-4977 of SEQ ID NO: 8 or nucleotides 55-5021 of SEQ ID NO: 3, and rAAV particles comprising the nucleic acid sequence of SEQ ID NO: 9 or nucleotides 1-4977 of SEQ ID NO: 8 or nucleotides 55-5021 of SEQ ID NO: 3. Any of the methods disclosed herein may be carried out with these compositions.

In addition, the disclosure provide for a composition comprising a nucleic acid comprising the rAAV genome of one of the following AAVrh.74.tMCK.CAP N3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05.

The disclosure provides for methods of treating a muscular dystrophy in a human subject in need thereof comprising the step of comprising the step of administering a recombinant adeno-virus associated (rAAV) rAAV.MHCK7.microdystrophin and an anti-inflammatory steroid. In addition, the disclosure provides for use of a combination therapy comprising a recombinant adeno-virus associated (rAAV) rAAV.MHCK7.microdystrophin and an anti-inflammatory steroid for the preparation of a medicament for treating a muscular dystrophy in a human subject in need thereof, such that rAAV and the anti-inflammatory steroid of the medicament are co-administered separately, such as the rAAV and anti-inflammatory steroid are co-administered simultaneously or sequentially. The disclosure also provides for a combination therapy for treating a muscular dystrophy wherein the therapy comprises a recombinant adeno-virus associated (rAAV) rAAV.MHCK7.microdystrophin and an anti-inflammatory steroid, wherein rAAV and the anti-inflammatory steroid are co-administered separately, such as the rAAV and anti-inflammatory steroid are co-administered simultaneously or sequentially. For example, the muscular dystrophy is DMD or Becker's muscular dystrophy. For example, the anti-inflammatory steroid is a glucocorticoid. In some embodiments, the anti-inflammatory steroid is prednisone, prednisolone, betamethasone, dexamethasone, hydrocortisone, methylprednisolone or deflazacort. In some embodiments, the anti-inflammatory steroid is administered orally. The anti-inflammatory steroid may be administered both prior to and after administration of the rAAV. Alternatively, the anti-inflammatory steroid is administered only prior to or only after administration of the rAAV.

In addition, the disclosure provides for a method of treating a Limb Girdle Muscular Dystrophy in a human subject in need thereof comprising the step of administering a recombinant adeno-virus associated (rAAV) and an anti-inflammatory steroid, wherein the rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05. In addition, the disclosure provides for use of a combination therapy comprising a recombinant adeno-virus associated (rAAV) and an anti-inflammatory steroid for the preparation of a medicament for treating a Limb Girdle Muscular Dystrophy in a human subject in need thereof, wherein the rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05, such that rAAV and the anti-inflammatory steroid of the medicament are co-administered separately, such as the rAAV and anti-inflammatory steroid are co-administered simultaneously or sequentially. The disclosure also provides for a combination therapy for treating Limb Girdle Muscular Dystrophy wherein the combination therapy comprises a recombinant adeno-virus associated (rAAV) and an anti-inflammatory steroid, wherein the rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05, wherein rAAV and the anti-inflammatory steroid are co-administered separately, such as the rAAV and anti-inflammatory steroid are co-administered simultaneously or sequentially. For example, the anti-inflammatory steroid is a glucocorticoid. In some embodiments, the anti-inflammatory steroid is prednisone, prednisolone, betamethasone, dexamethasone, hydrocortisone, methylprednisolone or deflazacort. In some embodiments, the anti-inflammatory steroid is administered orally. The anti-inflammatory steroid may be administered both prior to and after administration of the rAAV. Alternatively, the anti-inflammatory steroid is administered only prior to or only after administration of the rAAV.

For example, in any of the methods, uses or combination therapies disclosed herein, the anti-inflammatory steroid is administered about 12 hour prior to administration of the rAAV or about 24 hours prior to administration of the rAAV or about 36 hours prior to administration of the rAAV or about 48 hours prior to administration of the rAAV or about 60 hours prior to administration of the rAAV or about 72 hours prior to administration of the rAAV or about 96 hours prior to administration. In some embodiments, the inflammatory steroid is administered about 5 days hours prior to administration of the rAAV, about 6 days hours prior to administration of the rAAV, about 7 days hours prior to administration of the rAAV, or about 8 days prior to administration of the rAAV, or about 9 days prior to administration of the rAAV, or about 10 days prior to administration of the rAAV, or about 11 days prior to administration of the rAAV, or about 12 days prior to administration of the rAAV, or about 13 days prior to administration of the rAAV, or about 14 days prior to administration of the rAAV, or about 30 days prior to administration of the rAAV.

In addition, in any of the disclosed methods, uses or combination therapies, the anti-inflammatory steroid is administered at least once a day for about 7 days prior to administration of the rAAV, or administered at least once a day for about 14 days prior to administration of the rAAV, or administered at least once a day for 21 days, or administered at least once a day for about 28 days prior to administration of the rAAV, or administered at least once a day for about 30 days prior to administration of the rAAV, or administered at least once a day for about 45 days prior to administration of the rAAV, or administered at least once a day for about 60 days prior to administration of the rAAV. In some embodiments, the anti-inflammatory steroid is administered 30 to 60 days prior to administration of the rAAV.

For example, in any of the disclosed methods, uses or combination therapies, the anti-inflammatory steroid is administered prior to administration of the rAAV and the anti-inflammatory steroid is administered at least once a day from day 1 to 30 days after administration of the rAAV or at least once a day from 1 to 60 days after administration of the rAAV or at least once a day from 1 to 7 days after administration of the rAAV or at least once a day from 1 to 14 days after administration of the rAAV or at least once a day from 1 to 21 days after administration of the rAAV, or at least once a day from 1 to 24 days after administration of the rAAV, or at least once a day from 1 to 28 days after administration of the rAAV, or at least from 1 to 30 days after administration of the rAAV, or at least 30 to 60 days after administration of the rAAV.

In any of the methods, uses or combination therapies disclosed herein, an anti-CD20 specific antibody is administered prior to administration of the rAAV. In some embodiments, the anti-CD20 specific antibody is administered at least 7 days prior to administration of the rAAV. The term anti-CD20 specific antibody refers to an antibody that specifically binds to or inhibits or reduces the expression or activity of CD20. Exemplary anti-CD20 antibodies include rituximab, ocrelizumab or ofatumumab.

In any of the disclosed methods, uses or combination therapies, an anti-CD20 specific antibody is administered about 60 days prior to administration of the rAAV, or about 45 days prior to administration the rAAV, or about 30 days prior to administration of the rAAV, about 14 days prior to administration of the rAAV, about 7 days prior to administration of the rAAV and within about 24 hours of the administration of the rAAV. In some embodiments, the anti-CD20 antibody is administered 30 to 60 days prior to administration of the rAAV. In some embodiments, the anti-CD20 specific antibody is administered after administration of the rAAV. For example, the anti-CD20 specific antibody is administered both prior to and after administration of the rAAV. Alternatively, the anti-CD20 specific antibody is administered prior to administration of the rAAV or the anti-CD20 specific antibody is administered after administration of the rAAV.

In addition, in any of the disclosed methods, uses or combination therapies may comprise administering an immunosuppressing macrolide. The term immunosuppressing macrolide refers to a macrolide agent suppresses or modulates the immune system of the subject. A macrolide is a classes of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and sirolimus. In some embodiments, the immunosuppressing macrolide is orally administered to the subject. In some embodiments, the immunosuppressing macrolide may be administered both prior to administration of the rAAV and after administration of the rAAV. Alternatively, the immunosuppressing macrolide is administered prior to administration or the rAAV or the immunosuppressing macrolide is administered after administration of the rAAV.

In some embodiments, the immunosuppressing macrolide is administered at least once a day for at least three days prior to administration of the rAAV, or administered at least 4 days prior to administration of the rAAV, or administered at least 5 days prior to administration of the rAAV, or administered at least 6 days prior to administration of the rAAV, administered at least 7 days prior to administration of the rAAV, or administered at least 10 days prior to administration of the rAAV, or administered at least 14 days prior to administration, or administered at least 30 days prior to administration of the rAAV, or administered at least 45 days prior to administration of the rAAV, or administered at least 60 days prior to administration of the rAAV. In some embodiments, the immunosuppressing macrolide is administered 30 to 60 days prior to administration of the rAAV.

The disclosure also provides for a method of treating muscular dystrophy in a human subject in need thereof comprising administering a recombinant adeno-virus associated (rAAV) rAAV.MHCK7.microdystrophin and an immunosuppressing regimen, wherein the immunosuppressing regimen comprises administering one or more of an anti-inflammatory steroid, an anti-CD20 antibody, and an immunosuppressing macrolide. The disclosure also provides for use of a combination therapy comprising a recombinant adeno-virus associated (rAAV) rAAV.MHCK7.microdystrophin and an immunosuppressing regimen for the preparation of a medicament for treating muscular dystrophy in a human subject in need, wherein the immunosuppressing regimen comprises administering one or more of an anti-inflammatory steroid, an anti-CD20 antibody, and an immunosuppressing macrolide, e.g. in the disclosed medicament the rAAV and one or more of the components of the immunosuppressing regimen are co-administered separately, such as the rAAV and one or more components to the immunosuppressing regiment are co-administered simultaneously or sequentially. The disclosure also provides for combination therapy for treating muscular dystrophy in a human subject in need, wherein the combination therapy comprises a recombinant adeno-virus associated (rAAV) rAAV.MHCK7.microdystrophin and an immunosuppressing regimen, wherein the rAAV and the immunosuppressing regimen are co-administered separately, such as the rAAV and one of more of the components of the immunosuppressing regimen co-administered simultaneously or sequentially. The term immunosuppressing regimen refers to a method of treatment or therapy which suppresses or modulates the immune system of the subject. The regimen comprises administration of one or more immune suppressing agents. In some embodiments, the immunosuppressing regimen comprises administering an anti-inflammatory steroid, an anti-CD20 antibody, and an immunosuppressing macrolide.

The disclosure also provides for a method of treating a Limb-Girdle muscular dystrophy in a human subject in need thereof comprising administering a recombinant adeno-virus associated (rAAV) selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; and an immunosuppressing regimen, wherein the immunosuppressing regimen comprises administering one or more of an anti-inflammatory steroid, an anti-CD20 antibody, and an immunosuppressing macrolide. The disclosure also provides for use of a combination therapy comprising a rAAV and an immunosuppressing regimen for the preparation of a medicament for treating a Limb-Girdle muscular dystrophy in a human subject in need, wherein the rAAV comprises a rAAV recombinant adeno-virus associated (rAAV) is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; and wherein the immunosuppressing regimen comprises administering one or more of an anti-inflammatory steroid, an anti-CD20 antibody, and an immunosuppressing macrolide, e.g. in the disclosed medicament the rAAV and one or more components of the immunosuppressing regimen are co-administered separately, such as the rAAV and one or more of components of the immunosuppressing regimen are co-administered simultaneously or sequentially. The disclosure also provides for a combination therapy for treating a Limb-Girdle muscular dystrophy in a human subject in need thereof, wherein the combination therapy comprises a recombinant adeno-virus associated (rAAV) selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; and wherein the immunosuppressing regimen comprises administering one or more of an anti-inflammatory steroid, an anti-CD20 antibody, and an immunosuppressing macrolide wherein the rAAV and one or more of the components of the immunosuppressing regimen are co-administered separately, such as the rAAV and one or more of the components of the immunosuppressing regimen are co-administered simultaneously or sequentially. In some embodiments, the immunosuppressing regimen comprises administering an anti-inflammatory steroid, an anti-CD20 antibody, and an immunosuppressing macrolide.

In one exemplary immunosuppressing regimen, an anti-inflammatory steroid is administered about 24 hours prior to administration of the rAAV. In another exemplary immunosuppressing regimen, an anti-inflammatory steroid is administered prior to administration of the rAAV and the anti-inflammatory steroid is administered at least once a day from day 1 to 30 days after administration of the rAAV or the anti-inflammatory steroid is administered at least once a day from day 1 to 60 days after administration of the rAAV.

In any of the disclosed immunosuppressing regimens, the anti-inflammatory steroid is a glucocorticoid such as prednisone, prednisolone, betamethasone, dexamethasone, hydrocortisone, methylprednisolone or deflazacort. In some embodiments, the anti-inflammatory steroid is administered orally.

In additional exemplary immunosuppressing regimens, an anti-CD20 specific antibody prior to administration of the rAAV. For example, the anti-CD20 antibody is administered by intravascular infusion. Exemplary anti-CD20 specific antibody include rituximab, ocrelizumab or ofatumumab.

In one embodiment, the anti-CD20 specific antibody is administered at least 14 days prior to administration of the rAAV. In another embodiment, the anti-CD20 specific antibody is administered about 60 days prior to administration of the rAAV, about 45 days prior to administration of the rAAV, about 30 days prior to administration of the rAAV, 14 days prior to administration of the rAAV, about 7 days prior to administration of the rAAV and within about 24 hours of the administration of the rAAV. In addition, the anti-CD20 specific antibody administered for 30 to 60 days prior to administration of the rAAV. The disclosed immunosuppressing regimens also include administering an anti-CD20 specific antibody after administration of the rAAV.

In addition, the disclosed immunosuppressing regimens comprise administering an immunosuppressing macrolide at least once a day for at least three days prior to administration of the rAAV. The immunosuppressing regimens also may comprise administering an immunosuppressing macrolide after administration of the rAAV. In any of the disclosed immunosuppressing regimen, the immunosuppressing macrolide is administered orally. Exemplary immunosuppressing macrolides include tacrolimus, pinecrolimus or sirolimus.

In some embodiments, the disclosed immunosuppressing regimen is administered from 30 to 60 days prior to administration of the rAAV. In addition, the immunosuppressing regimen is administered about 60 days prior to administration of the rAAV, about 45 days prior to administration of the rAAV, about 30 days prior to administration the rAAV, about 14 days prior to administration of the rAAV, about 7 days prior to administration of the rAAV, about 24 hours prior to administration of the rAAV.

In a particular embodiment, the disclosure provides for methods of treating muscular dystrophy in a human subject in need thereof comprising administering a recombinant adeno-virus associated (rAAV) rAAV.MHCK7.microdystrophin and an immunosuppressing regimen, wherein the immunosuppressing regimen comprises the steps of i) orally administering an anti-inflammatory steroid about 24 hours prior to administration of the rAAV, and administering an anti-inflammatory steroid at least once a day from day 1 to 30 days after administration of the rAAV or administering an the anti-inflammatory steroid at least once a day from day 1 to 60 days after administration of the rAAV, ii) intravenously administering an anti-CD20 antibody about 14 days prior to administration of the rAAV, about 7 days prior to administration of the rAAV and within about 24 hours of the administration of the rAAV, and optionally administering the anti-CD20 antibody after administration of the rAAV, iii) orally administering an immunosuppressing macrolide at least once a day for at least three days prior to administration of the rAAV, and optionally administering the an immunosuppressing macrolide after administration of the rAAV. For example, the anti-inflammatory steroid is prednisone, prednisolone, betamethasone, dexamethasone, hydrocortisone, methylprednisolone or deflazacort, the anti-CD20 specific antibody is rituximab, ocrelizumab or ofatumumabone or more of an anti-inflammatory steroid, an anti-CD20 antibody, and an immunosuppressing macrolide, the immunosuppressing macrolide is tacrolimus, pinecrolimus or sirolimus. In an exemplary embodiment, the immunosuppressing regimen comprises the anti-inflammatory steroid prednisone or prednisolone, the anti-CD20 antibody rituximab, and the immunosuppressing macrolide sirolimus.

The also provides for use of a combination therapy comprising a rAAV and an immunosuppressing regimen for the preparation of a medicament for the treating muscular dystrophy in a human subject in need thereof, wherein the rAAV is a rAAV.MHCK7.microdystrophin and the immunosuppressing regimen comprises i) orally administering an anti-inflammatory steroid about 24 hours prior to administration of the rAAV, and administering an anti-inflammatory steroid at least once a day from day 1 to 30 days after administration of the rAAV or administering an the anti-inflammatory steroid at least once a day from day 1 to 60 days after administration of the rAAV, ii) intravenously administering an anti-CD20 antibody about 14 days prior to administration of the rAAV, about 7 days prior to administration of the rAAV and within about 24 hours of the administration of the rAAV, and optionally administering the anti-CD20 antibody after administration of the rAAV, iii) orally administering an immunosuppressing macrolide at least once a day for at least three days prior to administration of the rAAV, and optionally administering the an immunosuppressing macrolide after administration of the rAAV. For example, the anti-inflammatory steroid is prednisone, prednisolone, betamethasone, dexamethasone, hydrocortisone, methylprednisolone or deflazacort, the anti-CD20 specific antibody is rituximab, ocrelizumab or ofatumumabone or more of an anti-inflammatory steroid, an anti-CD20 antibody, and an immunosuppressing macrolide, the immunosuppressing macrolide is tacrolimus, pinecrolimus or sirolimus. In an exemplary embodiment, the immunosuppressing regimen comprises the anti-inflammatory steroid prednisone or prednisolone, the anti-CD20 antibody rituximab, and the immunosuppressing macrolide sirolimus.

The disclosure provides for a combination therapy comprising a rAAV and an immunosuppressing regimen for treating muscular dystrophy in a human subject in need thereof, wherein the rAAV is a rAAV.MHCK7.microdystrophin and the immunosuppressing regimen, comprises i) orally administering an anti-inflammatory steroid about 24 hours prior to administration of the rAAV, and administering an anti-inflammatory steroid at least once a day from day 1 to 30 days after administration of the rAAV or administering an the anti-inflammatory steroid at least once a day from day 1 to 60 days after administration of the rAAV, ii) intravenously administering an anti-CD20 antibody about 14 days prior to administration of the rAAV, about 7 days prior to administration of the rAAV and within about 24 hours of the administration of the rAAV, and optionally administering the anti-CD20 antibody after administration of the rAAV, iii) orally administering an immunosuppressing macrolide at least once a day for at least three days prior to administration of the rAAV, and optionally administering the an immunosuppressing macrolide after administration of the rAAV. For example, the anti-inflammatory steroid is prednisone, prednisolone, betamethasone, dexamethasone, hydrocortisone, methylprednisolone or deflazacort, the anti-CD20 specific antibody is rituximab, ocrelizumab or ofatumumabone or more of an anti-inflammatory steroid, an anti-CD20 antibody, and an immunosuppressing macrolide, the immunosuppressing macrolide is tacrolimus, pinecrolimus or sirolimus. In an exemplary embodiment, the immunosuppressing regimen comprises the anti-inflammatory steroid prednisone or prednisolone, the anti-CD20 antibody rituximab, and the immunosuppressing macrolide sirolimus.

In another particular embodiment, the disclosure provides for a method of treating a Limb-Girdle muscular dystrophy in a human subject in need thereof comprising administering a recombinant adeno-virus associated (rAAV) selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; and an immunosuppressing regimen, wherein the immunosuppressing regimen comprises the steps of i) orally administering an anti-inflammatory steroid about 24 hours prior to administration of the rAAV, and administering an anti-inflammatory steroid at least once a day from day 1 to 30 days after administration of the rAAV or the anti-inflammatory steroid is administered at least once a day from day 1 to 60 days after administration of the rAAV, ii) intravenously administering an anti-CD20 antibody about 14 days prior to administration of the rAAV, about 7 days prior to administration of the rAAV and within about 24 hours of the administration of the rAAV, and optionally administering the anti-CD20 antibody after administration of the rAAV, iii) orally administering an immunosuppressing macrolide at least once a day for at least three days prior to administration of the rAAV, and optionally administering the an immunosuppressing macrolide after administration of the rAAV.

In another particular embodiment, the disclosure provides for a use of a combination therapy comprising a rAAV and an immunosuppressing regimen for the preparation of a medicament for treating a Limb-Girdle muscular dystrophy in a human subject in need thereof, wherein the rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; and the immunosuppressing regimen comprises i) orally administering an anti-inflammatory steroid about 24 hours prior to administration of the rAAV, and administering an anti-inflammatory steroid at least once a day from day 1 to 30 days after administration of the rAAV or the anti-inflammatory steroid is administered at least once a day from day 1 to 60 days after administration of the rAAV, ii) intravenously administering an anti-CD20 antibody about 14 days prior to administration of the rAAV, about 7 days prior to administration of the rAAV and within about 24 hours of the administration of the rAAV, and optionally administering the anti-CD20 antibody after administration of the rAAV, iii) orally administering an immunosuppressing macrolide at least once a day for at least three days prior to administration of the rAAV, and optionally administering the an immunosuppressing macrolide after administration of the rAAV.

In another particular embodiment, the disclosure provides for a combination therapy comprising a rAAV and an immunosuppressing regimen for treating a Limb-Girdle muscular dystrophy in a human subject in need thereof, wherein the rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; and the immunosuppressing regimen comprises i) orally administering an anti-inflammatory steroid about 24 hours prior to administration of the rAAV, and administering an anti-inflammatory steroid at least once a day from day 1 to 30 days after administration of the rAAV or the anti-inflammatory steroid is administered at least once a day from day 1 to 60 days after administration of the rAAV, ii) intravenously administering an anti-CD20 antibody about 14 days prior to administration of the rAAV, about 7 days prior to administration of the rAAV and within about 24 hours of the administration of the rAAV, and optionally administering the anti-CD20 antibody after administration of the rAAV, iii) orally administering an immunosuppressing macrolide at least once a day for at least three days prior to administration of the rAAV, and optionally administering the an immunosuppressing macrolide after administration of the rAAV.

In another embodiment, the disclosure provides methods of treating muscular dystrophy in a human subject in need thereof comprising subjecting the subject's plasma to at least one therapeutic plasma exchange (TPE) prior to administration of a second dose of recombinant adeno-virus associated (rAAV) rAAV.MHCK7.microdystrophin, wherein the subject was administered a first dose of rAAV prior to being subjected to TPE.

In another embodiment, the disclosure provides for use of a combination therapy for treating muscular dystrophy in a human subject in need thereof, wherein the combination therapy comprises subjecting the subject's plasma to at least one therapeutic plasma exchange (TPE) prior to administration of a second dose of recombinant adeno-virus associated (rAAV) rAAV.MHCK7.microdystrophin, wherein the subject was administered a first dose of rAAV prior to being subjected to TPE.

In another embodiment, the disclosure provides for a combination therapy for treating muscular dystrophy in a human subject in need thereof, wherein the combination therapy comprises subjecting the subject's plasma to at least one therapeutic plasma exchange (TPE) prior to administration of a second dose of recombinant adeno-virus associated (rAAV) rAAV.MHCK7.microdystrophin, wherein the subject was administered a first dose of rAAV prior to being subjected to TPE.

The disclosure provides for a method of treating a Limb-Girdle muscular dystrophy in a human subject in need thereof comprising subjecting the subject's plasma to at least one therapeutic plasma exchange (TPE) prior to administration of a second dose of recombinant adeno-virus associated (rAAV) selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; wherein the subject was administered a first dose of rAAV prior to being subjected to TPE.

In a further embodiment, the disclosure provides methods of treating muscular dystrophy in a human subject in need thereof comprising the steps of a) administering a first dose of recombinant adeno-virus associated (rAAV) rAAV.MHCK7.microdystrophin, b) subjecting the subject's plasma to at least one therapeutic plasma exchange (TPE), and c) administering a second dose of rAAV. In any of the disclosed methods, the subject's plasmas is subject to at least two TPE or at least three TPE prior to administration of the 2^{nd} dose or rAAV. In some embodiments, the subject's plasma is subject to at least four TPE prior to administration of the 2^{nd} dose of rAAV, or the subject's plasma is subject five TPE prior to administration of the 2^{nd} dose of rAAV, or the subject's plasma is subject six TPE prior to administration of the 2^{nd} dose of rAAV, or the subject's plasma is subject seven TPE prior to administration of the 2^{nd} dose of rAAV.

In another embodiment, the disclosure provides for a method of treating a Limb Girdle muscular dystrophy in a human subject in need thereof comprising the steps of a) administering a first dose of recombinant adeno-virus associated selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; b) subjecting the subject's plasma to at least one therapeutic plasma exchange (TPE), and c) administering a second dose or rAAV. In any of the disclosed methods, the subject's plasmas is subject to at least two TPE or at least three TPE prior to administration of the 2^{nd} dose or rAAV. In some embodiments, the subject's plasma is subject to at least four TPE prior to administration of the 2^{nd} dose of rAAV, or the subject's plasma is subject five TPE prior to administration of the 2^{nd} dose of rAAV, or the subject's plasma is subject six TPE prior to administration of the 2^{nd} dose of rAAV, or the subject's plasma is subject seven TPE prior to administration of the 2^{nd} dose of rAAV.

In a further embodiment, the disclosure provides for methods of treating muscular dystrophy in a human subject in need thereof comprising the steps of a) subjecting the subject's plasma to at least one therapeutic plasma exchange (TPE) prior to administering recombinant adeno-virus associated (rAAV) rAAV.MHCK7.microdystrophin, and b) administering rAAV. In any of the disclosed methods, the subject's plasma is subjected to at least two TPE prior to administering the rAAV, at least three TPE prior to administering the rAAV, at least four TPE prior to administering the rAAV, at least five TPE prior to administering the rAAV, at least six TPE prior to administering the rAAV or at least seven TPE prior to administering prior to administering the rAAV. In these disclosed methods, the subject is administered an anti-inflammatory steroid about 24 hours prior to administration of the rAAV. In addition, in some embodiments, the subject is administered an anti-inflammatory steroid at least once a day from day 1 to 60 days after administration of the rAAV. For example, the anti-inflammatory steroid is administered orally. In addition, the anti-inflammatory steroid is a glucocorticoid such as prednisone, prednisolone, betamethasone, dexamethasone, hydrocortisone, methylprednisolone or deflazacort.

In addition, the disclosure provides for methods of treating Limb Girdle muscular dystrophy in a human subject in need thereof comprising the steps of muscular dystrophy in a human subject in need thereof comprising the steps of a) subjecting the subject's plasma to at least one therapeutic plasma exchange (TPE) prior to administering recombinant adeno-virus associated (rAAV) selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; b administering rAAV. In any of the disclosed methods, the subject's plasma is subjected to at least two TPE prior to administering the rAAV, at least three TPE prior to administering the rAAV, at least four TPE prior to administering the rAAV, at least five TPE prior to administering the rAAV, at least six TPE prior to administering the rAAV or at least seven TPE prior to administering prior to administering the rAAV. In these disclosed methods, the subject is administered an anti-inflammatory steroid about 24 hours prior to administration of the rAAV. In addition, in some embodiments, the subject is administered an anti-inflammatory steroid at least once a day from day 1 to 60 days after administration of the rAAV. For example, the anti-inflammatory steroid is administered orally. In addition, the anti-inflammatory steroid is a glucocorticoid such as prednisone, prednisolone, betamethasone, dexamethasone, hydrocortisone, methylprednisolone or deflazacort.

In any of the disclosed methods, the subject's plasma is subjected to TPE for at least 9 days prior to administration of the rAAV, at least 7 days prior to administration, 5 days prior to administration, or 2 days prior to administration. In addition, there is about 24 to about 48 hours between sessions of TPE carried out on the subject's plasma prior to administration of the rAAV. In a particular embodiment, the subject's plasma is subjected to at least two TPE prior to administration of the rAAV, wherein there is about 48 hours between the TPE.

In any of the method described herein, the subject has a level of anti-AAVrh.74 antibodies of about 1:400 or less at the time of administration of the rAAV. For example, the subject has a level of anti-AAVrh.74 antibodies of about 1:100 to about 1:400 at the time of administration of the rAAV or a level of anti-AAVrh.74 antibodies of about 1:100 to 1:300, or a level of anti-AAVrh.74 antibodies of about 1:100 to 1:200, or a level of anti-AAVrh.74 antibodies of about 1:250 to 1:500, or a level of anti-AAVrh.74 antibodies of about 1:200 to 1:400. The antibody titer is determined as total antibody binding titer. In any of the disclosed methods of treating muscular dystrophy, these methods further comprise a step of determining the presence of anti-AAVrh.74 antibodies in serum or plasma of said subject. The step of determining the present of anti-AAVrh.74 antibodies may be carried out before administration of rAAV, after administration of rAAV, before an immune response or adverse event is observed or after an immune response or adverse event is observed. In addition, the determining step may be performed prior to the step of administering an immunosuppressing regimen or TPE. For example, the determining step is performed prior to any administration of any AAV to said subject or the determining step is performed prior to administration of any AAVrh.74 to said subject.

The disclosure also provides for methods further comprising a step of comparing the level of anti-AAVrh.74 antibodies in serum or plasma of said subject to a positive control. For example, the positive control utilizes an anti-AAVrh.74 monoclonal antibody.

In any of the disclosed methods, the determination of the presence of anti-AAVrh.74 may be determined utilizing an immunofluorescence assay, an immunohistochemical assay, a Western blot, a direct enzyme-linked immunosorbent assay (ELISA), an indirect ELISA, a sandwich ELISA, a competitive ELISA, a reverse ELISA, a chemiluminescence assay, a radioimmunoassay, or an immunoprecipitation assay.

In any of the disclosed methods, the step of determining the presence of anti-AAVrh.74 antibodies comprises utilizing a monoclonal antibody comprising a VH CDR1 amino acid sequence selected from the group consisting of NYGMN (SEQ ID NO: 20), DYGMN (SEQ ID NO: 22), YTFTNYGMN (SEQ ID NO: 21), and YTFTKYGMN (SEQ ID NO: 23), or a monoclonal antibody comprising a VH CDR2 amino acid sequence selected from the group consisting of WINTYTGEPTYADDFKG (SEQ ID NO: 24), WINTNTGEPTYGDDFKG (SEQ ID NO:25), and WMGWINTYTGEPTY (SEQ ID NO: 26), or a monoclonal antibody comprising a VH CDR3 amino acid sequence selected from the group consisting of GVAHYSDSRFAFDY (SEQ ID NO: 27), GNAHPGGSAFVY (SEQ ID NO: 28), RGSYYYDSSPAWFAY (SEQ ID NO: 29), RGVDSSGYGAFAY (SEQ ID NO: 30), and TRGTSTMISTFAFVY (SEQ ID NO: 31), or a monoclonal antibody comprising VL CDR1 amino acid sequence selected from the group consisting of SVSSSVSYMH (SEQ ID NO: 32), SASSGVTYMH (SEQ ID NO: 33), SSVSYMH (SEQ ID NO: 34), and SSVRYMH (SEQ ID NO: 35), or a monoclonal antibody comprising a VL CDR2 amino acid sequence selected from the group consisting of YTSNLAS (SEQ ID NO: 36), RTSNLAS (SEQ ID NO: 37), LWIYSTSNLAS (SEQ ID NO: 38), and VWIYSTSNLAS (SEQ ID NO: 39), or a monoclonal antibody comprising a VH CDR3 amino acid sequence selected from the group consisting of QQRSSYPFT (SEQ ID NO: 40), QQRSTYPF (SEQ ID NO: 41), QQRSFYPF (SEQ ID NO: 42), and QQRTYYPF (SEQ ID NO: 43).

In an exemplary embodiment, the disclosed methods comprise a step of determining the presence of anti-AAVrh.74 antibodies utilizing an anti-AAVrh.74 monoclonal antibody such as a monoclonal antibody comprising a variable heavy chain (VH) sequence set forth in SEQ ID NO: 10, 12, 14, 16, or 18, or a monoclonal antibody comprising a variable light chain (VL) sequence set forth in SEQ ID NO: 11, 13, 15, 17, or 19.

In a further embodiment, the disclosed methods comprise a step of determining the presence of anti-AAVrh.74 antibodies utilizing an anti-AAVrh.74 monoclonal antibody comprising a variable heavy chain (VH) sequence set forth in SEQ ID NO: 10, 12, 14, 16, or 18, and a variable light chain (VL) sequence set forth in SEQ ID NO: 11, 13, 15, 17, or 19.

In any of the disclosed methods, the step of determining the presence of anti-AAVrh.74 antibodies is quantitative, wherein said subject is identified as seropositive for anti-AAVrh.74 antibodies based said quantitation, and wherein said immunosuppressing regimen or TPE is selectively administered to the seropositive subject. In any of the methods disclosed herein, the rAAV is administered by a systemic route of administration at a dose of about 5.0×10¹² vg/kg to about 1.0×10¹⁵ vg/kg. The muscular dystrophy may be Duchenne muscular dystrophy or Becker's muscular dystrophy.

For example, the dose of rAAV administered is about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg.

In one embodiment, the methods of the disclosure comprise systemically administering rAAV wherein the systemic route of administration is an intravenous route and the dose of the rAAV administered is about 2.0×10¹⁴ vg/kg. In another embodiment, the methods of the disclosure comprise systemically administering rAAV wherein the systemic route of administration is an intravenous route and the dose of the rAAV administered is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵ vg/kg. In one embodiment, the rAAV is AAVrh74.MHCK7.microdystrophin or AAVrh74.MCK.microdystrophin. In one embodiment, the rAAV is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one embodiment, the rAAV is the AAVrh74.MCK.microdystrophin of nucleotides 56-4820 of SEQ ID NO: 5.

In any of the methods, combination therapies or uses of the disclosure, the dose of rAAV can be administered at about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/k, or about 10 mL/kg to 15 mL/kg or 10 mL/kg to about 20 mL/kg. In a particular embodiment, the dose or the rAAV is administered in about 10 mL/kg. In one embodiment, the rAAV is AAVrh74.MHCK7.microdystrophin or AAVrh74.MCK.microdystrophin. In one embodiment, the rAAV is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one embodiment, the rAAV is the AAVrh74.MCK.microdystrophin of nucleotides 56-4820 of SEQ ID NO: 5.

In any of the methods, combination therapies or medicaments of the disclosure, the dose of rAAV can be administered by injection, infusion or implantation. For example, the dose of rAAV is administered by infusion over approximately one hour. In addition, the dose of rAAV is administered by an intravenous route through a peripheral limb vein, such as a peripheral arm vein or a peripheral leg vein. Alternatively, the infusion may be administered over approximately 30 minutes, or approximately 1.5 hours, or approximately 2 hours, or approximately 2.5 hours or approximately 3 hours. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

The rAAV administered by any of the methods, combination therapies or uses of the disclosure can comprise the human micro-dystrophin nucleotide sequence of SEQ ID NO: 1, the MHCK7 promoter sequence of SEQ ID NO: 2 or SEQ ID NO: 7. In addition, the rAAV administered by any of the methods of the disclosure comprises the human micro-dystrophin nucleotide sequence of SEQ ID NO: 1 and the MHCK7 promoter sequence of SEQ ID NO: 2 or SEQ ID NO: 7. For example, the rAAV can comprise the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6.

In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the methods, combination therapies or uses of the disclosure, the rAAV administered is of the serotype for AAVrh.74 antibodies.

In some embodiments, the methods, combination therapies or use of the disclosure treat Duchenne muscular dystrophy or Becker's muscular dystrophy. An exemplary embodiment is a method, combination therapy or medicament for treating Duchenne muscular dystrophy or Becker's muscular dystrophy in a human subject in need thereof comprising the step of administering a dose recombinant adeno-virus associated (rAAV) rAAV.MHCK7.microdystrophin, wherein the route of administration is intravenous infusion and the dose of the rAAV administered is about 2×10¹⁴ vg/kg over approximately one hour, and wherein the rAAV vector comprises the AAVrh74.MHCK7.micro-dystrophin construct nucleotide sequence of SEQ ID NO: 9 or of nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9 or of nucleotides 55-5021 of SEQ ID NO: 3, nucleotides 1-4977 of SEQ ID NO: 8, or nucleotides 56-5022 of SEQ ID NO: 6. in one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In one embodiment, the disclosure provides for a rAAV comprising a muscle specific control element nucleotide sequence, and a nucleotide sequence encoding the micro-dystrophin protein. For example, the nucleotide sequence encodes a functional micro-dystrophin protein, wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 1, wherein the protein retains micro-dystrophin activity. The micro-dystrophin protein provides stability to the muscle membrane during muscle contraction, e.g. micro-dystrophin acts as a shock absorber during muscle contraction. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9 or of nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

The disclosure also provides for rAAV wherein the nucleotide sequence comprises a nucleotide sequence that hybridizes under stringent conditions to the nucleic acid sequence of SEQ ID NO: 1, or compliments thereof, and encodes a functional micro-dystrophin protein.

In one embodiment, the rAAV is a non-replicating, recombinant adeno-associated virus (AAV) termed AAVrh74.MHCK7.micro-dystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. This vector genome contains minimal elements required for gene expression, including AAV2 inverted terminal repeats (ITR), the micro-dystrophin, SV40 intron (SD/SA), and synthetic polyadenylation (Poly A) signal, all under the control of the MHCK7 promoter/enhancer. The schematic of the genome and expression cassette is shown FIG. 1. The AAVrh.74 serotype can be employed to achieve efficient gene transfer in skeletal and cardiac muscle following IV administration.

In another embodiment, the disclosure provides for a method of treating a Limb Girdle muscular dystrophy in a human subject in need thereof comprising administering a rAAV comprising the nucleotide sequence of SEQ ID NO: 44.

In another aspect, this disclosure provides a method, combination therapy or use for treating a limb-girdle muscular dystrophy in a subject in need, comprising administering to the subject an rAAV intravenous infusion over approximately 1 to 2 hours at a dose of about 5.0×10¹³ vg/kg or about 2.0×10¹⁴ vg/kg based on a supercoiled plasmid as the quantitation standard, or about 1.85×10¹³ vg/kg or 7.41×10¹³ vg/kg based on a linearized plasmid as the quantitation standard, and wherein the rAAV comprises a nucleotide sequence of SEQ ID NO: 44. In another aspect, the disclosure describes a method of expressing beta-sarcoglycan gene in a subject's cell comprising administering to the subject the scAAVrh74.MHCK7.hSGCB construct that comprises a nucleotide sequence that is at least 90%, 95%, or 99% identical to SEQ ID NO: 19. In one aspect, the disclosure provides a method of increasing beta-sarcoglycan positive fibers and/or decreasing CK level in a subject's muscle tissue comprising administering to the subject the scAAVrh74.MHCK7.hSGCB construct nucleotide sequence that is at least 90%, 95%, or 99% identical to SEQ ID NO: 44.

In another aspect, described here in a recombinant AAV vector comprising a polynucleotide sequence encoding β-sarcoglycan. In some embodiments, the polynucleotide sequence encoding β-sarcoglycan comprises a sequence e.g. at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence set forth in SEQ ID NO: 45 and encodes protein that retains β-sarcoglycan activity. In some embodiments, the polynucleotide sequence encoding β-sarcoglycan comprises the nucleotide sequence set forth in SEQ ID NO: 45. In some embodiments, the polynucleotide sequence encoding β-sarcoglycan consists of the nucleotide sequence set forth in SEQ ID NO: 45.

In another aspect, a recombinant AAV vector described herein comprises a polynucleotide sequence encoding β-sarcoglycan that is at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to the amino acid sequence of SEQ ID NO: 46, and the protein retains β-sarcoglycan activity.

In another aspect, a recombinant AAV vector described herein comprises a polynucleotide sequence encoding β-sarcoglycan that has the amino acid sequence of SEQ ID NO: 46.

In another aspect, described herein is a recombinant AAV vector comprising a polynucleotide sequence encoding functional β-sarcoglycan that comprises a nucleotide sequence that hybridizes under stringent conditions to the nucleic acid sequence of SEQ ID NO: 45, or a complement thereof.

In a particular embodiment, the disclosure provides for methods, combination therapies or uses for treating LGMD2E in a human subject in need thereof comprising administering the rAAV scAAVrh74.MHCK7.HSGCB. For example, in any of the methods of treating LGMD2E, scAAVrh74.MHCK7.HSGCB is administered by intravenous infusion at a dose of about 0.5×10¹⁴ vg/kg or about 2×10¹⁴ vg/kg. The dosages stated herein in reference to scAAVrh74.MHCK7.HSGCB including in the immediate forgoing to are based on utilizing supercoiled qPCR standards. Respectively, 0.5×10¹⁴ vg/kg and 2×10¹⁴ vg/kg correspond to 1.85×10¹³ and 7.41×10¹³ vg/kg as measured by utilizing linear qPCR standards.

In a particular embodiment, in any of the methods, combination therapies or uses for treating a Limb Girdle muscular dystrophy in a human subject, the human subject is suffering from LGMD2E, and the rAAV is administered by intravenous infusion at a dose of about 2×10¹⁴ vg/kg, and wherein the rAAV comprises the rAAV is scAAVrh74.MHCK7.HSGCB comprising the nucleotide sequence of SEQ ID NO: 44.

In a related embodiment, the human subject is suffering from the muscular dystrophy LGMD2E and the rAAV is scAAVrh74.MHCK7.HSGCB administered by intravenous infusion at a dose of about 2×10¹⁴ vg/kg, the method further comprising administering to the subject 1 mg/kg/day of prednisone beginning one day (12 to 24 hours) prior to the administration of the rAAV and continuing with or without tapering for sixty day.

In another related embodiment, the human subject is suffering from the muscular dystrophy LGMD2E and the rAAV is scAAVrh74.MHCK7.HSGCB administered by intravenous infusion at a dose of about 0.5×10¹⁴ vg/kg, the method further comprising administering to the subject 1 mg/kg/day of prednisone beginning one day (12 to 24 hours) prior to the administration of the rAAV and continuing with or without tapering for thirty days.

It is recognized that the thirty day and the sixty day administrations of prednisone can be tapered, by those skilled in the art according to the particular clinical exhibits of the subject and as otherwise further described herein the methods of the invention.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO4, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

The term “muscle specific control element” refers to a nucleotide sequence that regulates expression of a coding sequence that is specific for expression in muscle tissue. These control elements include enhancers and promoters. The disclosure provides for constructs comprising the muscle specific control elements MCKH7 promoter, the MCK promoter and the MCK enhancer.

In one aspect, the disclosure provides for a rAAV wherein the muscle specific control element is a human skeletal actin gene element, cardiac actin gene element, myocyte-specific enhancer binding factor (MEF), muscle creatine kinase (MCK), truncated MCK (tMCK), myosin heavy chain (MHC), hybrid α-myosin heavy chain enhancer-/MCK enhancer-promoter (MHCK7), C5-12, murine creatine kinase enhancer element, skeletal fast-twitch troponin c gene element, slow-twitch cardiac troponin c gene element, the slow-twitch troponin i gene element, hypoxia-inducible nuclear factors, steroid-inducible element or glucocorticoid response element (GRE).

For example, the muscle specific control element is the MHCK7 promoter nucleotide sequence SEQ ID NO: 2 or SEQ ID NO: 7, or the muscle specific control element is MCK nucleotide sequence SEQ ID NO: 4. In addition, in any of the rAAV vectors of the disclosure, the muscle specific control element nucleotide sequence, e.g. MHCK7 or MCK nucleotide sequence, is operably linked to the nucleotide sequence encoding the micro-dystrophin protein. For example, the MHCK7 promoter nucleotide sequence (SEQ ID NO: 2 or SEQ ID NO: 7) is operably linked to the human micro-dystrophin coding sequence (SEQ ID NO: 1) as set out in the construct provided in FIG. 1 or FIG. 2 (SEQ ID NO: 3) or FIG. 13 (SEQ ID NO: 9). In another example, the MCK promoter (SEQ ID NO: 4) is operably linked to the human micro-dystrophin coding sequence (SEQ ID NO: 1) as set out in the construct provided in FIG. 5 or FIG. 6 (SEQ ID NO: 5). In another aspect, the disclosure provides for a rAAV vector comprising the nucleotide sequence of SEQ ID NO: 1 and SEQ ID NO: 2, or SEQ ID NO: 1 and SEQ ID NO: 7. The disclosure also provides for a rAAV vector comprising the nucleotide sequence of SEQ ID NO: 1 and SEQ ID NO: 4.

In a further aspect, the disclosure provides for an rAAV construct contained in the plasmid comprising the nucleotide sequence of SEQ ID NO: 3, SEQ ID NO: 5, SEQ ID NO: 6, or SEQ ID NO: 8. For example, the AAVrh74.MHCK7.microdystrophin vector comprises the nucleotide sequence within and inclusive of the ITR's of SEQ ID NO: 3 and shown in FIG. 2. The rAAV vector comprises the 5′ ITR, MHCK7 promoter, a chimeric intron sequence, the coding sequence for the human micro-dystrophin gene, polyA, and 3′ ITR. In one embodiment, the vector comprises nucleotides 55-5021 of SEQ ID NO: 3. The plasmid set forth in SEQ ID NO: 3 further comprises ampicillin resistance and the pGEX plasmid backbone with pBR322 origin of replication.

In another aspect, the disclosure provides for a rAAV comprising the nucleotide sequence of SEQ ID NO: 9. for example, the AAVrh74.MHCK7.microdystrophin vector construct comprises the nucleotide sequence of SEQ ID NO: 9 and shown in FIG. 7. This rAAV vector construct comprises the MHCK7 promoter, a chimeric intron sequence, the coding sequence for the human micro-dystrophin gene, and polyA. In one embodiment, the rAAV vector construct further comprises an ITR 5′ to the promoter, and an ITR 3′ to the polyA. In one embodiment, the rAAV is AAVrh.74.

In another aspect, the AAVrh74.MHCK7.microdystrophin vector comprises the nucleotide sequence within and inclusive of the ITR's of SEQ ID NO: 8 and shown in FIG. 9. The rAAV vector comprises the 5′ ITR, MHCK7 promoter, a chimeric intron sequence, the coding sequence for the human micro-dystrophin gene, polyA, and 3′ ITR. In one embodiment, the vector comprises nucleotides 1-4977 of SEQ ID NO: 9. The plasmid set forth in SEQ ID NO: 3 further comprises kanamycin resistance and the pGEX plasmid backbone with pBR322 origin of replication.

In another aspect, the disclosure provides for a plasmid comprising the AAVrh74.MHCK7.microdystrophin vector construct. In one embodiment, the plasmid comprises the 5′ ITR, MHCK7 promoter, a chimeric intron sequence, the coding sequence for the human micro-dystrophin gene, polyA, and 3′ ITR. In one embodiment, the plasmid comprises kanamycin resistance and optionally comprises the pGEX plasmid backbone with pBR322 origin of replication. In a particular embodiment, the plasmid is set forth in SEQ ID NO: 8, and shown in FIGS. 8 and 9.

The disclosure provides for a recombinant AAV vector comprising the human micro-dystrophin nucleotide sequence of SEQ ID NO: 1 and the MHCK7 promoter nucleotide sequence of SEQ ID NO: 2 or SEQ ID NO: 7. This rAAV vector is the AAV serotype AAVrh.74.

The disclosure also provides for a rAAV comprising the AAVrh74.MHCK7.micro-dystrophin construct nucleotide sequence within and inclusive of the ITR's in SEQ ID NO: 3, the nucleotide sequence within and inclusive of the ITR's in SEQ ID NO: 8 or the nucleotide sequence as set forth in SEQ ID NO: 9. This rAAV vector is the AAV serotype AAVrh.74.

The rAAV vectors of the disclosure may be any AAV serotype, such as the serotype AAVrh.74, AAV1, AAV2, AAV4, AAV5, AAV6, AAV7, AAV8, AAV9, AAV10, AAV11, AAV12 or AAV13.

The disclosure also provides for pharmaceutical compositions (or sometimes referred to herein as simply “compositions”) comprising any of the rAAV vectors of the disclosure.

In another embodiment, the disclosure provides for methods of producing a rAAV vector particle comprising culturing a cell that has been transfected with any rAAV vector of the disclosure and recovering rAAV particles from the supernatant of the transfected cells. The disclosure also provides for viral particles comprising any of the recombinant AAV vectors of the disclosure.

In any of the methods of treating a muscular dystrophy, the level of the transgene of interest, such as beta-sarcoglycan gene expression or micro-dystrophin gene expression, in a cell of the subject is increased after administration of the rAAV. Expression of the transgene of interest gene in the cell is detected by measuring the protein of interest level by Western blot in muscle biopsied before and after administration of the rAAV. In particular, the level of the protein of interest is increased by at least about 70% to at least about 80%, or at least about 70% to at least about 90%, or at least about 80% to at least about 90% after administration of rAAV compared to the level of the protein of interest before administration of rAAV. For example, the level of the protein of interest is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of rAAV compared to the level of the protein of interest before administration of rAAV.

In addition, expression of the micro-dystrophin gene in the cell is detected by measuring the level of the protein of interest by immunohistochemistry in muscle biopsies before and after administration of the rAAV. The level of the protein of interest is increased by at least about 70% to at least about 80%, or at least about 70% to at least about 90%, or at least about 80% to at least about 90% after administration of rAAV compared to the level of the protein of interest m before administration of rAAV. For example, the level of the protein of interest is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of rAAV compared to the level of the protein of interest before administration of rAAV.

In any of the methods, combination therapies or uses for treating a muscular dystrophy, the serum CK level in the subject is decreased after administration of the rAAV as compared to serum CK level before administration of the rAAV. For example, the serum CK level in the subject is decreased by about 65% to about 90% or about 65% to about 95% or about 75% to about 90% or about 80% to about 90% or about 85% to about 95% or about 87% to about 95% or about 87% to about 90% by 60 days after administration of the rAAV as compared to the serum CK level before administration of the rAAV. In particular, in any of the methods, combination therapies or uses for treating a muscular dystrophy of the disclosure, the serum CK level in the subject is decreased by about 87% by 60 days after administration of the rAAV as compared to the serum CK level before administration of the rAAV or in any of the methods, combination therapies or uses for of treating a muscular dystrophy of the disclosure, the serum CK level in the subject is decreased by about 72% by 60 days after administration of the rAAV as compared to the serum CK level before administration of the rAAV, or in any of the methods, combination therapies or uses for treating a muscular dystrophy of the disclosure, the serum CK level in the subject is decreased by about 73% by 60 days after administration of the rAAV as compared to the serum CK level before administration of the rAAV, or in any of the methods, combination therapies or uses for treating a muscular dystrophy of the disclosure, the serum CK level in the subject is decreased by about 78% by 60 days after administration of the rAAV as compared to the serum CK level before administration of the rAAV or in any of the methods, combination therapies or uses for treating a muscular dystrophy of the disclosure, the serum CK level in the subject is decreased by about 95% by 60 days after administration of the rAAV as compared to the serum CK level before administration of the rAAV. In any of the methods, combination therapies or uses for treating a muscular dystrophy, the number muscle fibers positive of the protein of interest in the muscle tissue of the subject is increased after administration of the rAAV as compared to the number of muscle fibers positive of the protein of interest before administration of the rAAV. For example, the number of muscle fibers positive of the protein of interest is detected by measuring the muscle fibers positive of the protein of interest level by Western blot or immunohistochemistry on muscle biopsies before and after administration of the rAAV.

In any of the methods, combination therapies or uses for treating a muscular dystrophy, administration of the rAAV upregulates expression of DAPC proteins such as alpha-sarcoglycan or beta-sarcoglycan. For example, the level of alpha-sarcoglycan in the subject is increased after administration of the rAAV as compared to the level of alpha-sarcoglycan before administration of the rAAV. In addition, the level of beta-sarcoglycan in the subject is increased after administration of the rAAV as compared to the level of the beta-sarcoglycan before administration of the rAAV. The level of alpha-sarcoglycan or beta-sarcoglycan is detected by measuring the alpha-sarcoglycan or beta-sarcoglycan protein level by Western blot or immunohistochemistry on muscle biopsies before and after administration of the rAAV.

In any of the methods, combination therapies or uses for treating a muscular dystrophy, disease progression in the subject is delayed after administration of the rAAV as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, in any of the methods, combination therapies or uses, the subject has at least a 6-point improvement in NSAA score at least 270 days after administration of the rAAV as compared to NSAA score before administration of the rAAV. Further, in any of the methods, combination therapies or uses, the subject has at least about 0.8 second improvement in time to rise at least 270 days after administration of the rAAV as compared to time to rise before administration of the rAAV. In addition, in any of the methods, combination therapies or uses, the subject has at least about 1.2 second improvement in time to ascend 4 steps test at least 270 days after administration of the rAAV as compared to time to ascend 4 steps test before administration of the rAAV. In addition, in any of the methods, combination therapies or uses, the subject has at least about 7 second improvement in the 100 m timed test at least 270 days after administration of the rAAV as compared to the 100 m timed test before administration of the rAAV.

In another embodiment, the disclosure provides for methods, compositions, combination therapies or uses for expressing micro-dystrophin gene in a patient cell comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6. For example, expression of the micro-dystrophin gene in the patient cell is detected by measuring the micro-dystrophin protein level by Western blot or immunohistochemistry in muscle biopsies before and after administration of the rAAV.MHCK7.micro-dystrophin construct. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In a further embodiment, the disclosure provides for methods, compositions, combination therapies or uses for decreasing serum CK levels in a patient in need thereof, the method comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the serum CK level in the patient is decreased by at least about 65% to about 90% or about 65% to about 95% or about 75% to about 90% or about 80% to about 90% or about 85% to about 95% or about 87% to about 95% or about 87% to about 90% by 60 days after administration of the rAAV as compared to the serum CK level before administration of the rAAV. In particular, the serum CK level in the subject is decreased by about 87% by 60 days after administration of the rAAV as compared to the serum CK level before administration of the rAAV or in any of the methods, compositions, combination therapies or uses for treating a muscular dystrophy of the disclosure, the serum CK level in the subject is decreased by about 72% by 60 days after administration of the rAAV as compared to the serum CK level before administration of the rAAV, or in any of the methods, compositions, combination therapies or uses for treating a muscular dystrophy of the disclosure, the serum CK level in the subject is decreased by about 73% by 60 days after administration of the rAAV as compared to the serum CK level before administration of the rAAV, or in any of the methods, compositions, combination therapies or uses for treating a muscular dystrophy of the disclosure, the serum CK level in the subject is decreased by about 78% by 60 days after administration of the rAAV as compared to the serum CK level before administration of the rAAV, or in any of the methods, compositions, combination therapies or uses for treating a muscular dystrophy of the disclosure, the serum CK level in the subject is decreased by about 95% by 60 days after administration of the rAAV as compared to the serum CK level before administration of the rAAV.

The disclosure also provides for methods, compositions, combination therapies or uses for increasing micro-dystrophin positive fibers in a patient muscle tissue comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the number of micro-dystrophin positive fibers is detected by measuring the dystrophin protein level by Western blot or immunohistochemistry on muscle biopsies before and after administration of the rAAV. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In another embodiment, the disclosure provides for methods, compositions, combination therapies or uses for increasing the expression of alpha-sarcoglycan in a patient in need thereof comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the level of alpha-sarcoglycan is detected by measuring the alpha-sarcoglycan protein level by Western blot or immunohistochemistry on muscle biopsies before and after administration of the rAAV.

In addition, the disclosure provides for methods, compositions, combination therapies or uses for increasing the expression of beta-sarcoglycan in a patient in need thereof comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the level of beta-sarcoglycan is detected by measuring the beta-sarcoglycan protein level by Western blot or immunohistochemistry on muscle biopsies before and after administration of the rAAV.

The disclosure also provides for methods, compositions, combination therapies or uses for treating a patient with Duchenne muscular dystrophy or Becker muscular dystrophy comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6, such that disease progression in the patient is delayed as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, in any of the methods, compositions, combination therapies or uses, the subject has at least a 6-point improvement in NSAA score at least 270 days after administration of the rAAV as compared to NSAA score before administration of the rAAV. Further, in any of the methods, the subject has at least about 0.8 second improvement in time to rise at least 270 days after administration of the rAAV as compared to time to rise before administration of the rAAV. In addition, in any of the methods, the subject has at least about 1.2 second improvement in time to ascend 4 steps test at least 270 days after administration of the rAAV as compared to time to ascend 4 steps test before administration of the rAAV. In addition, in any of the methods, the subject has at least about 7 second improvement in the 100 m timed test at least 270 days after administration of the rAAV as compared to the 100 m timed test before administration of the rAAV.

“Fibrosis” refers to the excessive or unregulated deposition of extracellular matrix (ECM) components and abnormal repair processes in tissues upon injury, including skeletal muscle, cardiac muscle, liver, lung, kidney, and pancreas. The ECM components that are deposited include fibronectin and collagen, e.g. collagen 1, collagen 2 or collagen 3.

The disclosure also provides for methods of reducing or preventing fibrosis in a subject suffering from muscular dystrophy comprising administering a therapeutically effective amount of a rAAV comprising the human micro-dystrophin nucleotide sequence of SEQ ID NO: 1 and the MHCK7 promoter nucleotide sequence of SEQ ID NO: 2 or SEQ ID NO: 7; or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MHCK7.microdystrophin. In one embodiment, the AAVrh74.MHCK7.microdystrophin is the AAVrh74.MHCK7.microdystrophin of nucleotides 55-5021 of SEQ ID NO: 3. In another embodiment, the AAVrh74.MHCK7.microdystrophin is the AAVrh74.MHCK7.microdystrophin of SEQ ID NO: 9. In another embodiment, the AAVrh74.MHCK7.microdystrophin is the AAVrh74.MHCK7.microdystrophin of nucleotides 1-4977 of SEQ ID NO: 8 or nucleotides 56-5066 of SEQ ID NO: 6. In a further embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In another embodiment, the disclosure provides for methods of preventing fibrosis in a subject in need thereof, comprising administering a therapeutically effective amount of the human micro-dystrophin nucleotide sequence of SEQ ID NO: 1 and the MHCK7 promoter nucleotide sequence of SEQ ID NO: 2 or SEQ ID NO:7; or rAAV vector comprising the AAV74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, any of the rAAV of the disclosure can be administered to subjects suffering from muscular dystrophy to prevent fibrosis, e.g. the rAAV of the disclosure expressing a human micro-dystrophin protein administered before fibrosis is observed in the subject. In addition, the rAAV of the disclosure expressing a human micro-dystrophin gene can be administered to a subject at risk of developing fibrosis, such as those suffering or diagnosed with muscular dystrophy, e.g. DMD. The rAAV of the disclosure can be administered to the subject suffering from muscular dystrophy in order to prevent new fibrosis in these subjects.

The disclosure contemplates administering rAAV before fibrosis is observed in the subject. In addition, the rAAV can be administered to a subject at risk of developing fibrosis, such as those suffering or diagnosed with a muscular dystrophy, e.g. DMD. The rAAV can be administered to the subject suffering from muscular dystrophy who already has developed fibrosis in order to prevent new fibrosis in these subjects.

The disclosure also provides for methods of increasing muscular force and/or muscle mass in a subject suffering from a muscular dystrophy comprising administering a therapeutically effective amount of the human micro-dystrophin nucleotide sequence of SEQ ID NO: 1 and the MHCK7 promoter nucleotide sequence of SEQ ID NO: 2 or SEQ ID NO: 7; or a rAAV comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

The disclosure contemplates administering rAAV vectors to subjects diagnosed with DMD before fibrosis is observed in the subject or before the muscle force has been reduced or before the muscle mass has been reduced.

The disclosure also contemplates administering the human micro-dystrophin nucleotide sequence of SEQ ID NO: 1 and the MHCK7 promoter nucleotide sequence of SEQ ID NO: 2 or SEQ ID NO:7; or a rAAV comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 to a subject suffering from a muscular dystrophy who already has developed fibrosis, in order to prevent new fibrosis in these subjects or to reduce fibrosis in these subjects. The disclosure also provides for administering the human micro-dystrophin nucleotide sequence of SEQ ID NO: 1 and the MHCK7 promoter nucleotide sequence of SEQ ID NO: 2 or SEQ ID NO:7; or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 to the subject suffering from a muscular dystrophy who already has reduced muscle force or has reduced muscle mass in order to protect the muscle from further injury.

In any of the methods of the disclosure, the subject may be suffering from a muscular dystrophy such as DMD or any other dystrophin-associated muscular dystrophy.

In other embodiments of any of the methods of the disclosure described herein, the serum CK level in the subject is decreased after administration of the rAAV as compared to the serum CK level before administration of the rAAV by a percentage level selected from the group consisting of:

a) at least 78% by 90, 180, or 270 days after the administration;
b) at least 46, 55, 56, 57, 58, 59, 60, 61, 62, 63, 64, 65, 66, 67, 68, 69, 70 or 85% by 270 days after the administration;
c) at least 72, 73, 74, or 95% by 180 days after the administration;
d) at least 87, 88, 93 or 95% by 90 days after the administration;
e) at least 70% by 270 days after the administration;
f) 70 to 95% by 90, 180, or 270 days after the administration;
g) at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In another embodiment, the disclosure provides for compositions for treating a muscular dystrophy in a human subject in need, wherein the composition comprises a dose of recombinant adeno-virus associated (rAAV) rAAV.MHCK7.microdystrophin, wherein composition is formulated for a systemic route of administration and the dose of the rAAV is about 1×10¹⁴ vg/kg to about 4×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

For example, the composition of the disclosure comprises a dose of rAAV of about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In one embodiment, the compositions of the disclosure are formulated for intravenous administration and comprise a dose of rAAV that is about 2.0×10¹⁴ vg/kg. In another embodiment, the compositions of the disclosure are formulated for intravenous administration and comprise a dose of rAAV that is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In another embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the compositions of the disclosure, the dose of rAAV is delivered in about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/kg, or about 10 mL/kg to 15 mL/kg or 10 mL/kg to about 20 mL/kg. In a particular embodiment, the composition comprises a dose of the rAAV delivered in about 10 mL/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In another embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

The compositions of the disclosure are formulated for administration by injection, infusion or implantation. For example, the compositions are formulated for administration by infusion over approximately one hour. In addition, the compositions of the disclosure are formulated for intravenous administration through a peripheral limb vein such as a peripheral arm vein or a peripheral leg vein. Alternatively, the infusion may be administered over approximately 30 minutes, or approximately 1.5 hours, or approximately 2 hours, or approximately 2.5 hours or approximately 3 hours.

Any of the compositions of the disclosure comprise a rAAV comprising the human micro-dystrophin nucleotide sequence of SEQ ID NO: 1 and the MHCK7 promoter sequence of SEQ ID NO: 2 or SEQ ID NO:7 or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In particular, the compositions of the disclosure are for treating Duchenne muscular dystrophy or Becker's muscular dystrophy. For example, the disclosure provides for compositions for treating Duchenne muscular dystrophy or Becker's muscular dystrophy in a human subject in need thereof wherein the composition comprises a dose of recombinant adeno-virus associated (rAAV) rAAV.MHCK7.microdystrophin, wherein the composition is formulated for administration by intravenous infusion over approximately one hour and the dose of the rAAV administered is about 2×10¹⁴ vg/kg, and wherein the rAAV comprises the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In another embodiment, the disclosure also provides a composition comprising rAAV for reducing fibrosis in a subject in need thereof. In addition, the disclosure provides a composition comprising a rAAV vectors for preventing fibrosis in a subject suffering from a muscular dystrophy.

The disclosure also provides for compositions comprising rAAV for increasing muscular force and/or muscle mass in a subject suffering from a muscular dystrophy. In a further embodiment, the disclosure provides for compositions comprising any of the rAAV of the disclosure for treatment of muscular dystrophy.

In other embodiments of any of the compositions of the disclosure, after administration of said composition to a human subject in need of treatment for muscular dystrophy, the serum CK level in the subject is decreased as compared to the serum CK level before administration of the composition by a percentage level selected from the group consisting of:

a) at least 78% by 90, 180, or 270 days after the administration;
b) at least 46, 55, 70, or 85% by 270 days after the administration;
c) at least 72, 73, 74, or 95% by 180 days after the administration;
d) at least 87, 99, 93 or 95% by 90 days after the administration;
e) at least 70% by 270 days after the administration;
f) 70 to 95% by 90, 180, or 270 days after the administration;
g) at least 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In another embodiment, the disclosure provides for use of a dose of recombinant adeno-virus associated (rAAV) rAAV.MHCK7.microdystrophin for the preparation of a medicament for the treatment of muscular dystrophy in a human subject in need thereof, wherein the medicament is formulated for a systemic route of administration and the dose of the rAAV is about 1×10¹⁴ vg/kg to about 4×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

For example, the medicament comprises a dose of rAAV of about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In one embodiment, the medicaments of the disclosure are formulated for systemic administration of a dose of rAAV wherein the systemic route of administration is an intravenous route and the dose of the rAAV administered is about 2.0×10¹⁴ vg/kg. In another embodiment, the medicament of the disclosure is formulated for systemic administration of a dose of rAAV wherein the systemic route of administration is an intravenous route and the dose of the rAAV is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the uses of the disclosure, the medicament comprises a dose of rAAV in about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/k, or about 10 mL/kg to 15 mL/kg or 10 mL/kg to about 20 mL/kg. In a particular embodiment, the dose or the rAAV is in about 10 mL/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the uses of the disclosure, the medicament is formulated for administration by injection, infusion or implantation. For example, the medicament is formulated for administration by infusion over approximately one hour. In addition, the medicament is formulated for intravenous administration through a peripheral limb vein, such as a peripheral arm vein or a peripheral leg vein. Alternatively, the infusion may be administered over approximately 30 minutes, or approximately 1.5 hours, or approximately 2 hours, or approximately 2.5 hours or approximately 3 hours.

In any of the uses of the disclosure, the medicament comprises an rAAV comprising the human micro-dystrophin nucleotide sequence of SEQ ID NO: 1 and the MHCK7 promoter sequence of SEQ ID NO: 2 or SEQ ID NO:7 or the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

A particular use of the disclosure is for preparation of a medicament for the treatment of Duchenne muscular dystrophy or Becker's muscular dystrophy. For example, the disclosure provides for use of a dose of recombinant adeno-virus associated (rAAV) rAAV.MHCK7.microdystrophin for the preparation of a medicament for treating Duchenne muscular dystrophy in a or Becker's muscular dystrophy human subject in need thereof, wherein the medicament is formulated for administration by intravenous infusion over approximately one hour and the dose of the rAAV administered is about 2×10¹⁴ vg/kg, and wherein the rAAV comprises the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In a further embodiment, the disclosure provides for use of a rAAV for preparation of a medicament for reducing fibrosis in a subject in need thereof. For example, the subject in need can be suffering from a muscular dystrophy, such as DMD or any other dystrophin associated muscular dystrophy.

In another embodiment, the disclosure provides for use of a rAAV for the preparation of a medicament to prevent fibrosis in a subject suffering from a muscular dystrophy.

In addition, the disclosure provides for use of a rAAV for the preparation of a medicament to increase muscular strength and/or muscle mass in a subject suffering from muscular dystrophy.

The disclosure also provides for use of the rAAV for the preparation of a medicament for treatment of muscular dystrophy.

The disclosure provides for use of a rAAV vector comprising the human micro-dystrophin nucleotide sequence of SEQ ID NO: 1 and the MHCK7 promoter nucleotide sequence of SEQ ID NO: 2 or SEQ ID NO:7 for preparation of a medicament for the treatment of a muscular dystrophy or a rAAV vector comprising the AAVrf74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for treatment of muscular dystrophy.

In other embodiments of any of the uses of the disclosure, the serum CK level in the subject is decreased after administration of the rAAV to the subject as compared to the serum CK level before administration of the rAAV by a percentage level selected from the group consisting of:

a) at least 78% by 90, 180, or 270 days after the administration;
b) at least 46, 55, 70, or 95% by 270 days after the administration;
c) at least 72, 73, 74, or 95% by 180 days after the administration;
d) at least 87, 88, 93 or 95% by 90 days after the administration;
e) at least 70% by 270 days after the administration;
f) 70 to 95% by 90, 180, or 270 days after the administration;
g) at least 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In any of the combination therapies, compositions for treating a muscular dystrophy or the uses of a medicament for treating a muscular dystrophy, the level of micro-dystrophin gene expression in a cell of the subject is increased after administration of the composition or medicament. Expression of the micro-dystrophin gene in the cell is detected by measuring the micro-dystrophin protein level by Western blot in muscle biopsied before and after administration of the composition or medicament. In particular, the level of micro-dystrophin protein is increased by at least about 70% to at least about 80%, or at least about 70% to at least about 90%, or at least about 80% to at least about 90% after administration of the composition or medicament compared to the level of micro-dystrophin before administration of the composition or medicament. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of the composition compared to the level of micro-dystrophin before administration of the composition or medicament.

In addition, expression of the micro-dystrophin gene in the cell is detected by measuring the micro-dystrophin protein level by immunohistochemistry in muscle biopsies before and after administration of the composition or medicament. The level of micro-dystrophin protein is increased by at least about 70% to at least about 80%, or at least about 70% to at least about 90%, or at least about 80% to at least about 90% after administration of rAAV compared to the level of micro-dystrophin before administration of the composition or medicament. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of the composition or medicament compared to the level of micro-dystrophin before administration of the composition or medicament.

In any of the combination therapies, compositions for treating a muscular dystrophy and uses of a medicament for treating muscular dystrophy, the serum CK level in the subject is decreased after administration of the rAAV as compared to serum CK level before administration of the composition or medicament. For example, the serum CK level in the subject is decreased by about 65% to about 90% or about 65% to about 95% or about 75% to about 90% or about 80% to about 90% or about 85% to about 95% or about 87% to about 95% or about 87% to about 90% by 60 days after administration of the rAAV as compared to the serum CK level before administration of the composition or medicament. In particular, in any of the combination therapies, compositions and uses of a medicament for treating a muscular dystrophy of the disclosure, the serum CK level in the subject is decreased by about 87% by 60 days after administration rAAV as compared to the serum CK level before administration of the rAAV. In any of the combination therapies, compositions for treating a muscular dystrophy or the uses of a medicament for treating a muscular dystrophy of the disclosure, the serum CK level in the subject is decreased by about 72% by 60 days after administration of the rAAV as compared to the serum CK level before administration of the combination therapy, composition or medicament, or in any of the combination therapies, compositions for treating a muscular dystrophy or medicaments for treating a muscular dystrophy of the disclosure, the serum CK level in the subject is decreased by about 73% by 60 days after administration of the rAAV as compared to the serum CK level before administration of the rAAV, or in any of the compositions for treating a muscular dystrophy or the uses of a medicament for treating a muscular dystrophy of the disclosure, the serum CK level in the subject is decreased by about 78% by 60 days after administration of the rAAV as compared to the serum CK level before administration of the combination therapy, composition or medicament or in any of the combination therapies, compositions for treating a muscular dystrophy or the uses of a medicament for treating a muscular dystrophy of the disclosure, the serum CK level in the subject is decreased by about 95% by 60 days after administration of the rAAV as compared to the serum CK level before administration of the combination therapy, composition or medicament. In any of the combination therapies, composition for treating a muscular dystrophy or the uses of a medicament for treating a muscular dystrophy, the number of micro-dystrophin positive fibers in the muscle tissue of the subject is increased after administration of the combination therapy, composition or medicament as compared to the number of micro-dystrophin positive fibers before administration of the rAAV. For example, the number of micro-dystrophin positive fibers is detected by measuring the micro-dystrophin protein level by Western blot or immunohistochemistry on muscle biopsies before and after administration of the combination therapy, composition or medicament.

In any of the combination therapies, compositions for treating a muscular dystrophy or the uses of a medicament for treating a muscular dystrophy, administration of the composition or medicament upregulates expression of DAPC proteins such as alpha-sarcoglycan or beta-sarcoglycan. For example, the level of alpha-sarcoglycan in the subject is increased after administration of the rAAV as compared to the level of alpha-sarcoglycan before administration of the combination therapy, composition or medicament. The level of alpha-sarcoglycan or beta-sarcoglycan is detected by measuring the alpha-sarcoglycan or beta-sarcoglycan protein level by Western blot or immunohistochemistry on muscle biopsies before and after administration of the rAAV.

In any of the combination therapies, compositions for treating a muscular dystrophy or the uses of a medicament for treating a muscular dystrophy, disease progression in the subject is delayed after administration of the rAAV as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, after administration of any of the combination therapies, compositions for treating a muscular dystrophy or the uses of a medicament for treating a muscular dystrophy, the subject has at least a 6-point improvement in NSAA score at least 270 days after administration of the composition or medicament as compared to NSAA score before administration of the rAAV. Further, in any of the methods, combination therapies, compositions for treating a muscular dystrophy or the uses of a medicament for treating a muscular dystrophy, the subject has at least about 0.8 second improvement in time to rise at least 270 days after administration of the rAAV as compared to time to rise before administration of the combination therapy, composition or medicament. In addition, in any of the methods, combination therapies, compositions or uses of the disclosure, the subject has at least about 1.2 second improvement in time to ascend 4 steps test at least 270 days after administration of the rAAV as compared to time to ascend 4 steps test before administration of the combination therapy, composition or medicament. In addition, in any of the methods, combination therapy, compositions or uses of the disclosure, the subject has at least about 7 second improvement in the 100 m timed test at least 270 days after administration of the rAAV as compared to the 100 m timed test before administration of the rAAV.

In another embodiment, the disclosure provides for compositions for expressing micro-dystrophin gene in a patient cell comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6. In a further embodiment, the disclosure provides for use of a dose of a AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6 for the preparation of a medicament for expressing micro-dystrophin gene in a patient cell. For example, expression of the micro-dystrophin gene in the patient cell is detected by measuring the micro-dystrophin protein level by Western blot or immunohistochemistry in muscle biopsies before and after administration of the rAAV.MHCK7.micro-dystrophin construct. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In a further embodiment, the disclosure provides for compositions for decreasing serum CK levels in a patient in need thereof, the composition comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In addition, the disclosure provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for decreasing serum CK levels in a patient in need thereof. For example, the serum CK level in the patient is decreased by at least about 65% to about 90% or about 65% to about 95% or about 75% to about 90% or about 80% to about 90% or about 85% to about 95% or about 87% to about 95% or about 87% to about 90% by 60 days after administration of the composition or medicament as compared to the serum CK level before administration of the composition or medicament. In particular, the serum CK level in the subject is decreased by about 87% by 60 days after administration of the composition or medicament as compared to the serum CK level before administration of the composition or medicament, or decreased by about 72% by 60 days after administration of the composition or medicament as compared to the serum CK level before administration of the composition or medicament, or decreased by about 73% by 60 days after administration of the composition or medicament as compared to the serum CK level before administration of the composition or medicament, or decreased by about 78% by 60 days after administration of the composition or medicament as compared to the serum CK level before administration of the composition or medicament, or decreased by about 95% by 60 days after administration of the composition or medicament as compared to the serum CK level before administration of the composition or medicament.

The disclosure also provides for compositions for increasing micro-dystrophin positive fibers in a patient muscle tissue comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In addition, the disclosure provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing micro-dystrophin positive fibers in a patient muscle tissue. For example, the number of micro-dystrophin positive fibers is detected by measuring the dystrophin protein level by Western blot or immunohistochemistry on muscle biopsies before and after administration of the composition or medicament. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In another embodiment, the disclosure provides for compositions for increasing the expression of alpha-sarcoglycan in a patient in need thereof comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. The disclosure also provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing the expression of alpha-sarcoglycan in a patient in need thereof. For example, the level of alpha-sarcoglycan is detected by measuring the alpha-sarcoglycan protein level by Western blot or immunohistochemistry on muscle biopsies before and after administration of the composition or medicament.

In addition, the disclosure provides for compositions for increasing the expression of beta-sarcoglycan in a patient in need thereof comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. The disclosure also provides for use of the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing the expression of beta-sarcoglycan in a patient in need thereof. For example, the level of beta-sarcoglycan is detected by measuring the beta-sarcoglycan protein level by Western blot or immunohistochemistry on muscle biopsies before and after administration of the composition or medicament.

The disclosure also provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for treating a patient with Duchenne muscular dystrophy or Becker muscular dystrophy, such that administration of the medicament results in disease progression in the patient is delayed as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, the subject has at least a 6-point improvement in NSAA score at least 270 days after administration of the rAAV as compared to NSAA score before administration of the rAAV. Further, the subject has at least about 0.8 second improvement in time to rise at least 270 days after administration of the rAAV as compared to time to rise before administration of the rAAV. In addition, the subject has at least about 1.2 second improvement in time to ascend 4 steps test at least 270 days after administration of the rAAV as compared to time to ascend 4 steps test before administration of the composition or medicament. In addition, the subject has at least about 7 second improvement in the 100 m timed test at least 270 days after administration of the rAAV as compared to the 100 m timed test before administration of the composition or medicament.

### BRIEF DESCRIPTION OF THE DRAWINGS

FIG. 1 illustrates the rAAV.MHCK7.micro-dystrophin construct. In this construct, the cDNA expression cassette is flanked by AAV2 inverted terminal repeat sequences (ITR). The construct is characterized by an in-frame rod deletion (R4-R23), while hinges 1, 2 and 4 (H₁, H₂ and H₄) and the cysteine rich domain remain producing a 138 kDa protein. The expression of the micro-dystrophin protein (3579 bp) is guided by a MHCK7 promoter (795 bp). The intron and 5′ UTR are derived from plasmid pCMVB (Clontech). The micro-dystrophin cassette had a consensus Kozak immediately in front of the ATG start and a small 53 bp synthetic polyA signal for mRNA termination. The human micro-dystrophin cassette contained the (R4-R23/Δ71-78) domains as previously described by Harper et al. (Nature Medicine 8, 253-261 (2002)).

FIGS. 2A-2D provide the nucleic acid sequence (SEQ ID NO: 3) rAAVrh74.MHCK7.micro-dystrophin.

FIG. 3 provides the pNLREP2-Caprh74 AAV helper plasmid map.

FIG. 4 provides the Ad Helper plasmid pHELP.

FIG. 5 illustrates the rAAV.MCK.micro-dystrophin plasmid construct.

FIGS. 6A-6D provide the nucleic acid sequence (SEQ ID NO: 5) rAAVrh74.MCK.micro-dystrophin.

FIGS. 7A-7C provide the nucleic acid sequence (SEQ ID NO: 9) rAAVrh74.MHCK7.micro-dystrophin.

FIG. 8 illustrates the AAVrh74.MHCK7.micro-dystrophin plasmid construct.

FIGS. 9A-9D provide the nucleic acid sequence (SEQ ID NO: 8) of the rAAVrh74.MHCK7.micro-dystrophin plasmid construct, which comprises kanamycin resistance gene.

FIG. 10 provides a schematic of therapeutic β-sarcoglycan transgene cassette. Self-complementary AAV vector containing the codon-optimized human β-sarcoglycan gene (hSGCB). A muscle specific MHCK7 promoter drives expression. The cassette also contains a chimeric intron to augment processing and polyadenylation signal for stability.

FIG. 11 provides a graph of antibody titer to AAVrh74 in NHPs following re-dosing with rAAVrh74.MHCK7.micro-dystrophin. The dotted line represents inclusion criteria for total AAVrh.74 antibody titer levels which was a threshold of 1:400 against AAVrh.74.

FIG. 12 provides the fold change in micro-dystrophin protein expression post-TPE compared to before TPE in NHPs redosed with rAAVrh74.MHCK7.micro-dystrophin after TPE.

FIG. 13 provide a course of plasma antibodies following plasma volume (PV) removal over a 10-day course of TPE.

### DETAILED DESCRIPTION

The present disclosure is directed to identifying the dose, duration, and immunosuppression regimen for enhancing gene expression after intravascular delivery of rAAV.rh74.MHCK7.micro-dystrophin; to identifying techniques and enhancing gene expression after dosing or re-dosing with the rAAV in conjunction with using TPE to remove pre-existing AAV antibodies; and to evaluate redosing without the use of TPE. The methods of the invention encompass administering an immunosuppression regimen and/or TPE prior to any dosing with rAAV.rh74.MHCK7.micro-dystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, or rAAVrh.74.MHCK7.huAN05 as described herein; and the methods encompass administering an immunosuppression regimen and/or TPE prior to re-dosing with rAAV.rh74.MHCK7.micro-dystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, or rAAVrh.74.MHCK7.huAN05 as described herein.

The present disclosure provides for gene therapy vectors, e.g. rAAV vectors, overexpressing human micro-dystrophin and methods of reducing and preventing fibrosis in muscular dystrophy patients. Muscle biopsies taken at the earliest age of diagnosis of DMD reveal prominent connective tissue proliferation. Muscle fibrosis is deleterious in multiple ways. It reduces normal transit of endomysial nutrients through connective tissue barriers, reduces the blood flow and deprives muscle of vascular-derived nutritional constituents, and functionally contributes to early loss of ambulation through limb contractures. Over time, treatment challenges multiply as a result of marked fibrosis in muscle. This can be observed in muscle biopsies comparing connective tissue proliferation at successive time points. The process continues to exacerbate leading to loss of ambulation and accelerating out of control, especially in wheelchair-dependent patients.

Without early treatment including a parallel approach to reduce fibrosis it is unlikely that the benefits of exon skipping, stop-codon read-through, or gene replacement therapies can ever be fully achieved. Even small molecules or protein replacement strategies are likely to fail without an approach to reduce muscle fibrosis. Previous work in aged mdx mice with existing fibrosis treated with AAV.micro-dystrophin demonstrated that we could not achieve full functional restoration (Liu, M., et al., Mol Ther 11, 245-256 (2005)). It is also known that progression of DMD cardiomyopathy is accompanied by scarring and fibrosis in the ventricular wall.

The practice of the present invention will employ, unless otherwise indicated, conventional methods of virology, microbiology, molecular biology and recombinant DNA techniques within the skill of the art. Such techniques are explained fully in the literature. See, e.g., Sambrook et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.); Freshney Culture of Animal Cells, A Manual of Basic Technique (Wiley-Liss, Third Edition); and Ausubel et al. (1991) Current Protocols in Molecular Biology (Wiley Interscience, N.Y.).

All publications, patents and patent applications cited herein, whether supra or infra, are hereby incorporated by reference in their entirety.

### Definitions

As used herein, the singular forms “a,” “an,” and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. Reference to “a recombinant AAV” includes a mixture of two or more rAAV virions, and the like. All technical and scientific terms used herein have the same meaning as commonly understood to one of ordinary skill in the art to which this invention belongs unless clearly indicated otherwise.

The use of the term “or” in the claims is used to mean “and/or” unless explicitly indicated to refer to alternatives only, or the alternatives are mutually exclusive, although the disclosure supports a definition that refers to only alternatives and “and/or.”

Throughout this application, the term “about” is used to indicate that a value includes the statistical experimental error (standard deviation of error) for the device or method being employed to determine the value.

The term “vector” is meant to be any genetic element, such as a plasmid, phage, transposon, cosmid, chromosome, virus, virion, etc., which is capable of replication when associated with the proper control elements and which can transfer gene sequences between cells. In one embodiment, the vector is a viral vector.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products. In one embodiment, the AAV vector is a vector derived from an adeno-associated virus serotype, including without limitation, AAV-1, AAV-2, AAV-3, AAV-4, AAV-5, AAV-6, AAV-7, AAV-8, AAV-9, AAV-10, AAV-11, AAV-12, AAV-13, AAV rh10, and AAVrh.74. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, as long as the sequences provide for functional rescue, replication and packaging.

The term “AAV helper functions” refer to AAV-derived coding sequences that can be expressed to provide AAV gene products that, in turn, function in trans for productive AAV replication. Thus, AAV helper functions comprise the major AAV open reading frames (ORFs), reps and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

By “recombinant virus” is meant a virus that has been genetically altered, e.g., by the addition or insertion of a heterologous nucleic acid sequence into the viral particle.

An “AAV virion” or “AAV viral particle” or “AAV vector particle” refers to a viral particle composed of at least one AAV capsid protein and an encapsidated polynucleotide AAV vector. The AAV virion, in one embodiment, comprises a heterologous polynucleotide (i.e. a polynucleotide other than a wild-type AAV genome such as a transgene to be delivered to a mammalian cell). Production of AAV viral particles, in some embodiment, includes production of AAV vector, as such a vector is contained within an AAV vector particle.

AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

For example, a wild-type (wt) AAV virus particle comprising a linear, single-stranded AAV nucleic acid genome associated with an AAV capsid protein coat. The AAV virion can be either a single-stranded (ss) AAV or self-complementary (SC) AAV. In one embodiment, a single-stranded AAV nucleic acid molecules of either complementary sense, e.g., “sense” or “antisense” strands, can be packaged into a AAV virion and both strands are equally infectious.

The term “recombinant AAV,” or “rAAV” is defined herein as an infectious, replication-defective virus composed of an AAV protein shell, encapsidating a heterologous nucleotide sequence of interest which is flanked on both sides by AAV ITRs. A rAAV, in one embodiment, is produced in a suitable host cell which has an AAV vector, AAV helper functions and accessory functions introduced therein. In this manner, the host cell is capable of encoding AAV polypeptides that are required for packaging the AAV vector (containing a recombinant nucleotide sequence of interest) into infectious recombinant virion particles for subsequent gene delivery.

The term “transfection” refers to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a nucleotide integration vector and other nucleic acid molecules, into suitable host cells.

The term “transduction” denotes the delivery of a DNA molecule to a recipient cell either in vivo or in vitro, via a replication-defective viral vector, such as via a recombinant AAV virion.

The term “host cell” denotes, for example, microorganisms, yeast cells, insect. cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “coding sequence” or a sequence which “encodes” a particular protein, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

A “nucleic acid” sequence refers to a DNA or RNA sequence. The nucleic acids include base analogues of DNA and RNA including, but not limited to 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyl adenine, 2-methylguanine, 3-methylcytosine, 5-methylcytosine, N6-methyladenine, 7-methylguanine, 5-methyl aminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters.” In one embodiment, the promoter is a muscle-specific promoter, which includes but is not limited to, a human skeletal actin gene element, a cardiac actin gene element, a desmin promoter, a skeletal alpha-actin (ASKA) promoter, a troponin I (TNNI2) promoter, a myocyte-specific enhancer binding factor mef binding element, a muscle creatine kinase (MCK) promoter, a truncated MCK (tMCK) promoter, a myosin heavy chain (MHC) promoter, a hybrid a-myosin heavy chain enhancer-/MCK enhancer-promoter (MHCK7) promoter, a C5-12 promoter, a murine creatine kinase enhancer element, a skeletal fast-twitch troponin c gene element, a slow-twitch cardiac troponin c gene element, a slow-twitch troponin i gene element, hypoxia-inducible nuclear factor (HIF)-response element (HIRE), a steroid-inducible element, and a glucocorticoid response element (gre). In another embodiment, the promoter is an MCK promoter, a tMCK promoter, or an MHCK7 promoter.

The term “operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

A promoter “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA, at least one multiple cloning site, and a “mammalian” origin of replication (e.g., a SV40 or adenovirus origin of replication).

By “isolated” when referring to a nucleotide sequence, is meant that the indicated molecule is present in the substantial absence of other biological macromolecules such as other nucleotide sequences, chromatin material, etc. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

For the purpose of describing the relative position of nucleotide sequences in a particular nucleic acid molecule throughout the instant application, such as when a particular nucleotide sequence is described as being situated “upstream,” “downstream,” “3,” or “5” relative to another sequence, it is to be understood that it is the position of the sequences in the “sense” or “coding” strand of a DNA molecule that is being referred to as is conventional in the art.

The terms “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. The percentage identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

The term “subject” refers to any member of the animal kingdom, which includes, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. In some embodiments, the subject is a human ranging in age from birth to 2 years, from 1 to 10 years, or ranging from 4 to 15 years, or ranging from 10 to 19 years, or from 20 to 40 years of age, or from 15 to 29 years of age or from 25-55 years, or ranging from 40 to 60 years, or over 50 years or over 60 years or over 65 years or over 70 years.

### AAV

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes AAVrh.74, AAV-1, AAV-2, AAV-3, AAV-4, AAV-5, AAV-6, AAV-7, AAV-8, AAV-9, AAV-10, AAV-11, AAV-12, AAV-13 and AAVrh.74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote muscle-specific expression, AAVrh.74 can be used.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samul ski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the disclosure comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the disclosure are set out in International Patent Application No. PCT/US2012/047999 (WO 2013/016352) incorporated by reference herein in its entirety.

In an exemplary embodiment, the recombinant AAV vector of the disclosure is produced by the triple transfection method (Xiao et al., J Virol 72, 2224-2232 (1998) using the AAV vector plasmids rAAV.MHCK7.micro-dystrophin, pNLRep2-Caprh74 and pHelp, rAAV contains the micro-dystrophin gene expression cassette flanked by AAV2 inverted terminal repeat sequences (ITR). It is this sequence that is encapsidated into AAVrh.74 virions. The plasmid contains the micro-dystrophin sequence and the MHCK7 enhancer and core promoter elements of the muscle specific promoter to drive gene expression. The expression cassette also contains an SV40 intron (SD/SA) to promote high-level gene expression and the bovine growth hormone polyadenylation signal is used for efficient transcription termination.

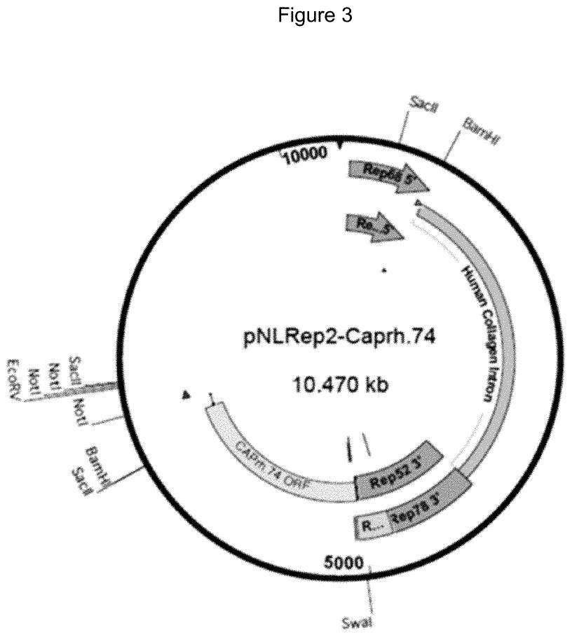

The pNLREP2-Caprh74 is an AAV helper plasmid that encodes the 4 wild-type AAV2 rep proteins and the 3 wild-type AAV VP capsid proteins from serotype rh74. A schematic map of the pNLREP2-Caprh74 plasmid is shown in FIG. 3.

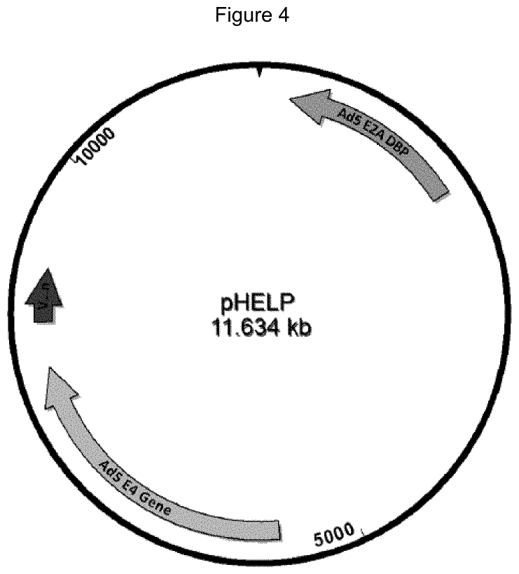

The pHELP adenovirus helper plasmid is 11,635 bp and was obtained from Applied Viromics. The plasmid contains the regions of adenovirus genome that are important for AAV replication, namely E2A, E4ORF6, and VA RNA (the adenovirus E1 functions are provided by the 293 cells). The adenovirus sequences present in this plasmid only represents ˜40% of the adenovirus genome, and does not contain the cis elements critical for replication such as the adenovirus terminal repeats. Therefore, no infectious adenovirus is expected to be generated from such a production system. A schematic map of the pHELP plasmid is shown in FIG. 4.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising rAAV of the present disclosure. Compositions of the disclosure comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed and include buffers and surfactants such as pluronics.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). One exemplary method of determining encapsilated vector genome titer uses quantitative PCR such as the methods described in (Pozsgai et al., Mol. Ther. 25(4): 855-869, 2017). Unless stated otherwise, the dosages described herein correspond to a dose as determined by the supercoiled DNA standard.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the disclosure is DMD.

Combination therapies are also contemplated by the disclosure. Combination as used herein includes both simultaneous treatment and sequential treatments. Combinations of methods of the disclosure with standard medical treatments (e.g., corticosteroids) are specifically contemplated, as are combinations with novel therapies.

Administration of an effective dose of the compositions, combination therapies or medicaments may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the micro-dystrophin protein.

The disclosure provides for local administration and systemic administration of an effective dose of rAAV, medicaments and compositions of the disclosure. For example, systemic administration is administration into the circulatory system so that the entire body is affected. Systemic administration includes enteral administration such as absorption through the gastrointestinal tract and parenteral administration through injection, infusion or implantation.

In particular, actual administration of rAAV of the present disclosure may be accomplished by using any physical method that will transport the rAAV recombinant vector into the target tissue of an animal. Administration according to the disclosure includes, but is not limited to, injection into muscle and injection into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

In one embodiment of the disclosure, the AAVrh74.MHCK7.microdystrophin described herein is formulated in a buffer containing 20 mM Tris (pH 8.0), 1 mM magnesium chloride (MgCl₂), 200 mM sodium chloride (NaCl), and 0.001% poloxamer 188.

The dose of rAAV to be administered in methods disclosed herein will vary depending, for example, on the particular rAAV, the mode of administration, the treatment goal, the individual, and the cell type(s) being targeted, and may be determined by methods standard in the art. Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 2×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg 1×10¹³ vg, 1×10¹⁴ vg, 2×10¹⁴ vg, 1×10¹⁵ vg respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1.25×10¹⁴ vg/kg, 1.5×10¹⁴ vg/kg, 1.75×10¹⁴ vg/kg, 2.0×10¹⁴ vg/kg, 2.25×10¹⁴ vg/kg, 2.5×10¹⁴ vg/kg, 2.75×10¹⁴ vg/kg, 3.0×10¹⁴ vg/kg, 3.25×10¹⁴ vg/kg, 3.5×10¹⁴ vg/kg, 3.75×10¹⁴ vg/kg, 4.0×10¹⁴ vg/kg, 1×10¹⁵ vg/kg respectively). Methods for titering AAV are described in Clark et al., Hum. Gene Ther., 10: 1031-1039 (1999).

In particular, actual administration of rAAV of the present disclosure may be accomplished by using any physical method that will transport the rAAV recombinant vector into the target tissue of an animal. Administration according to the disclosure includes, but is not limited to, injection into muscle and injected into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the disclosure results in sustained expression of the micro-dystrophin protein. The present disclosure thus provides methods of administering/delivering rAAV which express micro-dystrophin protein to an animal, preferably a human being. These methods include transducing tissues (including, but not limited to, tissues such as muscle, organs such as liver and brain, and glands such as salivary glands) with one or more rAAV of the present disclosure. Transduction may be carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family (See Weintraub et al., Science, 251: 761-766 (1991)), the myocyte-specific enhancer binding factor MEF-2 (Cserjesi and Olson, Mol Cell Biol 11: 4854-4862 (1991)), control elements derived from the human skeletal actin gene (Muscat et al., Mol Cell Biol, 7: 4089-4099 (1987)), the cardiac actin gene, muscle creatine kinase sequence elements (See Johnson et al., Mol Cell Biol, 9:3393-3399 (1989)) and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA, 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

Muscle tissue is an attractive target for in vivo DNA delivery, because it is not a vital organ and is easy to access. The disclosure contemplates sustained expression of microdystrophin from transduced myofibers.

Thus, the disclosure provides methods of administering an effective dose (or doses, administered essentially simultaneously or doses given at intervals) of rAAV that encode micro-dystrophin to a subject in need thereof.

### Immunosuppressing Regimen

The disclosure provides for methods of treating muscular dystrophy wherein the subject is undergoing an immunosuppressing regimen. The term immunosuppressing regimen refers to a method of treatment which suppresses or modulate the immune system of the subject. The regimen comprises administration of one or more immune suppressing agents. In any of the methods, the immunosuppressing regimen comprises at least one immune suppressing agent, or at least two immune suppressing agent or at least three immune suppressing agent or at least four immune suppressing agent or at least five suppressing agent.

The immunosuppressing regimen is administered prophylactically, in that the immunosuppressing regimen is administered prior to administration of the gene therapy, or prior to the onset of an immune response to the rAAV in the subject after administration of the gene therapy. The immune response includes an adverse immune response or an inflammatory response to the administered rAAV. The immune response may be the production of antibodies in the subject in response to the administered rAAV, such as anti-AAVrh.74 antibodies.

Prophylactic administration includes administration of the immunosuppressing regimen at the same time as administration of the gene therapy, such as within 24 hours of administration of the gene therapy, or within 12 hours of administration of the gene therapy, or within 6 hours of administration of the gene therapy, or within 5 hours of administration of the gene therapy, or within 4 hours of administration of the gene therapy, or within 3 hours of administration of the gene therapy, or within 2 hours of administration of the gene therapy or within our of administration of the gene therapy. The immune suppressing agent is any agent that inhibits the subject's immune system, reduces the effectiveness of the subject's immune system or modulates the activity or effectiveness of the subject's immune system.

In other embodiments, the immunosuppressing regimen is administered therapeutically. For example, the immunosuppressing regimen is administered after the onset of an immune response to the rAAV in the subject after administration of the gene therapy. The immune response in a subject includes an adverse immune response or an inflammatory response following or caused by the administration of rAAV to the subject. The immune response may be the production of antibodies in the subject in response to the administered rAAV, such as anti-AAVrh.74 antibodies.

In other embodiments, the immunosuppressing regimen is administered prior to administering a second dose of the gene therapy. In some embodiments, the second dose is administered after therapeutic plasma exchange (TPE).

Exemplary immune suppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors and biologics such as monoclonal antibodies or fusion proteins and polypeptides.

The immune suppressing agent may be an anti-inflammatory steroid, which is a steroid that decreases inflammation and suppresses or modulates the immune system of the subject. Exemplary anti-inflammatory steroid are glucocorticoids such as prednisone, prednisolone, betamethasone, dexamethasone, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineuine inhibitors includes cyclosporine, tacrolimus and picecrolimus.

mTOR inhibitors reduce or inhibit the serine/threonine-specific protein kinase mTOR. Exemplary mTOR inhibitors include sirolimus, everolimus, and temsirolimus.

The immune suppressing agents include immune suppressing macrolides. The term “immune suppressing macrolides” refer to macrolide agents that suppresses or modulates the immune system of the subject. A macrolide is a classes of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and sirolimus.

Purine analogs block nucleotide synthesis and include IMDH inhibitors. Exemplary purine analogs include azathioprine, mycophenolate and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immune suppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immune suppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

In embodiments of the invention, an immune suppressing agent utilized in one or more methods of the disclosure (immune suppressing antibodies), can be administered as a nanoparticle. Methods for making and/or formulating a nanoparticle, and nanoparticles that can be utilized in the methods of the disclosure, include those described in, for example, as polymers (Patil et al., Pharmaceutical Nanotechnol. 367:195-203, 2009; Yang et al., ACS Appl. Mater. Interfaces, doi: 10.1021/acsami.6b16556, 2017; Perepelyuk et al., Mol. Ther. Nucleic Acids 6:259-268, 2017); as liposomes (Buyens et al., J. Control Release 158(3): 362-370, 2012; Scarabel et al., Expert Opin. Drug Deliv. 17:1-14, 2017); as micelles (Tangsangasaksri et al., Bio Macromolecules 17:246-255, 2016; Wu et al., Nanotechnology, doi: 10.1088/1361-6528/aa6519, 2017); as microemulsion (WO 11/004395), as nanoemulsion or a solid lipid nanoparticle (Sahay et al., Nature Biotechnol. 31:653-658, 2013; and Lin et al., Nanomedicine 9(1):105-120, 2014); and those described in WO 2008/066965, WO 2011/143201, WO2008/014478, WO 2020/081938, WO 2013/016058, WO 2013/086373, WO 2019/177550, WO 2013/016126, WO 2019/089828, WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO 2011/141705 and WO 2001/07548; U.S. Patent Publication Nos. 2004/0142025, 2007/0042031, 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832,2012/0183581, 2012/0172411, 2012/0027803, 2012/0058188, 2011/0311583, 2011/0311582, 2011/0262527, 2011/0216622, 2011/0117125, 2011/0091525,2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076, and U.S. Pat. Nos. 8,569,256, 5,965,542; the entire contents of which is hereby incorporated herein by reference.

### Therapeutic Plasma Exchange

Therapeutic plasma exchange (TPE) is an extracorporal blood purification technique designed to remove high molecular weight substances, such as antibodies. After initial systemic administration of a dose of rAAV, the subject may develop antibodies to the AAV serotype, e.g. the subject develops antibodies to AAVrh74 after administration of rAAV.MHCK7.microdystrophin. Removing these antibodies using TPE allows for safe and efficacious re-dosing of a rAAV vector. In the TPE process, whole blood is removed via vascular access and subsequently spun through a centrifuge within the apheresis machine, where the plasma (antibodies) is removed. Red blood cells are delivered back along with a replacement fluid (Human Albumin) for maintenance fluid for the subject.

The amount of plasma to be exchanged in a TPE session is determined in relation to the subject's estimated plasma volume (EPV). A number of formulas may be used to calculate the EPV (see, e.g. Inkley et al., J. Lab Clin. Med. 45:841-850, 1955, Retzlaff et al., Blood 33: 649-887, 1969, Feldschuh et al., Circulation 56: 605-612, 1977, Spenger et al., Predication of patient's plasma volume in plasma exchange therapy. In: Smeby et al. ed. Immune and Metabolic Aspects of Therapeutic Blood Purification Systems. Basel, Switzerland: Krager. 1986, pp. 394-402). An exemplary method of estimating the EPV is calculated using the subject's weight and hematocrit according to the following formula which is described in Kaplan et al. Kidney Intl. 38:160-166, 1990, which is incorporated by reference herein in its entirety:

EPV=[0.065×weight (kg)]×[1−Hemocrit]

The protocol described in Example 5 was developed based on the kinetics of IgG antibodies, showing that after 48 hours there is rebound, gaining back roughly 50-60% of antibodies removed from the previous procedure. For the purposes of the methods described herein, it was determined that in one embodiment, antibody titer must be reduced to AAVrh.74 Antibody Level <1:100 to permit gene delivery.

For example, methods of subjecting a subject's plasma to TPE removes at least about 50% of anti-rAAV antibodies within the intravascular space of the subject, or removes at least about 55% of anti-rAAV antibodies within the intravascular space of the subject, or removes at least about 60% of anti-rAAV antibodies within the intravascular space of the subject, or removes at least about 63% of anti-rAAV antibodies, or removes at least about 64% of anti-rAAV antibodies within the intravascular space of the subject or removes at least about 65% of anti-rAAV antibodies within the intravascular space of the subject, or removes at least about 69% of anti-rAAV antibodies within the intravascular space of the subject or removes at least about 70% of anti-rAAV antibodies within the intravascular space of the subject, or removes at least about 74% of anti-rAAV antibodies within the intravascular space of the subject or removes at least about 75% of anti-rAAV antibodies within the intravascular space of the subject, or removes at least about 85% of anti-rAAV antibodies within the intravascular space of the subject, or removes at least about 90% of anti-rAAV antibodies, or removes at least about 95% of anti-rAAV antibodies within the intravascular space of the subject.

The disclosed methods comprising subjecting the subject's plasma to at least one TPE session, or at least two TPE sessions, or at least three TPE sessions, or at least 5 TPE sessions, or at least 6 TPE sessions, or at least 7 TPE sessions, or at least 8 TPE sessions, or at least 9 TPE sessions or at least 10 TPE sessions. In addition, the TPE sessions are carried out once a day over about 1 to 5 days, or about 1 to 10 days, or about 5 to 10 days, or about 5 to 7 days or about 7 to 10 days. The TPE sessions are carried out once a day for two consecutive days, or once a day for three consecutive days or once a day for four consecutive days for 5 consecutive days, or once a day for 6 consecutive days, or once a day for 7 consecutive days, or once a day for 7 consecutive days, or once a day for 8 consecutive days, or once a day for 9 consecutive days or once a day for 10 consecutive days.

The TPE is carried out with techniques are carried out using blood bank procedures using selective cell removal (cytopheresis) as described in Gurland et al., Int. J. Artif Organs 7:35-38, 1984, using membrane plasma separation (MPS) which uses a highly permeable filter and dialysis equipment as described in Gurland et al., Nephron 36:173-182, 1984. Additional methods of carrying out TPE are described in Sowada et al. (Available removal systems: state of the art. In Nydegger U E, editor. Therapeutic Hemapheresis in the 1990s. Current Studies in Hematology and Blood Transfusions, Vol 57. Basal Switzerland: Karger. 1990 pp. 57-113). These references are incorporated herein by reference in their entirety.

Embodiments of the invention include determination of the presence of anti-AAVrh.74 antibodies in serum or plasma of a subject in conjunction with treatment of the subject with a (rAAV) rAAV.MHCK7.microdystrophin gene therapy, including (rAAV) rh74MHCK7.microdystrophin, and further treating the subject with an immunosuppressing regimen, TPE, or both It is recognized that the sera or plasma of a subject, including a human patient, may contain preexisting anti-AAVrh.74 antibodies, and therefore be identifiable as seropositive for AAVrh.74 prior to having received any gene therapy treatment. It is further recognized that a subject, including a human patient, can become seropositive due to receiving an AAV-based gene therapy. Accordingly, the determination can be used to monitor existence and levels of anti-AAVrh.74 antibodies in serum or plasma of such subjects, and further used in determining whether or not an immunosuppressing regimen, TPE, or both should be administered to such subject prior to treatment with rAAV.MHCK7.microdystrophin including AAVrh74.MHCK7.microdystrophin. In this regard, the rAAV.MHCK7.microdystrophin including AAVrh74.MHCK7.microdystrophin can be a first time gene therapy treatment or an additional gene therapy treatment including a redosing treatment. In this regard, the determination of seropositivity in such sera or plasma is used to determine whether the subject is eligible for a first time gene therapy with rAAV.MHCK7.microdystrophin including AAVrh74.MHCK7.microdystrophin; whether the subject is in need of an immunosuppressing regimen and/or TPE, and whether treatment with an immunosuppressing regimen has resulted in clearing the subject sera or plasma from anti-AAVrh74 antibodies to a sufficient level to make the subject eligible for treatment or re-treatment with rAAV.MHCK7.microdystrophin including AAVrh74.MHCK7.microdystrophin. Methods and compositions useful for such determination of the presence of anti-AAVrh.74 antibodies in serum or plasma of a subject include those described in Griffin et al., Adeno-associated Virus Serotype rh74 Prevalence in Muscular Dystrophy Population, American Society of Gene and Cell Therapy, 22^{nd} annual meeting, 2019; and in the International Patent Application PCT/US2021/037314, filed Jun. 15, 2021, corresponding to U.S. Patent Application No. 63/038,957, the entire contents of which are hereby incorporated herein by reference.

An example of antibodies that are utilized in the determination of the presence of anti-AAVrh.74 antibodies in serum or plasma of a subject include the following or as otherwise described herein:

| Antibody | Sequence |
|---|---|
| A | VH: |
| | MDWLWNLLFLMAAAQSAQTQIQLVQSGPELRKPGETVKISCKASGYSFTNY |
| | GMNWVKQTPGKDLKWMGWINTYTGEPTYADDFKGRFAFSLEASANTAYLQI |
| | NDLKNEDMATYFCARGVAHYSDSRFAFDYWGQGTTLTVPS |
| | (SEQ ID NO: 10) |
| | VL: |
| | MHFQVQIFSFLLISASVIMSRGQIVLTQSPAIMSASPGEKVTITCSVSSSV |
| | SYMHWFQQKPGTSPKLWIYYTSNLASGVPGRFSGSGSGTSYSLTISRMEAE |
| | DAATYYCQQRSSYPFTFGSGTKLEIK |
| | (SEQ ID NO: 11) |
| B | VH: |
| | MDWLWNLLFLMAAAQSAQTQIQLVQSGPELKKPGETVKISCKAAGYTFTDY |
| | GMNWVKQAPGEGLKWMGWINTNTGEPTYGDDFKGRFAFSLEASASTAHLQI |
| | NNLKNDDMAIYFCARGNAHPGGSAFVYWGQGTLVTVSA |
| | (SEQ ID NO: 12) |
| | VL: |
| | MHFQVQIFSFLLISASVIMSRGQIVLTQSPAIMSASPGESVTITCSASSGV |
| | TYMHWFQQKPGTSPKNWIYRTSNLASGVPARFSGSGSGTSYSLTISRMEAE |
| | DAATYYCQQRSSYPFTFGSGTKLEIK |
| | (SEQ ID NO: 13) |
| C | VH: |
| | QVKLEESGPELKKPGETVKISCKASGYTFTNYGMNWVKQAPGKGLKWMGWI |
| | NTYTGEPTYADDFKGRFAFSLETSARKVYLQINNLKNEDMATYFCARGSYY |
| | YDSSPAWFAYWGQGTLVTVSA |
| | (SEQ ID NO: 14) |
| | VL: |
| | QIVLTQSPAIMSASPGEKVTITCSASSSVSYMHWFQQKPGTSPKLWIYSTS |
| | NLASGVPARFSGSGSGTSYSLTISRMEAEDAATYYCQQRSTYPFTFGSGTK |
| | LEIKR |
| | (SEQ ID NO: 15) |
| D | VH: |
| | QVKLQESGPELKKPGETVKISCKASGYTFTKYGMNWVKQAPGEGLKWMGWI |
| | NTYTGEPTYADDFKGRFAFSLKTSASTAYLQINNLKNEGTTTYFCARGVDS |
| | SGYGAFAYWGQGTLVTVSA |
| | (SEQ ID NO: 16) |
| | VL: |
| | QIVLTQSPAIMSASPGEKVTITCSASSSVSYMHWFQQKPGTSPKLWIYSTS |
| | NLASGVPARFSGSGSGTSYSLTISRMEAEDAATYYCQQRSFYPFTFGSGTK |
| | LEIKR |
| | (SEQ ID NO: 17) |
| E | VH: |
| | EVQLQESGSDLKKPGETVKISCKASGYTFTNYGMNWVKQAPGKGLKWMGWI |
| | NTYTGEPTYADDFKGRFAFSLETSASTAFLQINNLKYEDTGTYFCTRGTST |
| | MISTFAFVYWGQGTLVTVSA |
| | (SEQ ID NO: 18) |
| | VL: |
| | QIVLTQSPAIMSASPGEKVTITCSASSSVRYMHWFQQKPGTSPKVWIYSTS |
| | NLASGVPARFSGSGSGTSYSLTISRMEAEDAATYYCQQRTYYPFTFGSGTK |
| | LEIKR |
| | (SEQ ID NO: 19) |

Additional examples of antibodies that are utilized in the determination of the presence of anti-AAVrh.74 antibodies in serum or plasma of a subject include monoclonal antibodies comprising: a VH CDR1 amino acid sequence selected from the group consisting of NYGMN (SEQ ID NO: 20), DYGMN (SEQ ID NO: 22), YTFTNYGMN (SEQ ID NO: 20), YTFTKYGMN (SEQ ID NO: 23), and YTFTNYGMN (SEQ ID NO: 21), and/or a VH CDR2 amino acid sequence selected from the group consisting of WINTYTGEPTYADDFKG (SEQ ID NO: 24), WINTNTGEPTYGDDFKG (SEQ ID NO: 25), and WMGWINTYTGEPTY (SEQ ID NO: 26), and/or a VH CDR3 amino acid sequence selected from the group consisting of GVAHYSDSRFAFDY (SEQ ID NO: 27), GNAHPGGSAFVY (SEQ ID NO: 28), RGSYYYDSSPAWFAY (SEQ ID NO: 29), RGVDSSGYGAFAY (SEQ ID NO: 30), and TRGTSTMISTFAFVY (SEQ ID NO: 31), and/or a VL CDR1 amino acid sequence selected from the group consisting of SVSSSVSYIVIH (SEQ ID NO: 32), SASSGVTYMH (SEQ ID NO: 33), SSVSYIVIH (SEQ ID NO: 34), SSVSYIVIH (SEQ ID NO: 34), and SSVRYIVIH (SEQ ID NO: 35), and/or a VL CDR2 amino acid sequence selected from the group consisting of YTSNLAS (SEQ ID NO: 36), RTSNLAS (SEQ ID NO: 37), LWIYSTSNLAS (SEQ ID NO: 38), and VWIYSTSNLAS (SEQ ID NO: 39), and/or a VH CDR3 amino acid sequence selected from the group consisting of QQRSSYPFT (SEQ ID NO: 40), QQRSTYPF (SEQ ID NO: 41), QQRSFYPF (SEQ ID NO: 42), and QQRTYYPF (SEQ ID NO: 43).

Treatment of Muscular Dystrophy, Including Duchenne Muscular Dystrophy (“DMD”) or Becker's Muscular Dystrophy (“BMD”) with a rAAV Comprising Microdystrophin

The invention encompasses a method of treating muscular dystrophy in a human subject in need thereof comprising the step of administering a recombinant adeno-virus associated (rAAV) comprising a heterologous nucleotide sequence encoding microdystrophin and further comprising administering a immunosuppressing regimen. In various embodiments of the invention, the methods comprise administering a recombinant adeno-virus associated (rAAV) comprising a heterologous nucleotide sequence encoding microdystrophin and further comprise administering an anti-inflammatory steroid, including for example prednisone.

In all embodiments of the invention directed to treating a muscular dystrophy with an rAAV comprising microdystrophin and further comprising administering an immuno-suppressing regimen or an anti-inflammatory steroid, the rAAV or microdystrophin encoding nucleotide sequences that can be utilized in the methods of the invention includes those described in WO-2020/123645, WO-2019/209777, WO-2019/195362, WO-2016/115543, WO-2019118806, WO-2017/221145, the contents of each of which is hereby incorporated herein by reference, and include SGT-001, zildistrogene varoparvovec, and PF-06939926.

### Treatment of Limb-Girdle Muscular Dystrophies

Treatment of a Limb-Girdle Muscular Dystrophy (“LGMD”) is an aspect of the invention. It is recognized that the methods of the invention described herein can be utilized in the treatment of Limb-Girdle muscular dystrophies by employing rAAV vectors that are useful for treating such dystrophies. Such rAAV vectors include those described in PCT/US2019/039893 (WO 2020/06458) including AAVrh.74.tMCK.CAPN3; U.S. Patent Application 63/024,338 including rAAVrh.74.MHCK7.DYSF.DV; PCT/US2019/015779 (WO 2019/15474) including scAAVrh.74.MHCK7.hSGCG; PCT/US2020/47339 including AAVrh74.tMCK.hSCGA; PCT/US2020/019892 (WO2020/176614) including scAAVrh74.MHCK7.HSGCB; PCT/US2016/061703 (WO2017/083776) including rAAVrh.74.MHCK7.huAN05, the contents of each of which is hereby incorporated herein by reference.

### Combination Therapies for Treating Muscular Dystrophy

The disclosure provides for combination therapies for treating muscular dystrophy in a human subject in need, including for treating DMD, Becker's muscular dystrophy and Limb Girdle muscular dystrophy, wherein the combination therapy comprises a rAAV and an anti-inflammatory steroid. The disclosure also provides for use of a combination therapy comprising rAAV and an anti-inflammatory steroid for the preparation of a medicament for treating muscular dystrophy, including for treating DMD, Becker's muscular dystrophy and Limb Girdle muscular dystrophy.

The disclosure provides for combination therapies and medicaments comprising the rAAV rAAV.MHCK7.microdystrophin and an anti-inflammatory steroid administered in combination including administered simultaneously, sequentially or at differing time points. The disclosure also provides for combination therapies and medicaments comprising a rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05 and an anti-inflammatory steroid administer in combination including administered simultaneously, sequentially or at differing time points. For example, the anti-inflammatory steroid is a glucocorticoid. In some embodiments, the anti-inflammatory steroid is prednisone, prednisolone, betamethasone, dexamethasone, hydrocortisone, methylprednisolone or deflazacort. In some embodiments, the anti-inflammatory steroid is formulated for oral administration. For the combination therapies and medicaments, the anti-inflammatory steroid may be administered both prior to and after administration of the rAAV. Alternatively, the anti-inflammatory steroid is administered only prior to or only after administration of the rAAV.

In some combination therapies and medicaments, the anti-inflammatory steroid is administered about 12 hour prior to administration of the rAAV or about 24 hours prior to administration of the rAAV or about 36 hours prior to administration of the rAAV or about 48 hours prior to administration of the rAAV or about 60 hours prior to administration of the rAAV or about 72 hours prior to administration of the rAAV or about 96 hours prior to administration. In some combination therapies and medicaments, the inflammatory steroid is administered about 5 days hours prior to administration of the rAAV, about 6 days hours prior to administration of the rAAV, about 7 days hours prior to administration of the rAAV, or about 8 days prior to administration of the rAAV, or about 9 days prior to administration of the rAAV, or about 10 days prior to administration of the rAAV, or about 11 days prior to administration of the rAAV, or about 12 days prior to administration of the rAAV, or about 13 days prior to administration of the rAAV, or about 14 days prior to administration of the rAAV, or about 30 days prior to administration of the rAAV.

In some combination therapies and medicaments, the anti-inflammatory steroid is administered at least once a day for about 7 days prior to administration of the rAAV, or administered at least once a day for about 14 days prior to administration of the rAAV, or administered at least once a day for 21 days, or administered at least once a day for about 28 days prior to administration of the rAAV, or administered at least once a day for about 30 days prior to administration of the rAAV, or administered at least once a day for about 45 days prior to administration of the rAAV, or administered at least once a day for about 60 days prior to administration of the rAAV. In some compositions and medicaments, the anti-inflammatory steroid is administered 30 to 60 days prior to administration of the rAAV.

In exemplary combination therapies and medicaments, the anti-inflammatory steroid is administered prior to administration of the rAAV and the anti-inflammatory steroid is administered at least once a day from day 1 to 30 days after administration of the rAAV or at least once a day from 1 to 60 days after administration of the rAAV or at least once a day from 1 to 7 days after administration of the rAAV or at least once a day from 1 to 14 days after administration of the rAAV or at least once a day from 1 to 21 days after administration of the rAAV, or at least once a day from 1 to 24 days after administration of the rAAV, or at least once a day from 1 to 28 days after administration of the rAAV, or at least from 1 to 30 days after administration of the rAAV, or at least 30 to 60 days after administration of the rAAV.

In any of the combination therapies and medicaments disclosed herein, the combination therapies or medicament may also may comprise an anti-CD20 specific antibody, which is administered in combination with the rAAV and the anti-inflammatory steroid. The anti-CD20 specific antibody is administered prior to administration of the rAAV. In some compositions, the anti-CD20 specific antibody is administered at least 7 days prior to administration of the rAAV. Exemplary anti-CD20 antibodies include rituximab, ocrelizumab or ofatumumab.

In some combination therapies and medicaments, the anti-CD20 specific antibody is administered about 60 days prior to administration of the rAAV, or about 45 days prior to administration the rAAV, or about 30 days prior to administration of the rAAV, about 14 days prior to administration of the rAAV, about 7 days prior to administration of the rAAV and within about 24 hours of the administration of the rAAV. In some compositions, the anti-CD20 antibody is administered 30 to 60 days prior to administration of the rAAV. In some combination therapies and medicaments, the anti-CD20 specific antibody is administered after administration of the rAAV. For example, the anti-CD20 specific antibody is administered both prior to and after administration of the rAAV. Alternatively, the anti-CD20 specific antibody is administered prior to administration of the rAAV or the anti-CD20 specific antibody is administered after administration of the rAAV.

In addition, any of the disclosed combination therapies and medicaments, an immunosuppressing macrolide is administered to the subject in combination with the rAAV and the anti-inflammatory steroid, and optionally the anti-CD-20 antibody. Examples of immunosuppressing macrolides include tacrolimus, pimecrolimus, and sirolimus. In some combination therapies and medicaments, the immunosuppressing macrolide is formulated for oral administration. In some combination therapies and medicaments, the immunosuppressing macrolide may be administered both prior to administration of the rAAV and after administration of the rAAV. Alternatively, the immunosuppressing macrolide is administered prior to administration or the rAAV or the immunosuppressing macrolide is administered after administration of the rAAV.

In some combination therapies and medicaments, the immunosuppressing macrolide is administered at least once a day for at least three days prior to administration of the rAAV, or administered at least 4 days prior to administration of the rAAV, or administered at least 5 days prior to administration of the rAAV, or administered at least 6 days prior to administration of the rAAV, administered at least 7 days prior to administration of the rAAV, or administered at least 10 days prior to administration of the rAAV, or administered at least 14 days prior to administration, or administered at least 30 days prior to administration of the rAAV, or administered at least 45 days prior to administration of the rAAV, or administered at least 60 days prior to administration of the rAAV. In some combination therapies and medicaments, the immunosuppressing macrolide is administered 30 to 60 days prior to administration of the rAAV.

The disclosure also provides for combination therapies for treating muscular dystrophy in a human subject in need thereof comprising co-administering a rAAV and an immunosuppressing regimen, including treating DMD, Becker's muscular dystrophy and Limb Girdle muscular dystrophy, wherein the rAAV and one or more components of the immunosuppressing regime are administered simultaneously, sequentially or at differing time points. In addition, the disclosure also provides for use of a combination therapy comprising a rAAV and an immunosuppressing regimen for the preparation of a medicament for treating muscular dystrophy in a human subject in need thereof, including treating DMD, Becker's muscular dystrophy and Limb Girdle muscular dystrophy, wherein the rAAV and one or more components of the immunosuppressing regimen are administered simultaneously, sequentially or at differing time points. For example, the disclosure provides for combination therapies for treating muscular dystrophy comprising rAAV.MHCK7.microdystrophin and an immunosuppressing regimen, wherein the immunosuppressing regimen comprises one or more of an anti-inflammatory steroid, an anti-CD20 antibody, and an immunosuppressing macrolide, wherein the rAAV and one or more of the components of the immunosuppressing regimen are administered simultaneously, sequentially or at differing time points. For example, the disclosure provides for combination therapies for treating muscular dystrophy comprising a rAAV selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05 and an immunosuppressing regimen, wherein the immunosuppressing regimen comprises one or more of an anti-inflammatory steroid, an anti-CD20 antibody, and an immunosuppressing macrolide, wherein the rAAV and one or more of the components of the immunosuppressing regimen are administered simultaneously, sequentially or at differing time points.

For example, the disclosure provides for use of a combination therapy comprising a rAAV.MHCK7.microdystrophin and an immunosuppressing regimen for the preparation of a medicament for treating muscular dystrophy, wherein the immunosuppressing regimen comprises one or more of an anti-inflammatory steroid, an anti-CD20 antibody, and an immunosuppressing macrolide, wherein the rAAV and one or more of the components of the immunosuppressing regimen are administered simultaneously, sequentially or at differing time points. For example, the disclosure provides for use of a rAAV and am immunosuppressing regimen for treating muscular dystrophy, wherein the rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05, and wherein the immunosuppressing regimen comprises one or more of an anti-inflammatory steroid, an anti-CD20 antibody, and an immunosuppressing macrolide wherein the rAAV and one or more of the components of the immunosuppressing regimen are administered simultaneously, sequentially or at differing time points.

The term immunosuppressing regimen refers to a method of treatment which suppresses or modulates the immune system of the subject. The regimen comprises administration of one or more immune suppressing agents. In some embodiments, the immunosuppressing regimen comprises administering an anti-inflammatory steroid, an anti-CD20 antibody, and an immunosuppressing macrolide.

In an exemplary combination therapy or medicament, the immunosuppressing regimen comprises an anti-inflammatory steroid administered about 24 hours prior to administration of the rAAV. In another exemplary combination therapies or medicaments, the immunosuppressing regimen comprises an anti-inflammatory steroid administered prior to administration of the rAAV and the anti-inflammatory steroid is administered at least once a day from day 1 to 30 days after administration of the rAAV or the anti-inflammatory steroid is administered at least once a day from day 1 to 60 days after administration of the rAAV. In another embodiment, a glucocorticoid anti-inflammatory steroid such as prednisone is administered for at least 60 days following the administration of rAAV at 1 mg/kg.

In any of the disclosed combination therapies and medicaments, in the immunosuppressing regimen, the anti-inflammatory steroid is a glucocorticoid such as prednisone, prednisolone, betamethasone, dexamethasone, hydrocortisone, methylprednisolone or deflazacort. In some combination therapies, the anti-inflammatory steroid is formulated for oral administration.

In additional exemplary combination therapies and medicaments, the immunosuppressing regimens comprises an anti-CD20 specific antibody administered prior to administration of the rAAV. For example, the anti-CD20 antibody is formulated for administration by intravascular infusion. Exemplary anti-CD20 specific antibody include rituximab, ocrelizumab or ofatumumab.

In some combination therapies and medicaments, the anti-CD20 specific antibody is administered at least 14 days prior to administration of the rAAV. In another embodiment, the anti-CD20 specific antibody is administered about 60 days prior to administration of the rAAV, about 45 days prior to administration of the rAAV, about 30 days prior to administration of the rAAV, 14 days prior to administration of the rAAV, about 7 days prior to administration of the rAAV and within about 24 hours of the administration of the rAAV. In addition, the anti-CD20 specific antibody administered for 30 to 60 days prior to administration of the rAAV. The disclosed immunosuppressing regimens also include administering an anti-CD20 specific antibody after administration of the rAAV.

In addition, the disclosed immunosuppressing regimens comprise an immunosuppressing macrolide administered at least once a day for at least three days prior to administration of the rAAV. The immunosuppressing regimens also may comprise an immunosuppressing macrolide administered after administration of the rAAV. In any of the disclosed immunosuppressing regimen, the immunosuppressing macrolide is formulated for oral administration. Exemplary immunosuppressing macrolides include tacrolimus, pinecrolimus or sirolimus.

In some embodiments, the disclosed immunosuppressing regimen is administered from 30 to 60 days prior to administration of the rAAV. In addition, the immunosuppressing regimen is administered about 60 days prior to administration of the rAAV, about 45 days prior to administration of the rAAV, about 30 days prior to administration the rAAV, about 14 days prior to administration of the rAAV, about 7 days prior to administration of the rAAV, about 24 hours, or about 12 hours prior to administration of the rAAV.

In a particular embodiment, the disclosure provides for combination therapies for treating muscular dystrophy in a human subject in need thereof, wherein the combination therapies comprises a rAAV and an immunosuppressing regimen, wherein the immunosuppressing regimen comprises i) an anti-inflammatory steroid orally administered about 24 hours prior to administration of the rAAV, and an anti-inflammatory steroid administered at least once a day from day 1 to 30 days after administration of the rAAV or an the anti-inflammatory steroid administered at least once a day from day 1 to 60 days after administration of the rAAV, ii) an anti-CD20 antibody intravenously administered about 14 days prior to administration of the rAAV, about 7 days prior to administration of the rAAV and within about 24 hours of the administration of the rAAV, and optionally comprising an anti-CD20 antibody administered after administration of the rAAV, iii) an immunosuppressing macrolide orally administered at least once a day for at least three days prior to administration of the rAAV, and optionally comprising an immunosuppressing macrolide administered after administration of the rAAV. For example, the anti-inflammatory steroid is prednisone, prednisolone, betamethasone, dexamethasone, hydrocortisone, methylprednisolone or deflazacort, the anti-CD20 specific antibody is rituximab, ocrelizumab or ofatumumabone or more of an anti-inflammatory steroid, an anti-CD20 antibody, and an immunosuppressing macrolide, the immunosuppressing macrolide is tacrolimus, pinecrolimus or sirolimus. In an exemplary embodiment, the immunosuppressing regimen comprises the anti-inflammatory steroid prednisone or prednisolone, the anti-CD20 antibody rituximab, and the immunosuppressing macrolide sirolimus.

In a particular embodiment, the disclosure provides for use of combination therapy comprising a rAAV and an immunosuppressing regimen for treating Limb Girdle muscular dystrophy in a human subject in need thereof, wherein the in rAAV is selected from the group consisting of: AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05, and wherein the combination therapy comprises a rAAV and an immunosuppressing regimen, wherein the immunosuppressing regimen comprises i) an anti-inflammatory steroid orally administered about 24 hours prior to administration of the rAAV, and an anti-inflammatory steroid administered at least once a day from day 1 to 30 days after administration of the rAAV or an the anti-inflammatory steroid administered at least once a day from day 1 to 60 days after administration of the rAAV, ii) an anti-CD20 antibody intravenously administered about 14 days prior to administration of the rAAV, about 7 days prior to administration of the rAAV and within about 24 hours of the administration of the rAAV, and optionally comprising an anti-CD20 antibody administered after administration of the rAAV, iii) an immunosuppressing macrolide orally administered at least once a day for at least three days prior to administration of the rAAV, and optionally comprising an immunosuppressing macrolide administered after administration of the rAAV. For example, the anti-inflammatory steroid is prednisone, prednisolone, betamethasone, dexamethasone, hydrocortisone, methylprednisolone or deflazacort, the anti-CD20 specific antibody is rituximab, ocrelizumab or ofatumumabone or more of an anti-inflammatory steroid, an anti-CD20 antibody, and an immunosuppressing macrolide, the immunosuppressing macrolide is tacrolimus, pinecrolimus or sirolimus. In an exemplary embodiments, the immunosuppressing regimen comprises the anti-inflammatory steroid prednisone or prednisolone, the anti-CD20 antibody rituximab, and the immunosuppressing macrolide sirolimus.

The disclosure also provides combination therapies for treating muscular dystrophy in a human subject in need wherein the combination therapy comprises a dose of rAAV, e.g. a second dose, wherein the subject's plasma is subjected to at least one therapeutic plasma exchange (TPE) prior to administration of a second dose of recombinant adeno-virus associated (rAAV) and wherein the subject was administered a first dose of rAAV prior to being subjected to TPE. For example, the rAAV is rAAV.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, or rAAVrh.74.MHCK7.huAN05. The muscular dystrophy is DMD, Becker's muscular dystrophy or Limb Girdle muscular dystrophy.

In addition, the disclosure provides for use of a combination therapy for the preparation of a medicament for treating muscular dystrophy in a human subject in need, the combination therapy comprises a dose of rAAV, e.g. “a second dose of rAAV, administered to the subject, wherein the subject's plasma to at least one therapeutic plasma exchange (TPE) prior to administration of a second dose of recombinant adeno-virus associated (rAAV) and wherein the subject was administered a first dose of rAAV prior to being subjected to TPE. For example, the rAAV is rAAV.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, or rAAVrh.74.MHCK7.huAN05. The muscular dystrophy is DMD, Becker's muscular dystrophy or Limb Girdle muscular dystrophy.

In any of the disclosed combination therapies and uses, the subject's plasmas is subject to at least two TPE or at least three TPE prior to administration of the 2^{nd} dose or rAAV. In some embodiments, the subject's plasma is subject to at least four TPE prior to administration of the 2^{nd} dose of rAAV, or the subject's plasma is subject five TPE prior to administration of the 2^{nd} dose of rAAV, or the subject's plasma is subject six TPE prior to administration of the 2^{nd} dose of rAAV, or the subject's plasma is subject seven TPE prior to administration of the 2^{nd} dose of rAAV.

The disclosure provides for combination therapies for treating muscular dystrophy in a human subject in need thereof, wherein the combination therapies comprises a rAAV administered to the subject and wherein the subject's plasma is subjected to at least one therapeutic plasma exchange (TPE) prior to administering the rAAV and wherein the rAAV rAAV.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, or rAAVrh.74.MHCK7.huAN05. The muscular dystrophy is DMD, Becker's muscular dystrophy or Limb Girdle muscular dystrophy.

The disclosure provides for use of a combination therapy for the preparation of a medicament for treating muscular dystrophy in a human subject in need thereof, wherein the combination therapy comprises a rAAV administered to the subject, and wherein the subject's plasma to at least one therapeutic plasma exchange (TPE) prior to administering the rAAV and wherein the rAAV rAAV.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, or rAAVrh.74.MHCK7.huAN05. The muscular dystrophy is DMD, Becker's muscular dystrophy or Limb Girdle muscular dystrophy.

In any of the disclosed combination therapies and uses, the subject's plasma is subjected to at least two TPE prior to administering the rAAV, at least three TPE prior to administering the rAAV, at least four TPE prior to administering the rAAV, at least five TPE prior to administering the rAAV, at least six TPE prior to administering the rAAV or at least seven TPE prior to administering prior to administering the rAAV. In these disclosed combination therapies and uses, the subject was administered an anti-inflammatory steroid about 24 hours prior to administration of the rAAV. In addition, in some embodiments, the subject is administered an anti-inflammatory steroid at least once a day from day 1 to 60 days after administration of the rAAV. For example, the anti-inflammatory steroid is formulated for oral administration. In addition, the anti-inflammatory steroid is a glucocorticoid such as prednisone, prednisolone, betamethasone, dexamethasone, hydrocortisone, methylprednisolone or deflazacort.

In any of the disclosed combination therapies and uses, the subject's plasma is subjected to TPE for at least 9 days prior to administration of the rAAV, at least 7 days prior to administration, 5 days prior to administration, or 2 days prior to administration. In addition, there is about 24 to about 48 hours between sessions of TPE carried out on the subject's plasma prior to administration of the rAAV. In a particular embodiment, the subject's plasma is subjected to at least two TPE prior to administration of the rAAV, wherein there is about 48 hours between the TPE.

In any of the combination therapies and uses described herein, the subject has a level of anti-AAVrh.74 antibodies of about 1:400 or less at the time of administration of the rAAV. For example, the subject has a level of anti-AAVrh.74 antibodies of about 1:100 to about 1:400 at the time of administration of the rAAV or a level of anti-AAVrh.74 antibodies of about 1:100 to 1:300, or a level of anti-AAVrh.74 antibodies of about 1:100 to 1:200, or a level of anti-AAVrh.74 antibodies of about 1:250 to 1:500, or a level of anti-AAVrh.74 antibodies of about 1:200 to 1:400. The antibody titer is determined as total antibody binding titer. In any of the disclosed combination therapies and uses for treating muscular dystrophy, the presence of anti-AAVrh.74 antibodies was determined in serum or plasma of said subject before administration of rAAV, after administration of rAAV, before an immune response or adverse event is observed or after an immune response or adverse event is observed. In addition, the presence of anti-AAVrh.74 antibodies is determined prior to the step of administering an immunosuppressing regimen or TPE. For example, the presence of anti-AAVrh.74 antibodies is determined prior to any administration of any combination therapies or medicament comprising an AAV to said subject or prior to administration of any combination therapies or medicament comprising AAVrh.74 to said subject.

In addition, in the disclosed combination therapies and uses, the level of anti-AAVrh.74 antibodies in serum or plasma of said subject is used as a positive control. For example, the positive control utilizes an anti-AAVrh.74 monoclonal antibody, such as any of the anti-AAVrh.74 monoclonal antibodies described herein.

In any of the disclosed and uses, the presence of anti-AAVrh.74 antibodies in the subject is determined using an quantitative method, wherein said subject is identified as seropositive for anti-AAVrh.74 antibodies based said quantitation, and wherein said immunosuppressing regimen or TPE is selectively is administered to the seropositive subject.

The disclosure provides for the following additional aspects:

Claim 1. A method of treating muscular dystrophy in a human subject in need thereof comprising the step of administering a recombinant adeno-virus associated (rAAV) and an anti-inflammatory steroid, wherein the rAAV is serotype AAVrh.74 and the rAAV comprises the expression cassette of SEQ ID NO: 9.

Claim 2. The method of claim 1 wherein the rAAV is administered at a dose of 1.33×10¹⁴ vg/kg. The dose of 1.33×10¹⁴ is determined utilizing a linear qPCR DNA standard, corresponding to 2×10¹⁴ as determined by a supercoiled qPCR DNA standard for titer determination.

Claim 3. The method of claim 1 or 2 wherein the subject is suffering from Duchene Muscular Dystrophy, has not previously received a rAAVrh.74-based gene therapy, and has been determined to be seropositive for rAAV.rh74 antibodies.

Claim 4. The method of claim 3 wherein the subject has been determined to be seropositive for rAAVrh.74 antibodies based on an ELISA immunoassay, and wherein the subject exhibits an absorbance ratio of >2.00 at a serum dilution of 1:400.

Claim 5. The method of any one of claims 1-3, further comprising the step of determining the presence of anti-AAVrh.74 antibodies in serum or plasma of said subject prior to any administration of a rAAVrh74 to the subject.

Claim 6. The method of claim 5 wherein the determination of the presence of anti-AAVrh.74 antibodies is determined by an ELISA immunoassay, and wherein the subject exhibits an absorbance ratio of >2.00 at a serum dilution of 1:400.

Claim 7. The method of any one of claims 1-6 wherein the anti-inflammatory steroid is administered orally.

Claim 8. The method of any one of claims 1-7, wherein the anti-inflammatory steroid is administered about 12 hours prior to administration of the rAAV.

Claim 9. The method of any one of claims 1-7, wherein the anti-inflammatory steroid is administered at least 12 hours prior to administration of the rAAV.

Claim 10. The method of any one of claims 1-7, wherein the anti-inflammatory steroid is administered at least 12 hours prior to administration of the rAAV.

Claim 11. The method of any one of claims 1-7, wherein the anti-inflammatory steroid is administered at least 24 hours prior to administration of the rAAV.

Claim 12. The method of any one of claims 1-11, wherein the anti-inflammatory steroid is administered at least once a day from day 1 to about 30 days after administration of the rAAV or at least once a day from 1 to 60 days after administration of the rAAV.

Claim 13. The method of any one of claims 1-11, wherein the anti-inflammatory steroid is administered at least once a day for at least 30 days after the administration of rAAV.

Claim 14. The method of any one of claims 1-11, wherein the anti-inflammatory steroid is administered at least once a day for at least 60 days after the administration of rAAV.

Claim 15. The method of any one of claims 1-14 wherein the anti-inflammatory steroid is glucocorticoid.

Claim 16. The method of claim 13 wherein the glucocorticoid is prednisone.

In addition, the disclosure provides for the following additional aspects:

Claim 1. A method of treating muscular dystrophy in a human subject in need thereof comprising the steps of

a) subjecting the subject's plasma to at least one therapeutic plasma exchange (TPE) prior to administering recombinant adeno-virus associated (rAAV)
b) administering rAAV, wherein the rAAV is serotype rhAAVrh.74 and the rAAV comprises the expression cassette of SEQ ID NO: 9

Claim 2. The method of claim 1 wherein the subject's plasma is subjected to at least two TPE, at least three TPE, at least four TPE, at least five TPE rAAV, at least six TPE or at least seven TPE prior to administering.

Claim 3. The method of claim 1 or 2 wherein the subject's plasma is subjected to TPE for at least 9 days prior to administration of the rAAV, at least 7 days prior to administration, 5 days prior to administration, or 2 days prior to administration.

Claim 4. The method of any one of claims 1-3 wherein the subject plasma is subjected to TPE on the day the rAAV is administered.

Claim 5. The method of any one of claims 1-4, wherein the subject's plasma is subjected to at least two TPE, wherein there is about 48 hours in between the TPE.

Claim 6. The method of any one of claims 1-5 wherein the rAAV is administered at a dose of 1.33×10¹⁴. The dose of 1.33×10¹⁴ is determined utilizing a linear qPCR DNA standard corresponding to 2×10¹⁴ as determined by a supercoiled qPCR DNA standard for titer determination.

Claim 7. The method of any one of claims 1-6 wherein the subject is suffering from Duchene Muscular Dystrophy, and the subject has been determined to be seropositive for rAAVrh.74 antibodies.

Claim 8. The method of claim 7 wherein the subject was administered an AAVrh.74 at least once prior to the administering step of claim 1b.

Claim 9. The method of claim 7 wherein the subject has been determined to be seropositive for rAAVrh7 antibodies based on an ELISA immunoassay, and wherein the subject exhibits an absorbance ratio of >2.00 at a serum dilution of 1:100.

Claim 10. The method of any one of claims 1-9, further comprising the step of determining the presence of anti-AAVrh.74 antibodies in serum or plasma of said subject prior to any administration of the rAAVrh.74.

Claim 11. The method of claim 10 wherein the determination of the presence of anti-AAVrh.74 antibodies is determined by an ELISA immunoassay, and wherein the subject exhibits an absorbance ratio of >2.00 at a serum dilution of 1:100.

Claim 12. The method of any one of claims 1-11, further comprising administering an anti-inflammatory steroid.

Claim 13. The method of claim 12 wherein the anti-inflammatory steroid is administered orally.

Claim 14. The method of claim 12 or 13, wherein the anti-inflammatory steroid is administered about 12 hours prior to administration of the rAAV.

Claim 15. The method of claim 12 or 13, wherein the anti-inflammatory steroid is administered at least 12 hours prior to administration of the rAAV.

Claim 16. The method of claim 12 or 13, wherein the anti-inflammatory steroid is administered at least 12 hours prior to administration of the rAAV.

Claim 17. The method of claim 12 or 13, wherein the anti-inflammatory steroid is administered at least 24 hours prior to administration of the rAAV.

Claim 18. The method of any one of claims 12-17, wherein the anti-inflammatory steroid administered at least once a day from day 1 to about 30 days after administration of the rAAV or at least once a day from 1 to 30 days after administration of the rAAV.

Claim 19. The method of any one of claims 12-17, wherein the anti-inflammatory steroid is administered at least once a day for at least 30 days after the administration of rAAV.

Claim 20. The method of any one of claims 12-17, wherein the anti-inflammatory steroid is administered at least once a day for at least 60 days after the administration of rAAV.

Claim 21. The method of any one of claims 12-17 wherein the anti-inflammatory steroid is glucocorticoid.

Claim 22. The method of claim 21 wherein the glucocorticoid is prednisone.

The following EXAMPLES are provided by way of illustration and not limitation. Described numerical ranges are inclusive of each integer value within each range and inclusive of the lowest and highest stated integer.

### EXAMPLES

### Example 1

A) Generation of the AAVrh74.MHCK7.Micro-Dystrophin Construct

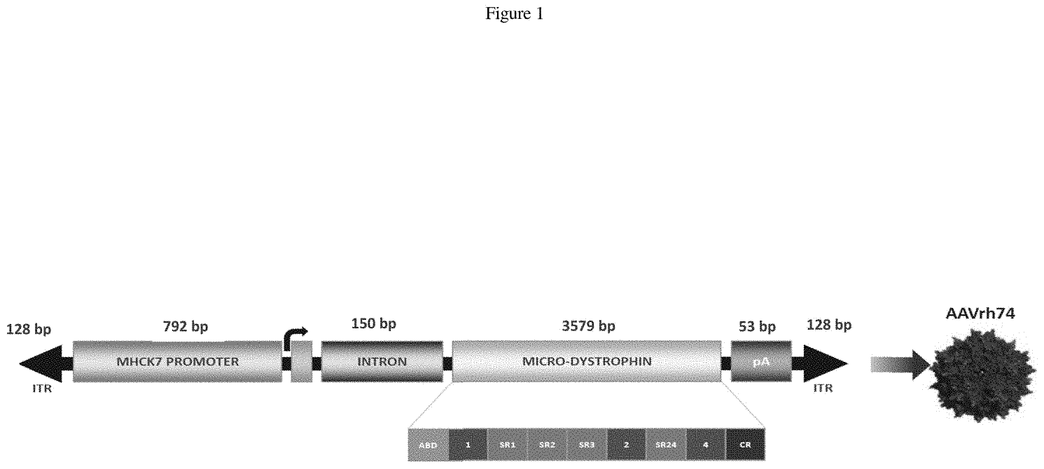

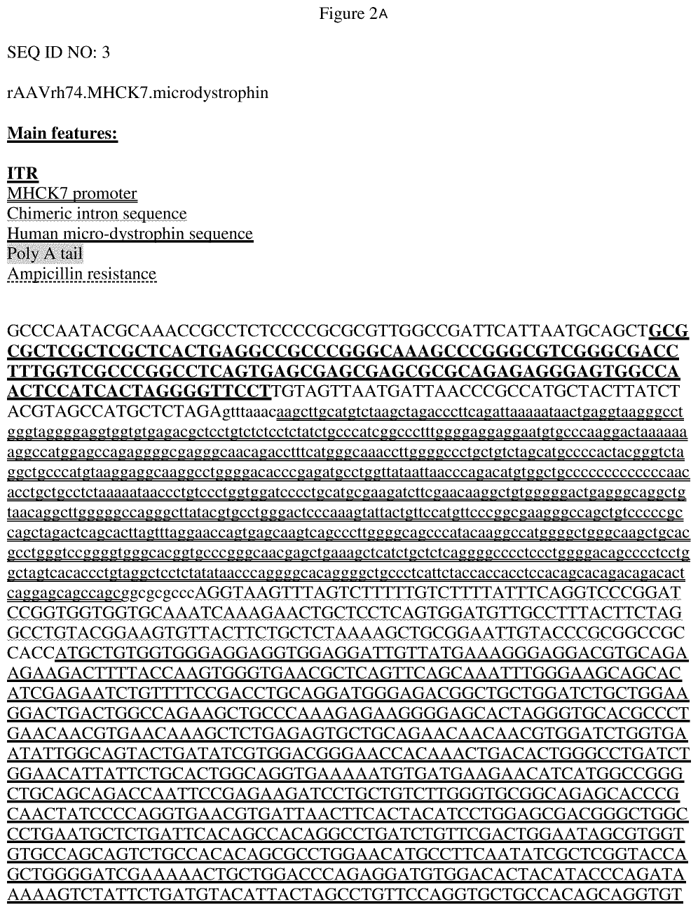

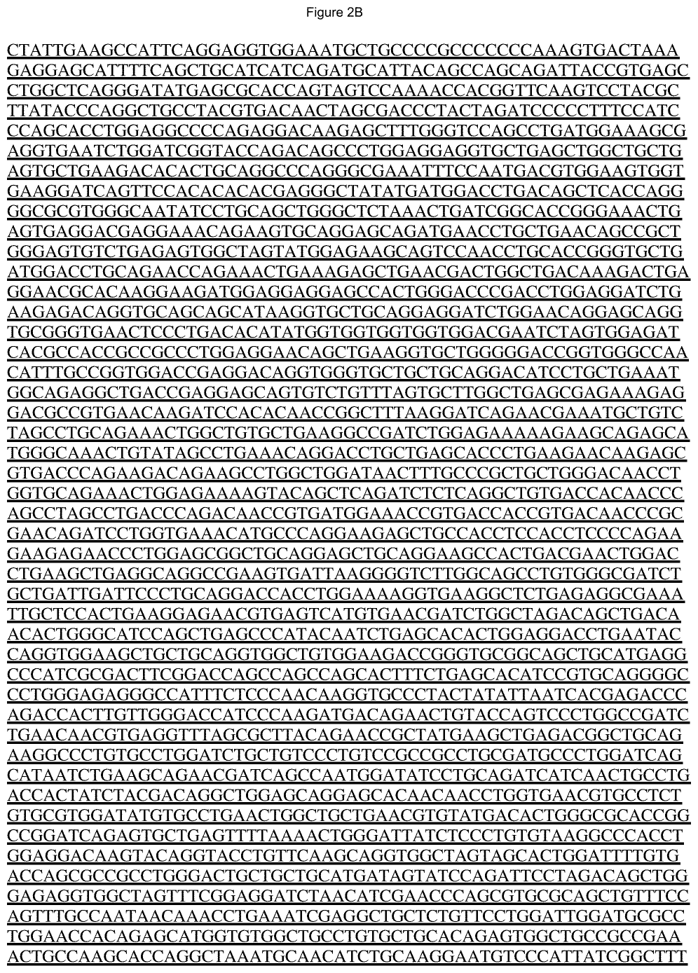

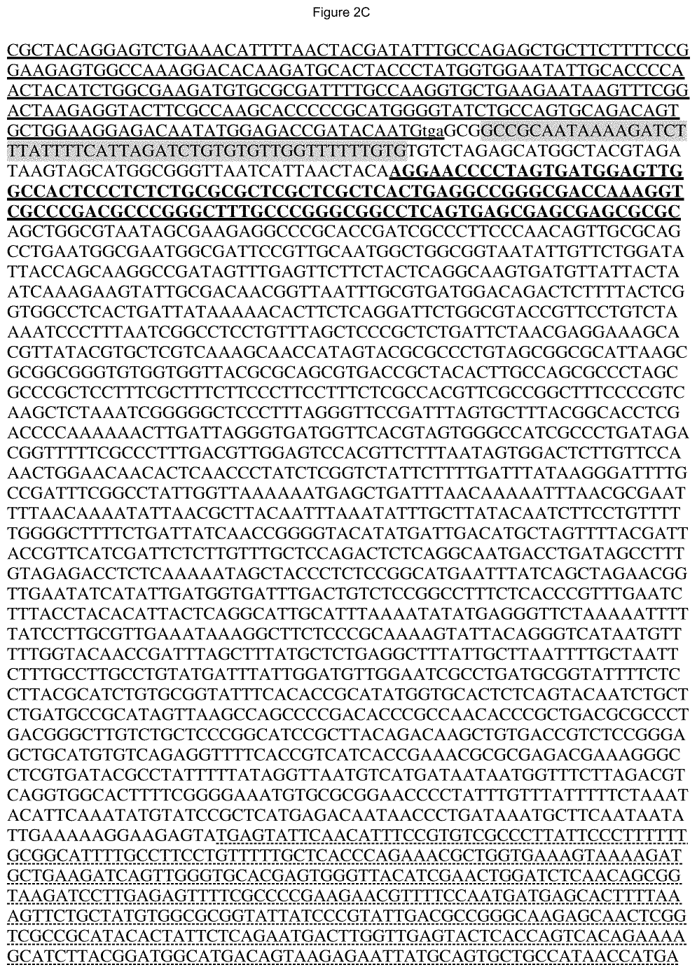

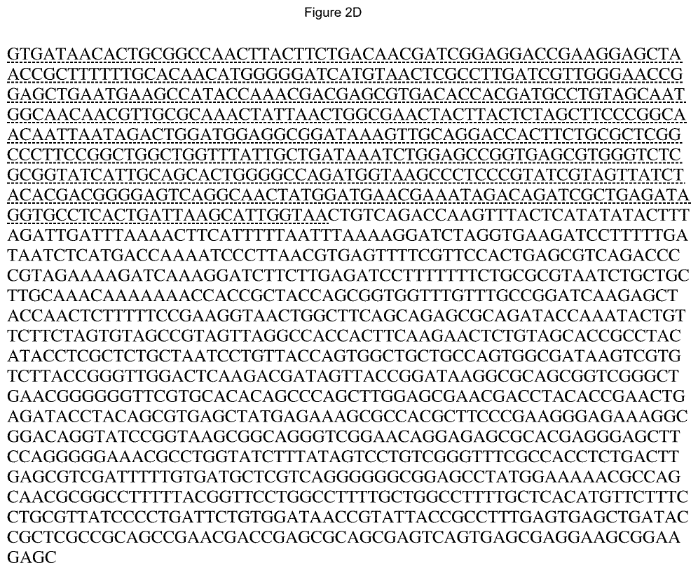

The AAVrh74.MHCK7.micro-dystrophin plasmid contains a human micro-dystrophin cDNA expression cassette flanked by AAV2 inverted terminal repeat sequences (ITR) (see FIG. 1). The micro-dystrophin construct was characterized by an in-frame rod deletion (R4-R23), while hinges 1, 2 and 4 and cysteine rich domain remain producing a 138 kDa protein. The expression of the micro-dystrophin protein (3579 bp) was guided by a MHCK7 promoter (792 bp). The plasmid was constructed from the rAAV.MCK.micro-dystrophin plasmid by removing the MCK promoter and inserting the MHCK7 promoter. After the core promoter, the 53 bp endogenous mouse MCK Exon1 (untranslated) is present for efficient transcription initiation, followed by the SV40 late 16S/19S splice signals (150 bp) and a small 5′UTR (61 bp). The intron and 5′ UTR are derived from plasmid pCMVB (Clontech). The micro-dystrophin cassette had a consensus Kozak immediately in front of the ATG start and a small 53 bp synthetic polyA signal for mRNA termination. The human micro-dystrophin cassette contained the (R4-R23/Δ71-78) domains as previously described by Harper et al. (Nature Medicine 8, 253-261 (2002)). The complementary DNA was codon optimized for human usage and synthesized by GenScript (Piscataway, N.J.) (Mol Ther 18, 109-117 (2010)). The only viral sequences included in this vector were the inverted terminal repeats of AAV2, which are required for both viral DNA replication and packaging. The micro-dystrophin cassette has a small 53 bp synthetic polyA signal for mRNA termination.

Previous studies have validated cardiac expression using MHCK7 promoter (Salva et al. Mol Ther 15, 320-329 (2007) and AAVrh.74 achieving skeletal, diaphragm, and cardiac muscle expression (Sondergaard et al. Annals of clinical and Transl Neurology 2, 256-270 (2015)), The sequence of construct of FIG. 1 was encapsidated into AAVrh.74 virions. The molecular clone of the AAVrh.74 serotype was cloned from a rhesus macaque lymph node and is discussed in in Rodino-Klapac et al. Journal of Translational medicine 5, 45 (2007).

TABLE 1
shows the molecular features of the plasmid AAVrh74.MHCK7.
micro-dystrophin (SEQ ID NO: 3)
Molecular Features of plasmid rAAV.MHCK7.micro-
dystrophin

| TYPE | START | END | NAME | DESCRIPTION |
|---|---|---|---|---|
| REGION | 55 | 182 | 5′ ITR | Wild-type AAV2 inverted |
| | | | | terminal repeat |
| REGION | 244 | 1035 | MHCK7 | Mouse myosin heavy chain |
| | | | | complex - E |
| | | | | box muscle creatine kinase |
| | | | | fusion enhancer/promoter |
| REGION | 1045 | 1194 | Chimeric | 5′ donor site from human |
| | | | intron | β-globin gene |
| | | | | and the branchpoint and |
| | | | | 3′ splice |
| | | | | acceptor site from IgG heavy |
| | | | | chain variable region |
| GENE | 1205 | 4783 | huDys | Human micro-dystrophin |
| | | | cDNA | cDNA |
| REGION | 4786 | 4838 | PolyA | Synthetic PolyA |
| REGION | 4894 | 5021 | 3′ ITR | Wild-type AAV2 inverted |
| | | | | terminal repeat |
| GENE | 6760 | 7619 | AmpR | β-lactamase gene |
| REGION | 7823 | 8442 | On | Plasmid origin of |
| | | | | replication |

B) Generation of the AAVrh74.MHCK7.Micro-Dystrophin Construct from and Plasmid Encoding Kanamycin (Kan) Resistance

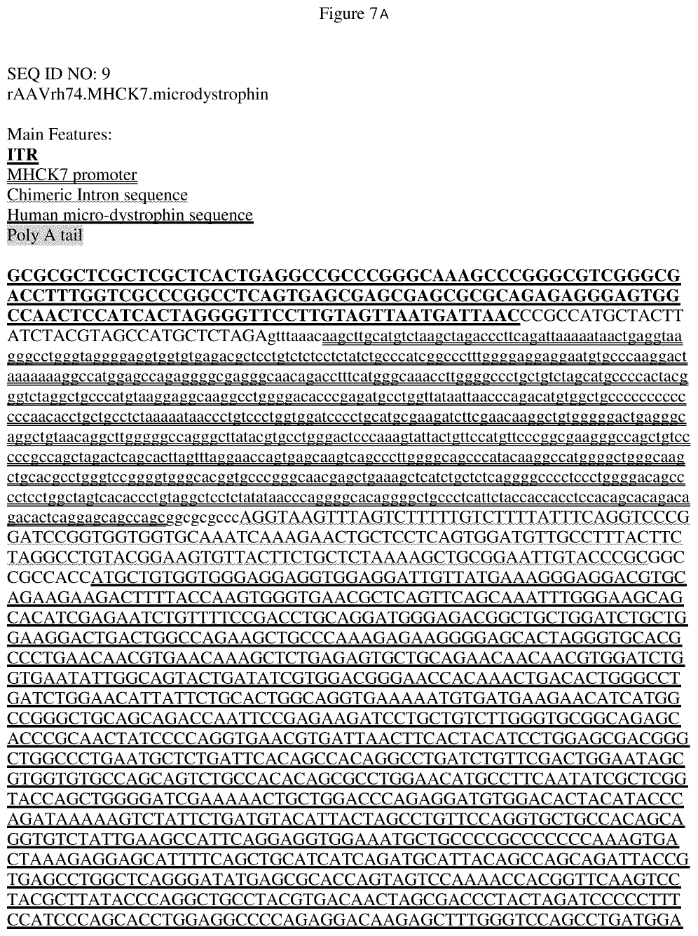

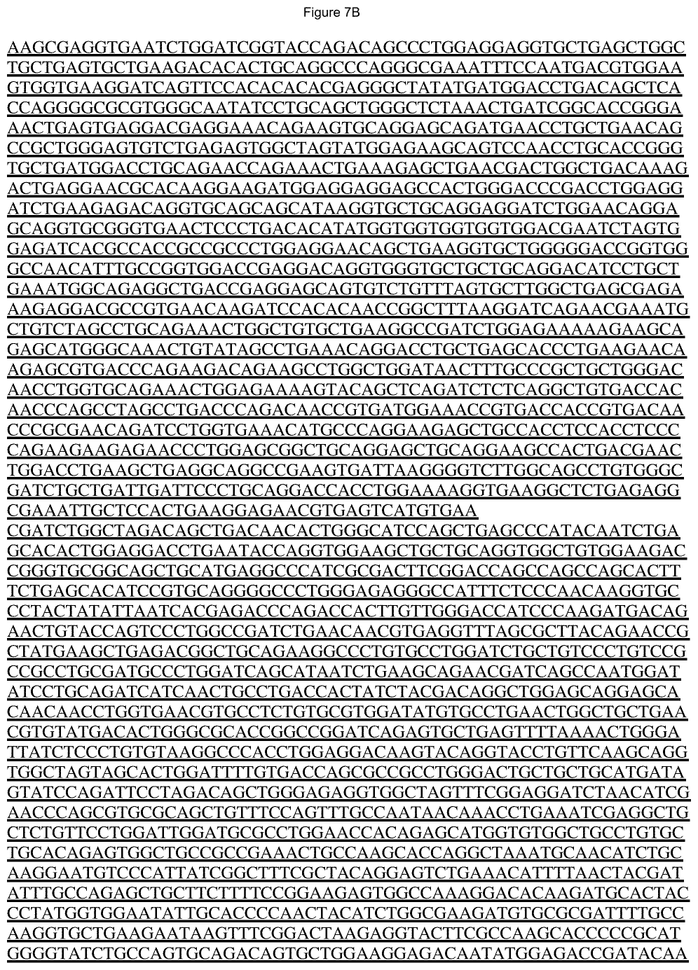

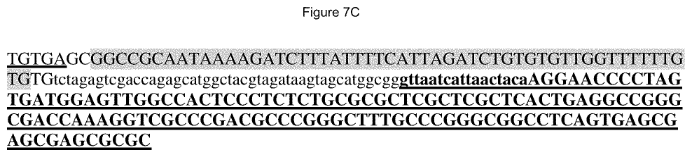

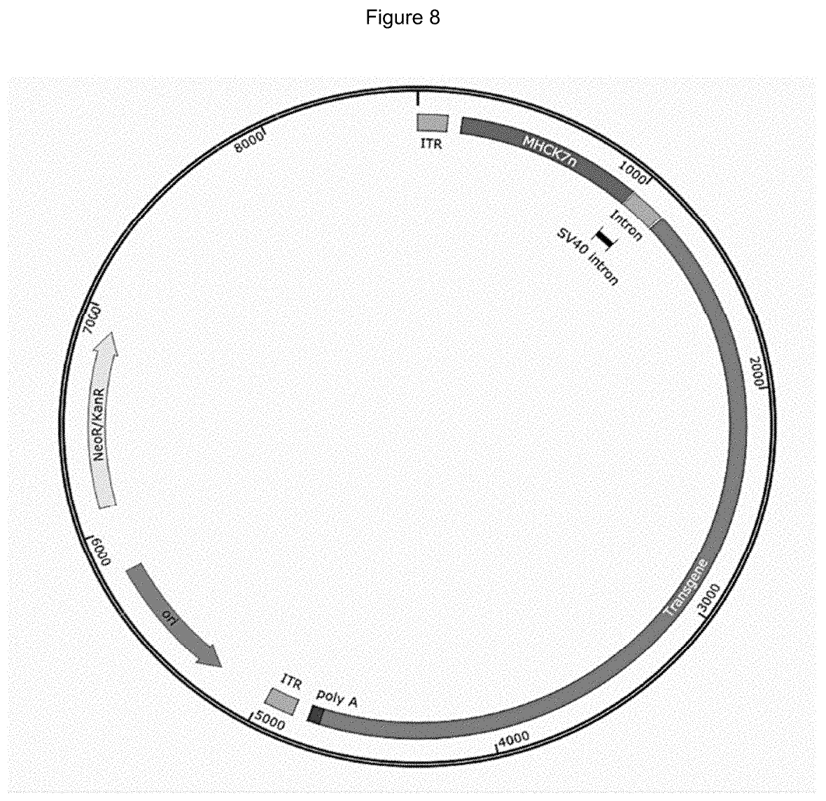

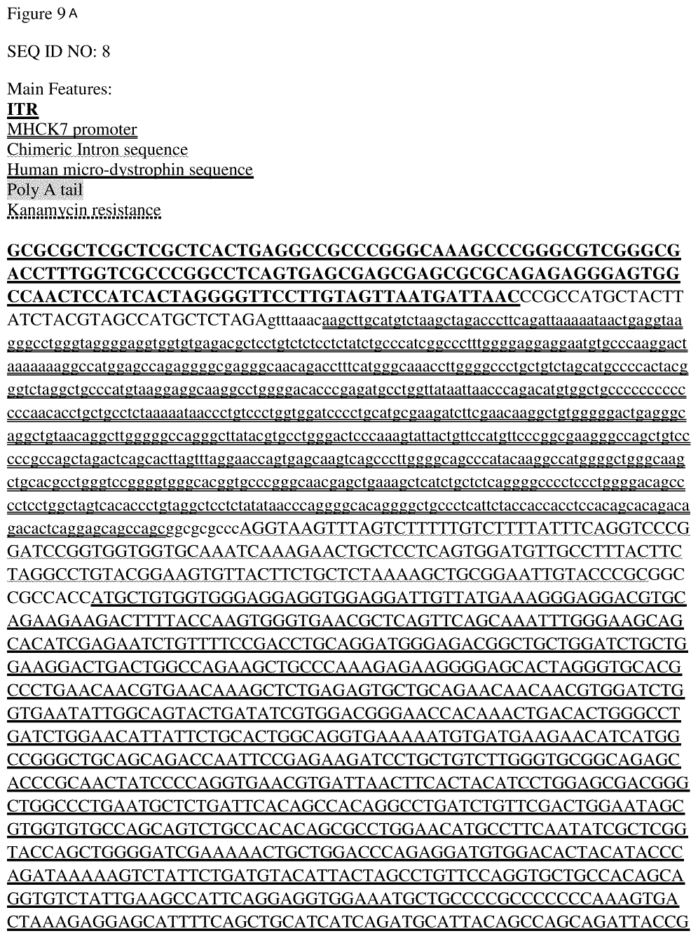

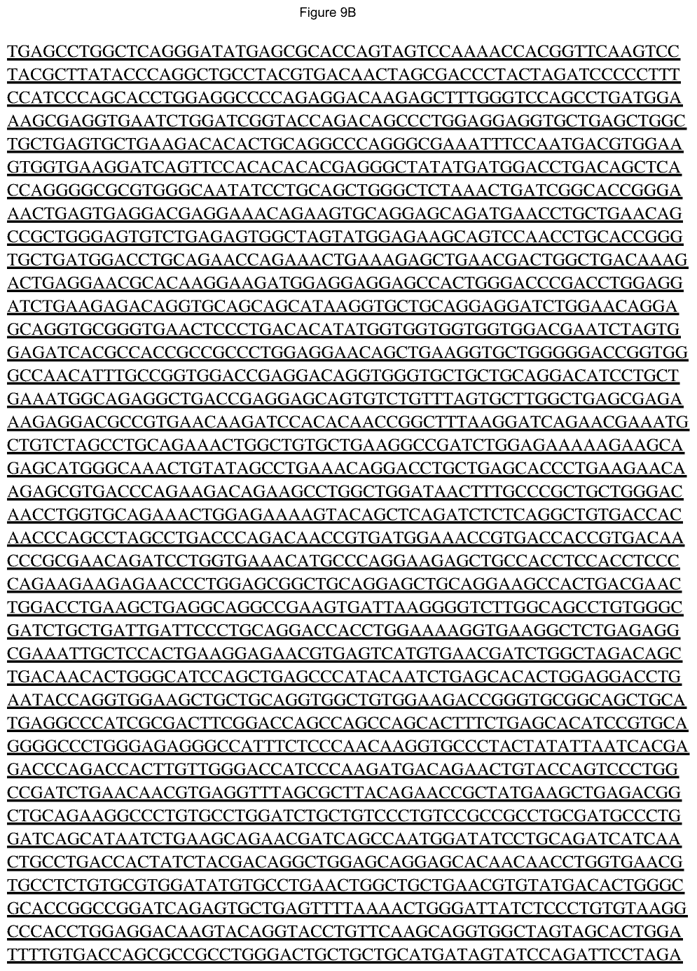

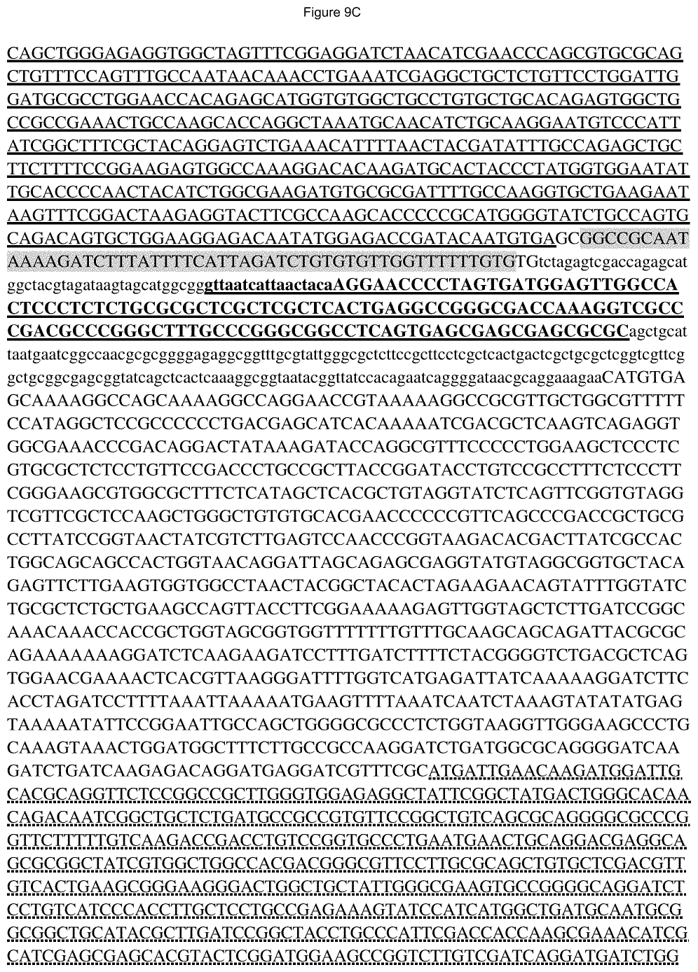

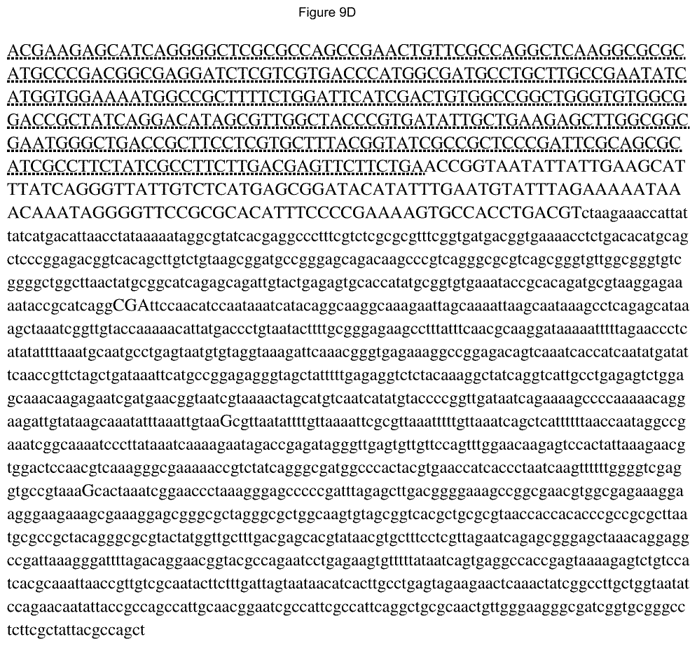

Cloning of MHCK7.μDys.KAN was achieved by isolating the MHCK7.μDys fragment from an MHCK7.μDys.AMP plasmid and the Kanamycin Backbone, and annealing them using the NEBuilder cloning workflow. The MHCK7.μDys fragment was isolated via restriction enzyme digestion with SnaBI. The digestion was performed in a 504, total reaction in 1× CutSmart Buffer (NEB) and 1 μL SnaBI, at 37° C. for 1 hour. The resulting fragment was isolated via electrophoresis using a 1% Agarose gel, running at 105 volts for 1.5 hours. The band corresponding to the MHCK7.μDys insert was cut out and purified using a gel purification kit (Macherey-Nagel). The resulting fragment had a DNA concentration of 10 ng/μL. The Kan backbone fragment was isolated via XbaI restriction enzyme digestion in a 50 μL reaction with 1× CutSmart Buffer (NEB) and 1 μL XbaI, at 37° C. for 1 hour. The resulting fragment was isolated via electrophoresis using a 1% Agarose gel, running at 105 volts for 1.5 hours. The band corresponding to the Kan Backbone was cut out and purified via gel purification kit (Macherey-Nagel). The resulting fragment had a DNA concentration of 8.1 ng/μL. The two fragments were annealed using the NEB Builder cloning workflow, which has the ability to join two fragments with overlapping sequences. The NEBuilder cloning reaction was performed per manufacturer protocol at 50° C. for 15 minutes, using a 1:1 ratio of MHCK7.μDys to Kanamycin backbone in 1× NEBuilder HiFi DNA Assembly Master Mix for a total reaction volume of 20 μL. The resulting clone was transformed into NEB® Stable Competent E. coli (C3040) by adding 2.54, cloning product to the cells followed by 30 minutes on ice, then 30 seconds at 42° C. and an additional 5 minutes on ice. After transformation, 95 μL of outgrowth media was added to the cells and allowed to grow at 30° C. for 1.5 hours, shaking at 225 rpm. Following outgrowth, 450 μL of these cells was plated on a 50 μg/mL kanamycin LB agar plate and incubated overnight at 30° C. in a dry incubator. A colony was picked from this plate and grown up overnight in LB containing 50 μg/mL kanamycin. DNA was isolated from 3 mL of this culture using QIAprep® Spin Miniprep Kit (Qiagen). This DNA was used to confirm the cloning product. The cloning product was confirmed via restriction enzyme digestion with PmeI, MscI, and SmaI followed by gel electrophoresis. The cloning product was additionally confirmed via sequencing. The resultant plasmid is set forth in SEQ ID NO: 8, and shown in FIGS. 8 and 9. The sequence of construct of FIG. 7 which corresponds to that of SEQ ID NO: 9, and nucleotides 1-4977 of SEQ ID NO: 8, was encapsidated into AAVrh.74 virions as described above.

C) Generation of the pAAV.MCK.Micro-Dystrophin Construct

The pAAV.MCK.micro-dystrophin plasmid was constructed by inserting the MCK expression cassette driving a codon optimized human micro-dystrophin cDNA sequence into the AAV cloning vector psub201 (Samulski et al., J. Virol. 61(10):3096-3101). A muscle-specific regulatory element was included in the construct to drive muscle-specific gene expression. This regulatory element comprised the mouse MCK core enhancer (206 bp) fused to the 351 bp MCK core promoter (proximal). After the core promoter, the construct comprises the 53 bp endogenous mouse MCK Exon1 (untranslated) for efficient transcription initiation, followed by the SV40 late 16S/19S splice signals (97 bp) and a small 5′UTR (61 bp). The intron and 5′ UTR was derived from plasmid pCMVB (Clontech). The micro-dystrophin cassette has a consensus Kozak immediately in front of the ATG start and a small 53 bp synthetic polyA signal for mRNA termination. The human micro-dystrophin cassette contains the (R4-R23/Δ71-78) domains as previously described by Harper et al. Nat. Med. 8(3):253-61, 2002

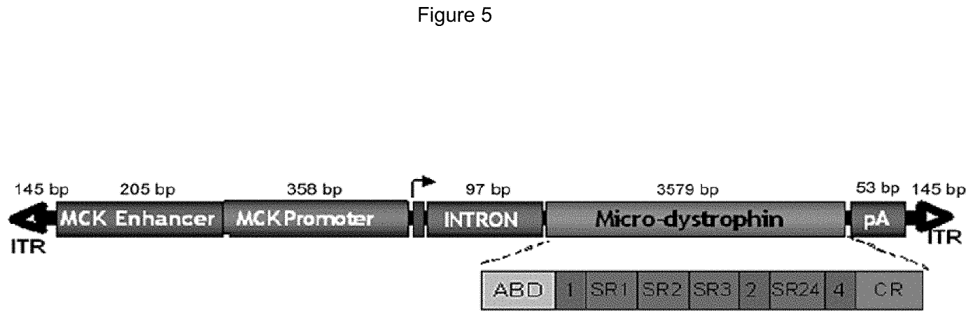

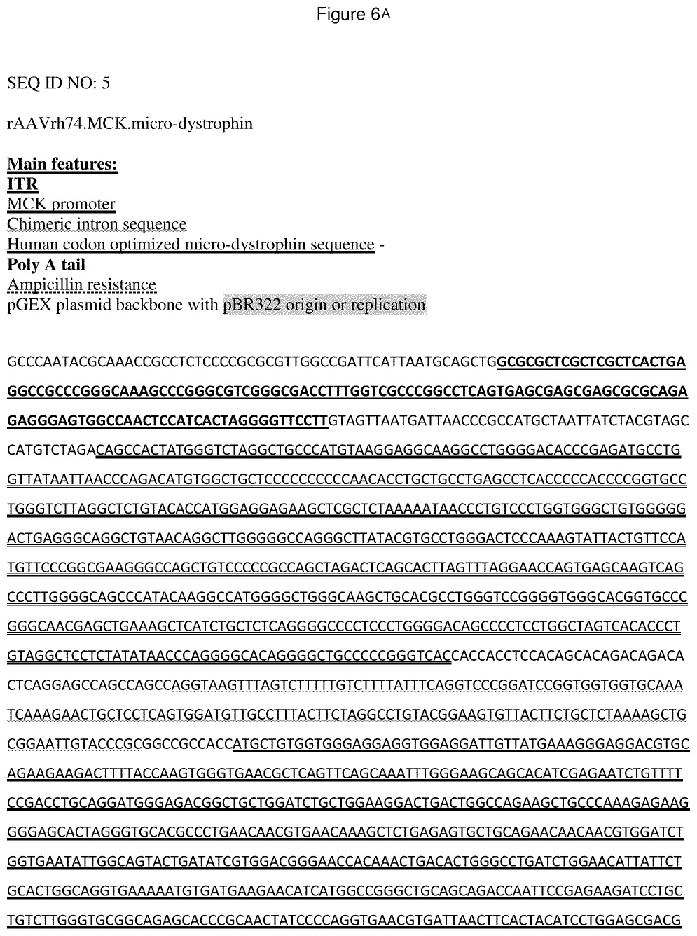

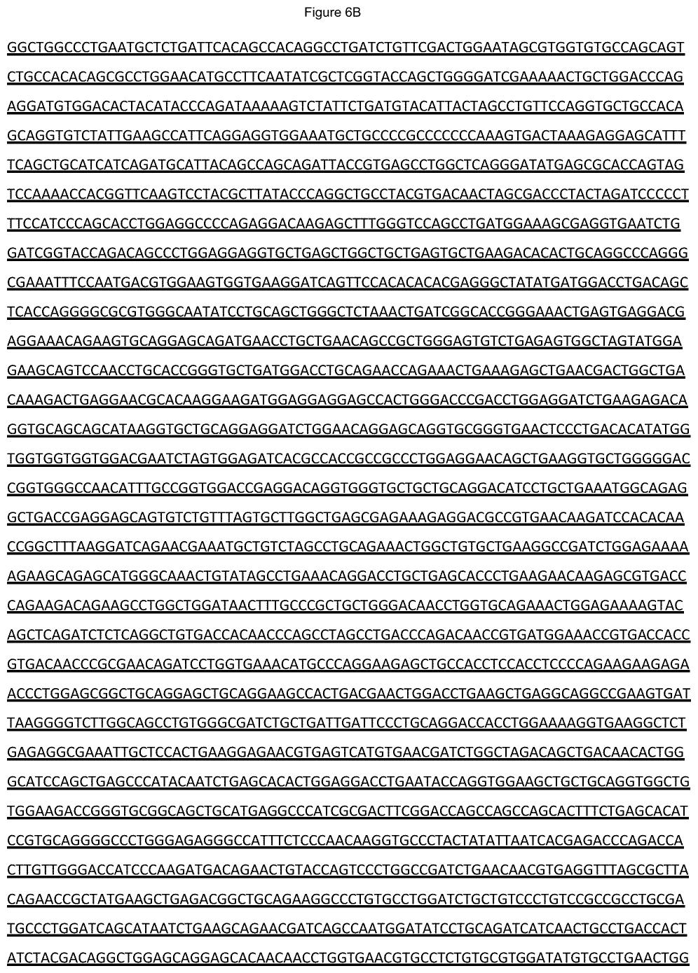

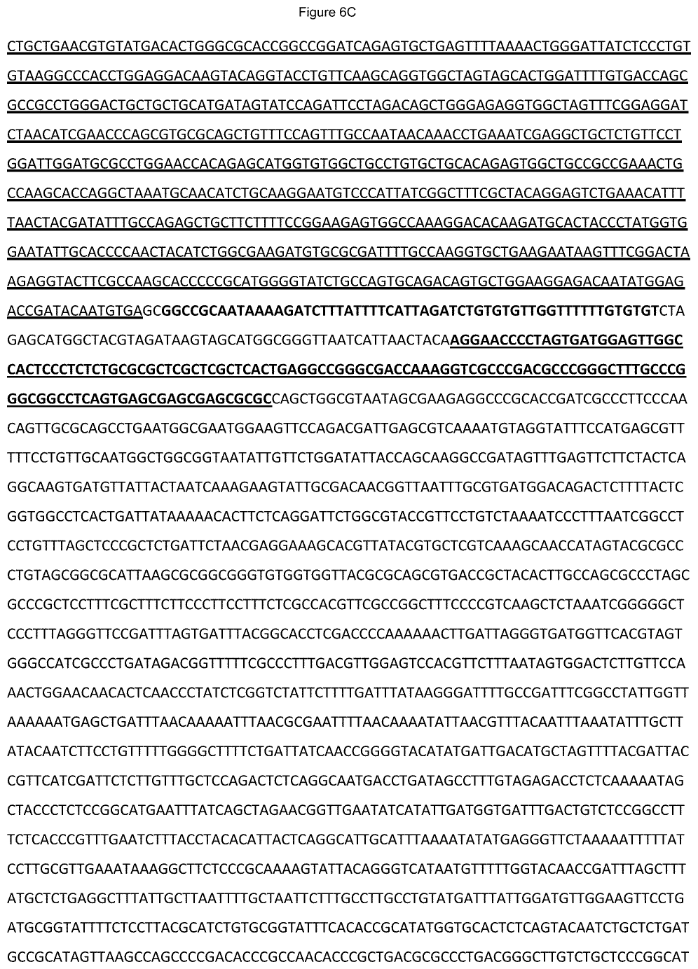

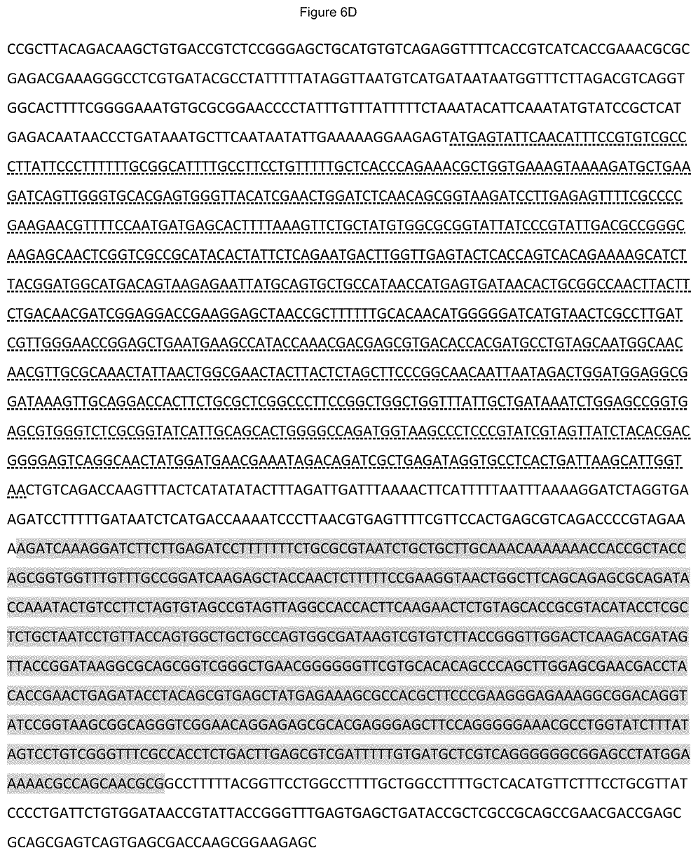

The pAAV.MCK.micro-dystrophin plasmid contained the human micro-dystrophin cDNA expression cassette flanked by AAV2 inverted terminal repeat sequences (ITR) (see FIG. 5). This sequence was encapsidated into AAVrh.74 virions. The molecular clone of the AAVrh.74 serotype was cloned from a rhesus macaque lymph node and is described in Rodino-Klapac et al. Journal of Tran. Med. 45 (2007).

D) Vector Production

The vector for the study described herein was produced utilizing a triple-transfection method of HEK293 cells, under research grade conditions. Characterization of the vector following production includes titer determination by qPCR with a supercoiled standard, endotoxin level determination (EU/mL) and a sterility assessment. The produced vector is analyzed by SDS-PAGE to verify banding pattern consistency with expected rAAV. The surrogate vector rAAVrh74.MCHK7.uDYS.FLAG used in these studies was constructed as described above with the addition of a C-terminus FLAG tag.

### Example 2

### Systemic Gene Delivery of rAAVrh74.MCHK7.Microdystrophin with Immunosuppression in Non-Human Primate Study

The principle goals for this study was to identify the optimal dose, duration, and immunosuppressing regimen, and to optimize gene expression after intravascular delivery of rAAV.rh74.MHCK7.micro-dystrophin. This study initiated using 5 cohorts of rhesus macaques (n=3 each cohort) with varying durations of immunosuppression prior to and post vector administration (Table 1). The rhesus macaques are referred to herein as “non-human primates” or NHPs.

Transgene Delivery Optimization Study in Nonhuman Primates

| | Immuno- | | | | | | |
|---|---|---|---|---|---|---|---|
| | suppression | | | | | | |
| Delivery | Treatment | | Animal | Dose | | Treatment | |
| Route | Plan | Duration | Strain | (vg/kg) | # | Endpoint | Analysis |
| IM | N/A | 30 days | mdx | 1 × 10¹¹ | 3 | 1 mo | H&E, Path. |
| (Potency) | | | | | | | Biodistribution, |
| | | | | | | | Western Blot, |
| | | | | | | | Chemistry, |
| | | | | | | | Immunology |
| IV | Prednisone | 1 day, pre- | Rhesus | — | 3 | 3 mo | H&E, Path. |
| (Efficacy) | | 30 days | Macaque | | | | Biodistribution, |
| | | post gene | | | | | Western Blot, |
| | | transfer | | | | | Chemistry, |
| | | | | | | | Immunology |
| IV | Prednisone | 1 day, pre- | Rhesus | 2 × 10¹⁴ | 3 | 3 mo | H&E, Path. |
| (Efficacy) | | 60 days | Macaque | | | | Biodistribution, |
| | | post gene | | | | | Western Blot, |
| | | transfer | | | | | Chemistry, |
| | | | | | | | Immunology |
| IV | Prednisone | 14 days, | Rhesus | 2 × 10¹⁴ | 3 | 3 mo | H&E, Path. |
| (Efficacy) | | pre-60 | Macaque | | | | Biodistribution, |
| | | days post | | | | | Western Blot, |
| | | gene | | | | | Chemistry, |
| | | transfer | | | | | Immunology |
| IV | Rituximab, | ++ | Rhesus | 2 × 10¹⁴ | 3 | 3 mo | H&E, Path. |
| (Efficacy) | Sirolimus, | | Macaque | | | | Biodistribution, |
| | Prednisone | | | | | | Western Blot, |
| | | | | | | | Chemistry, |
| | | | | | | | Immunology |
| IV | N/A | N/A | Rhesus | 2 ×10¹⁴ | 3 | 3 mo | H&E, Path. |
| (Control) | | | Macaque | | | | Biodistribution, |
| | | | | | | | Western Blot, |
| | | | | | | | Chemistry, |
| | | | | | | | Immunology |

++ Rituximab: Twice, 10-14 days pre-injection and once, day or injection.
Sirolimus: 3 days pre-injection through biopsy.
Prednisolone: 1 day pre-injection through 30 days post-injection.
IF: immunofluorescence;
H&E: hematoxylin and eosin staining;
Path: formal histopathology

In cohort 1, control macaques were dosed intravascularly with rAAVrh74.MHCK7.micro-dystrophin at 2×10¹⁴ vg/kg into cephalic or saphenous vein without the addition of immunosuppression (n=2). In all other cohorts, the macaques also received an intravascular injection of rAAVrh74.MHCK7.micro-dystrophin at 2×10¹⁴ vg/kg delivered into cephalic or saphenous vein, along with immunosuppression. In cohort 2, prednisone (2 mg/kg/day) was given orally 1 day prior to systemic gene transfer through 30 days post gene transfer (n=3). In Cohort 3, prednisone (2 mg/kg/day) was given orally 1 days prior to systemic gene transfer through 60 days post gene transfer (n=3). In Cohort 4, prednisone (2 mg/kg/day) was given orally 14 days prior to systemic gene transfer through 60 days post gene transfer (n=3).

Cohort 5 (n=3) investigated a triple immunosuppressing regimen. In this cohort, rituximab (750 mg/m2) was dosed via intravascular infusion for two dose sessions, 14 and 7 days before vector administration and a third dose on the day of vector administration prior to gene transfer. Rituximab may be administered a fourth time post infusion if antibodies are unresponsive to the first three doses. Sirolimus (4 mg/m²/day) was dosed orally 3 days before vector administration and continued until the reduction of antibodies. Prednisone was dosed orally (2 mg/kg/day) 1 day prior to vector administration through 30 days post vector administration.

After treatments, all cohorts underwent needle biopsies obtained from the Tibialis Anterior (TA) and/or gastrocnemius (gn). The biopsies were e collected prior to gene transfer and up to three tunes post gene transfer at 6, 8, and 12 weeks. Blood draws for immunology, CBC and chemistries were drawn at least biweekly.

The following hematology measurements were carried out on the blood samples: red blood cell (erythrocyte) count, hemoglobin, hematocrit, mean corpuscular volume, mean corpuscular hemoglobin, mean corpuscular hemoglobin, concentration, red cell distribution width, absolute reticulocyte count, platelet count white blood cell count, absolute basophil count, absolute large unstained cell count and blood smear.

The following clinical chemistry measurements were carried out on the blood samples: glucose, urea nitrogen, total protein, albumin, globulin, albumin:globulin ratio, total bilirubin, alanine aminotransferase, glutamate dehydrogenase, cholesterol, gamma glutamyltransferase, aspartate aminotransferase and alkaline phosphatase.

### Safety Profile and Transduction Efficiency

Total antibody response to AAVrh.74 (anti-AAVrh.74 antibodies) was similar across cohorts with no evidence of abnormal observations, except for one NHP from cohort 2 (NHP_03) that did not mount an antibody response to AAVrh.74. In addition, the NHPs from cohort 5, which despite being treated with a triple immunosuppressive regimen, demonstrated a similar antibody response to AAVrh.74 to that observed in the NHPs of cohorts 1-4. Adverse effects experienced by NHPs from cohorts 1-4 included transient elevated alanine transaminase (ALT) and aspartate transaminase (AST) liver enzymes. Two NHPs from cohort 1 (NHP_12, NHP_13), one NEW from cohort 3 (NHP_06) and one from cohort 4 (NHP_07) showed elevated ALT and AST liver enzymes at 12 weeks post-gene transfer. In regards to transduction efficiency, no statistically significant difference in vector genome copies (vg copies/μg DNA) was observed between NHP cohorts 1-5 at 12 weeks post-gene transfer (P>0.05).

### Example 3

### Administration with Therapeutic Plasma Exchange (TPE)

The principle goal for this study was to optimize both technique and gene expression after re-dosing with rAAVrh74.MHCK7.micro-dystrophin using therapeutic plasma exchanges (TPE) to remove pre-existing AAV antibodies and to evaluate redosing without the use of TPE.

Non-human primates previously injected with rAAVrh74.MHCK7.micro-dystrophin in the study described in Example 2 (from cohorts 2, 3 and 4) underwent 2-3 TPE during one apheresis procedure. Four and two weeks prior to TPE, primates underwent maximum blood draws (10% of primates body weight). Blood was preserved in ACDA solution for a maximum of 30 days and used on the day of TPE to prime the apheresis machine in order to prevent excessive blood loss during procedure. In addition, determination of AAVrh.74 binding antibody titers were measured to verify titers were greater than 1:400, the threshold of inclusion criteria in current clinical trials. Post TPE, 2×10¹⁴ vg/kg rAAVrh74.MHCK7.micro-dystrophin or rAAVrh74.MHCK7.micro-dystrophin.FLAG was delivered systemically via saphenous or cephalic vein.

These NHP also received prednisone (2 mg/kg) once per day 1 day prior to TPE and through 30 days post TPE and gene transfer. Blood draws for chemistries, CBC, ELISA and ELISpot assays were drawn pre-TPE, post TPE prior to re-dosing, and at least biweekly until endpoint. Endpoint was between 8 and 12 weeks post second gene transfer and included full necropsies. In order to evaluate efficacy, western blot and qPCR for vector genomes and FLAG immunofluorescence were carried out.

Additionally, non-human primates previously injected while on the immunosuppressing regimen described in Example 2 (cohort 5) were redosed with rAAVrh74.MHCK7.micro-dystrophin without the use of TPE to lower pre-existing antibodies towards AAVrh.74. Specifically, Rituximab (750 mg/m²/day) was delivered intravascularly (IV) 7 and 14 days pre gene transfer, once the day of injection, and once after gene transfer, sirolimus (4 mg/m²/day) was delivered 3 days pre gene transfer and continued until the conclusion of the study. Sirolimus levels were monitored via blood collection varying between 3 to 14 ng/mL. Blood draws for chemistries, CBC, Sirolimus blood levels, ELISA and ELISpot assays were drawn at least biweekly until endpoint. Endpoint was between 8 and 12 weeks post second gene transfer and included full necropsies. Observations of each animal were performed daily. NHP body weight was monitored bi-weekly and dosing of immunosuppression drugs adjusted accordingly.

### Therapeutic Plasma Exchange Procedure:

In the TPE process, whole blood was removed via vascular access and subsequently spun through a centrifuge within the apheresis machine, where the plasma (antibodies) was removed. Red blood cells were delivered back along with a replacement fluid (Human Albumin) for maintenance fluid for the primate. Due to non-human primates' small size (less than 10 kg), priming the apheresis machine prior to plasma exchanges with preserved blood was performed to ensure safety and reduce the amount of blood withdrawn from primates. 28 and 14 days prior to apheresis, a maximum blood collection (10% of non-human primates circulating blood) was performed. Collected whole blood was preserved and stored for no more than 30 days in anticoagulant acid-citrate-dextrose (ACDA) solution at 4° C. Additionally, NHP were provided extra iron-rich supplements and enrichment. On the day of apheresis, non-human primates were sedated intramuscularly with telazol (3-6 mg/kg), intubated, and secured to a heated procedure table. Anesthetic maintenance was achieved with isoflurane in oxygen 1-4%. Angiocatheters were placed in both legs (saphenous vein), with one access port to withdraw whole blood and second in the opposite leg to re-deliver red blood cells and replacement fluid. Additional catheters were placed in arms (cephalic vein) for support fluid and blood draws throughout the procedure. After vascular access was obtained, the animals were dosed with heparin (50-100 U/kg) to maintain adequate blood flow and prevent clotting during apheresis. NHP were monitored using temperature, ECG, and respirations to determine proper anesthetic plane.

NHP were connected to COBE Spectra apheresis machine through catheters and machine was primed with pre-collected blood, as mentioned above. One total plasma exchange equates to the entire amount of circulating blood being removed and replaced one time. Two to three plasma exchanges were performed to achieve an estimated antibody removal of 98%. Blood was collected after each completed exchange for blood chemistry analysis and serum antibody testing. Immediately post plasma exchanges, the NHP was disconnected from apheresis unit and systemically re-dosed with rAAVrh74.MHCK7.micro-dystrophin. Post vector delivery, all catheters were removed, pressure was provided to control bleeding. Animals were monitored until fully ambulatory.

### Necropsy Analysis

For necropsies, the NHP were dosed with Euthasol (1 mL/101 b) at the endpoint (which may occur between 8-12 weeks post redosing). Blood was collected and whole blood was sent for complete blood count (CBC) analysis, sirolimus testing levels, and serum chemistries. Tissues were then collected and sent for analysis by an independent veterinary histopathologist and gene and protein expression are analyzed to evaluate efficacy and toxicity.

In order to evaluate gene expression optimization, the pre and post muscle biopsies from NHP dosed and re-dosed intravenously with 2×10¹⁴ vg/kg rAAVrh74.MHCK7.micro-dystrophin or rAAVrh74.MHCK7.micro-dystrophin.FLAG were collected. DNA from the pre and all post muscle tissues were extracted for real-time quantitative qPCR to detect specific sequences of vector DNA. Protein was extracted from all muscles collected and western blots were performed to detect micro-dystrophin protein (138 kD) compared to the pre-biopsy tissues. Additionally, naive full-length dystrophin (427 kD) was used as a normal control and quantitatively compared to micro-dystrophin protein as an outcome measure as gene expression. Immunofluorescence staining to observe the presence of FLAG expression were performed on rAAVrh74.MHCK7.micro-dystrophin.FLAG infused primates.

In order to evaluate safety of the immunosuppression regimen, vector administration, and redosing, blood draws took place at baseline and bi-weekly until endpoint. Serum chemistries, CBC, and sirolimus levels were monitored bi-weekly throughout both aims of the study. ELISpot analysis was utilized to evaluate T cell response to both AAVrh.74 peptides and microdystrophin peptides. Finally, anti-AAVrh.74 antibody response was monitored bi-weekly throughout both aims of the study.

The following hematology measurements were carried out on the blood samples: red blood cell (erythrocyte) count, hemoglobin, hematocrit, mean corpuscular volume, mean corpuscular hemoglobin, mean corpuscular hemoglobin, concentration, red cell distribution width, absolute reticulocyte count, platelet count white blood cell count, absolute basophil count, absolute large unstained cell count and blood smear.

The following clinical chemistry measurements were carried out on the blood samples: glucose, urea nitrogen, total protein, albumin, globulin, albumin:globulin ratio, total bilirubin, alanine aminotransferase, glutamate dehydrogenase, cholesterol, gamma glutamyltransferase, aspartate aminotransferase and alkaline phosphatase.

### Results

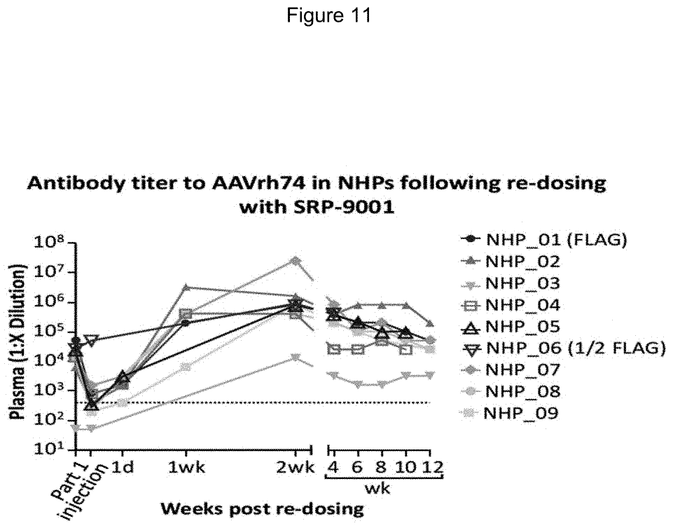

Total antibody titer against AAVrh.74 in NHPs prior to TPE and following TPE (before redosing with rAAVrh74.MHCK7.micro-dystrophin) are provided in the table below. FIG. 11 provides the antibody titer to AAV74 in NHPs following re-dosing with rAAVrh74.MHCK7.micro-dystrophin. The number of TPC cycles that can be performed in NHPs is limited due to the lack of donor blood available. In humans, multiple rounds of TPE can be administered. The titers detected in Example 2 were obtained (*) 12 weeks post initial gene transfer. The titer detected in Example 3 were obtained (+) prior to re-dose injection of rAAVrh74.MHCK7.micro-dystrophin. NHP_03 was re-dosed without prior TPE due to lack of antibody response to AAVrh.74. NHP_06 only underwent 0.5 cycles of TPE due to small size and poor vascular access.

| | Titer after Gene | | |
|---|---|---|---|
| | Therapy | Titer after TPE | Number of TPE |
| NHP (cohort) | (Example 2)* | (Example 3) + | Cycles |
| NHP_01(2) | 1:51200 | 1:800 | 2.5 |
| NHP_02(2) | 1:6400 | 1:400 | 3 |
| NHP_03(2) | 1:50 | NA | NA |
| NHP_04(3) | 1:12800 | 1:800 | 3 |
| NHP_05(3) | 1:25600 | 1:400 | 3 |
| NHP_06(3) | 1:25600 | NA | 0.5 |
| NHP_07(4) | 1:12800 | 1:1600 | 3 |
| NHP_08(4) | 1:12800 | 1:200 | 3 |
| NHP_09(4) | 1:12800 | 1:200 | 3 |

The TPE procedure was generally well tolerated. There were no abnormal immunological observations as assessed by IFN-□ spot forming cell (SCF) levels against AAVrh.74 and micro-dystrophin peptides from peripheral blood mononuclear cells. Re-dosing following TPE resulted in increased liver enzyme levels (ALT/AST) in the following NHPs: NHP_01 and NHP_02, cohort 2; NHP_04, cohort 3; NHP_08 and NHP_09, cohort 4). This was resolved with continued prednisone daily administration.

NHPs from cohort 5 did not receive TPE due to incompatibility with previous treatment with rituximab and two NHPs (NHP_10, NHP_11) were re-dosed. Cohort 5 had the total antibody titer to AAVrh.74 higher than 1:51,200 before re-dosing. NHPs re-dosed at high antibody titer (cohort 5) experienced the following adverse events: increased heart rate and ventilation rate, vomiting, rash near delivery site, pale, and shallow breathing; resolved after administration of diphenhydramine and dexamethasone.

Seven NHPs underwent 2-3 consecutive cycles of TPE, resulting in reduced levels of circulating antibodies against AAVrh.74. Immediately following TPE, NHPs were successfully re-dosed with rAAVrh74.MHCK7.micro-dystrophin. In two NHPs from cohort 4 (NHP_08 and NHP_09) antibody titers of 1:200 were achieved.

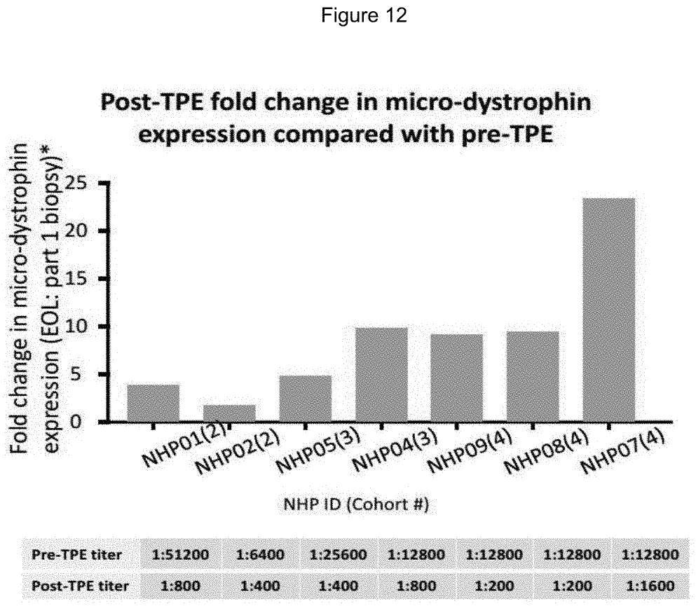

As shown in FIG. 12, increased expression of micro-dystrophin protein was observed in tissue samples from all NHPs re-dosed with rAAVrh74.MHCK7.micro-dystrophin after TPE when compared with expression pre-TPE from biopsy at week 12 (as described in Example 2). Increased micro-dystrophin protein expression was observed in skeletal muscle (e.g. gastrocnemius), heart and diaphragm.

### Example 4

The trials and studies described in Examples 2 and 3 above are alternatively carried out utilizing the rAAVrh74.MHCK7.micro-dystrophin construct set forth in SEQ ID NO: 9; as set forth in SEQ ID NO: 8, nucleotides 1-4977; or as set forth in SEQ ID NO: 6; nucleotides 56-5022.

### Example 5

### Gene Therapy for DMD with Pre-Existing AAVrh.74 Antibodies Following Therapeutic Plasma Exchange (TPE

A phase 1 clinical trial is carried out in humans to investigate gene therapy of DMD in patients with pre-existing AAVrh.74 antibodies following Therapeutic Plasma Exchange (TPE). It is hypothesized that five cycles of TPE will lower binding antibodies to AAVrh.74 (also referred to herein as “anti-AAVrh.74 antibodies”), allowing safe and efficient transduction of muscle using AAVrh74.MHCK7.micro-dys to achieve mean expression levels >50% compared to baseline.

The study objective and primary outcome is the safe delivery of rAAV carrying the micro-dystrophin gene (AAVrh74.MHCK7.micro-dystrophin). The secondary objective is micro-dys gene expression in the muscle of the subject suffering from DMD and clinical improvement using the North Star Ambulatory Assessment for muscular dystrophy (NSAA) as a functional outcome measure.

The treatment plan is a two-step (week) protocol that merges safety and efficacy (Table 1) to first reduce AAVrh.74 antibodies by apheresis over a 10-day schedule followed by intravenous delivery of AAVrh74.MHCK7.micro-dys. In week 1, TPE is administered other day for 3 day, such as Monday (day −9 relative to the infusion day of the gene therapy), Wednesday (day −7), and Friday (day−5). In week 2, TPE is administered every other day for two days followed by intravenous infusion of AAVrh74.MHCK7.micro-dystrophin such as Monday (day−2), Wednesday (day 1) with delivery of AAVrh74.MHCK7.micro-dystrophin (Day 1) that same day following TPE. The patient is brought to the outpatient apheresis unit according to the schedule in Table 1. The patient is admitted to the pediatric intensive care unit (PICU) for in-patient gene therapy infusion on day 1 with planned discharge on day 2.

TABLE 1
APHERESIS SCHEDULE

| | Monday | Tuesday | Wed | Thurs | Fri | Sat | Sun |
|---|---|---|---|---|---|---|---|
| Week | Apheresis | Binding | Apheresis | | Apheresis | | |
| 1 | (#1) | Abs | (#2) | | (#3) | | |
| | *Labs | AAV** | *Labs | | *Labs | | |
| Week | Apheresis | Binding | Apheresis | | **** Stand In | | |
| 2 | (#4) | Abs | (#5) | | Schedule | | |
| | *Labs | AAV** | *Labs | | | | |
| | | | ***Gene | | | | |
| | | | Therapy | | | | |

*Labs prior to procedure include fibrinogen and CBC with differential and platelets; ionizied calcium periodically measured during procedure
**AAVrh.74 Antibody Level <1:100 to permit gene delivery
*** Gene Delivery
****Stand-In Schedule

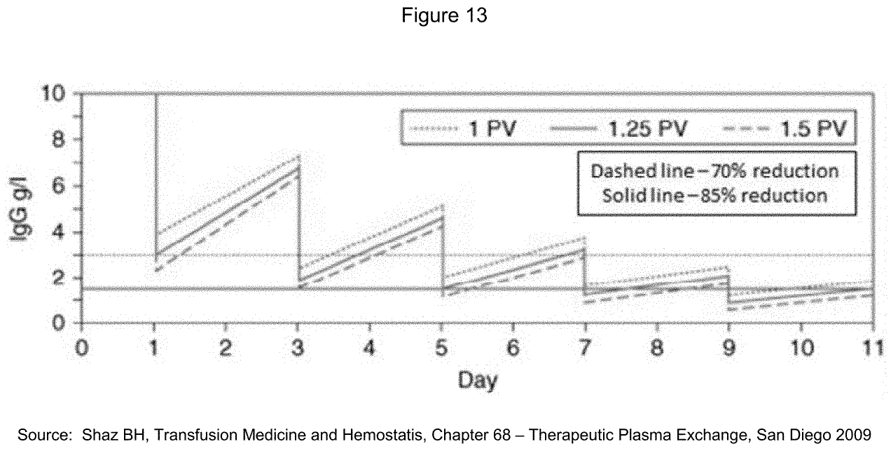

This protocol has been developed based on the kinetics of IgG antibodies, showing that after 48 hours there is rebound, gaining back roughly 50-60% of antibodies removed from the previous procedure; thus supportive of TPE over a 10-day course every other day followed by gene delivery of the final day of TPE (designated Day 1 in relation to gene therapy) (FIG. 13 and see Exchange Volumes). The timing of these procedures follows a similar course for other known antibody-mediated medical conditions (Padmanabhan et al., J Clin Apheresis 34:171-354, 2019).

Since this protocol is developed for a new indication, it is appropriate to follow previously established guidelines that repeatedly recommend 5-6 (Padmanabhan et al., J Clin Apheresis 34:171-354, 2019) to 5-7 treatments (Pham et al., Transfusion and Apheresis Science 58:237-246, 2019). Additional support that applies to the subjects in this protocol taking prednisone for immune suppression states that “If, as a result of concurrent immunosuppressive therapy, one assumes a negligible production rate of immunoglobulin, and the rate of extravascular to intravascular equilibration to be approximately 1 to 2 percent per hour, then five separate procedures over 7 to 10 days are required to remove 90 percent of the total initial body immunoglobulin burden. Additional treatments may be required if new antibody production occurs. (Fridey & Kaplan Therapeutic apheresis (plasma exchange or cytapheresis): Indications and technology. American Society for Apheresis Guidelines UPTODATE May 2020 (available on-line) (Kaplan et al. J Clin Apheresis 28:3-10, 2013)

### Protocol

Patient Characteristics for this Protocol:

Inclusion Criteria
Ambulatory Male subjects, any ethnic group, ages 4-10 inclusive at time of screening
Confirmed DMD frameshift or premature stop codons gene mutations
CK >1000 U/L
Below 95^{th} percentile predicted for age on 100 m walk test indicative of symptomatic disease
Ability to cooperate with motor assessment testing
Weakness demonstrated based on history of difficulty running, jumping and climbing stairs
Stable dose equivalent of oral corticosteroids for at least 12 weeks before screening and the dose is expected to remain constant (except for potential modifications to accommodate changes in weight) throughout the study.
Patients with AAVrh.74 antibody titers >1:400 as determined by ELISA immunoassay at baseline screening (day −40 to −10)

Exclusion Criteria
Signs of Cardiomyopathy, including ECHO with LVEF <40%
Serological evidence of human immunodeficiency virus (HIV) infection, or hepatitis B or C infection
Diagnosis of (or ongoing treatment for) an autoimmune disease
Abnormal laboratory values considered clinically significant (gamma-glutamyl transferase, >3×upper limit of normal, bilirubin ≥3 mg/dL, creatinine ≥1.8 mg/dL, hemoglobin <8 or >18 g/dL, white blood cell count >18,500 per cmm, platelets ≤50,000 per microliter.
Concomitant illness or requirement for chronic drug treatment that, in the opinion of the PI, creates unnecessary risks for gene transfer.
Severe infection (e.g., pneumonia, pyelonephritis, or meningitis) within 4 weeks before.
Has received any investigational medication (other than corticosteroids) or exon skipping medications (including EXONDYS 51®), experimental or otherwise, within 6 months of screening.
Has received any type of gene therapy, cell-based therapy (e.g., stem cell transplantation), or CRISPR/Cas9 therapy.
Family does not want to disclose patient's study participation with primary care physician and other medical providers.

### Screening/Baseline Period

The Screening/Baseline period is up to 4 weeks before Day−9. After obtaining informed consent, patients are evaluated for eligibility. Screening includes collection of demographics and medical history, vital sign measurements, a physical examination, electrocardiogram (ECG), and an echocardiogram (ECHO) and cardiac MRI. Blood and urine samples are collected for clinical and safety laboratory assessments. Blood samples are also be collected for hepatitis B and C, human immunodeficiency virus (HIV), and antibodies to AAVrh.74 and antigen-specific T-cells to AAVrh.74 capsid and micro-dystrophin. A pretreatment muscle biopsy involves the gastrocnemius muscle, or a muscle selected by the Principal Investigator (PI) and is performed after eligibility but before Day −9. The parent/caregiver is asked to complete PROMIS questionnaires. Physical functional assessments include North Star Ambulatory Assessment for Muscular Dystrophy (NSAD), and timed-functional tests including rising from the floor, ascending 4 standard steps, the 10-meter and 100-meter walk/run tests.

### Therapeutic Plasma Exchange

TPE involves removal of patient plasma and replacement with 5% albumin. Fresh frozen plasma (FFP) can be used during any of the TPE procedures if needed for patient safety. Plasma removed during plasma exchange is not be used for transfusion to another individual, according to regulations from the US Food and Drug Administration (FDA).

### Protocol

TPE is carried out through peripheral veins when possible. More likely patients will have a tunneled central line put in by the interventional radiologist. Parents are instructed on how to care for the catheter in between appointments.

### Exchange Volumes—

Plasma exchange of 1.0 to 1.5 plasma volume exchanges is performed per procedure. A single plasma exchange lowers plasma macromolecule levels by 63% (J Clin Apheresis 2019; 34:171-354). IgG antibodies that are distributed in both the intravascular and extravascular compartments require multiple exchanges to decrease total body stores and are usually performed every other day to allow redistribution between both compartments. Exchange of the first 1.0 to 1.5 plasma volumes removes the highest volume of the target substance, with diminishing amounts removed with each subsequent exchange. For each single plasma volume exchange the same volume of replacement fluid is used. For this protocol 5% albumin is used as replacement fluid.

Immunosuppressive regimens are advised to obtain sustained response. Without immune suppression there may be rebound following exchange but all the DMD boys participating in this program will be on glucocorticoids as standard treatment for disease. During the experimental protocol for plasma exchange, patients are put on glucocorticoid (prednisone 1 mg/kg, or equivalent corticosteroid), one day prior to TPE. Patients will maintain this dose for at least 60 days post gene delivery after the gene transfer unless earlier tapering is judged by the PI to be in the best interest of the patient. (Table 1).

In further interest of the patient, an alternate plan (referred to as the “Stand-In Schedule”) is provided that might delay gene delivery and provide for #7 TPE to allow gene therapy to proceed. If the target AAVrh.74 titer is not reached on day −1 following apheresis #4, TPE #5 will be carried out but not proceed with gene delivery. AAV antibody levels are obtained on day 2 (Thurs) followed by apheresis, TPE #6 and gene delivery on Fri. If, however, titers have not reached target of 1:100 on Thurs (day 2) we will not proceed to TPE or gene delivery on Fri.

### Gene Delivery

This is a 52 week open-label clinical trial. Six to twelve patients meeting eligibility requirements are enrolled and receive IV AAVrh74.MHCK7.micro-dystrophin. On Day −1, as an outpatient, physical examination is carried out and vital signs, blood samples and urine samples are collected. On Day 1, the patient is admitted to the hospital. The fifth and final TPE is carried out in the morning and later the same day receive AAVrh74.MHCK7.micro-dystrophin administered IV over 1 to 2 hours according to the Study Operations Manual used for prior protocols approved by IRB and FDA. On the day after the infusion (Day 2), patients receive a physical examination, have vital signs collected, and provide blood and urine samples before being discharged.

Patients are followed for 452 weeks and then proceed to a long term follow up study for 5 years. Patients complete clinic follow-up visits post gene delivery: Weeks 1, 2, 4, 6, 8, 10, 12, 24, 36, and 48 (relative to the infusion on Day 1). In addition, at Weeks 3 and 5, patients complete follow-up visits for assessment of liver function tests. All patients have a muscle biopsy performed at Week 12. The biopsy involves the gastrocnemius muscle, or a muscle selected by the PI.

Safety is assessed by monitoring of treatment-emergent adverse events (TEAEs), SAEs, and select laboratory assessments.

### Example 6

### Sandwich ELISA Determination of Antibodies in Sera

Materials for the sandwich ELISA assay are as follow:
Capture Antibody=anti-AAVrh.74 mAb
Test sample=serum or plasma, will serve in detection
Antigen=AAVrh.74 capsid
Blocking Solution=5% dry milk, 1% goat serum, 100 mL PBS
Wash Buffer=0.05% PBS-Tween
Positive Control=serum known to have anti-AAVrh.74 antibodies
Secondary Antibody=anti-human-HRP conjugated antibody
Substrate=TMB
Stop Solution=Sulfuric Acid

Method

All wells of a 96-well plate are coated overnight with the capture antibody diluted in carbonate buffer at 4 C. The content is discarded, and the plate is blocked with the blocking solution for 1 h at 37 C. Blocking solution is discarded to add AAVrh.74 capsid in duplicate on to the capture antibody coated wells. Additionally, carbonate buffer is added to duplicate wells to determine background value. Unbound capsid is discarded and the test serum is added at a starting dilution of 1:25 in blocking solution and serially diluted. Positive control is diluted in blocking solution at a 1:400 dilution. Plate is washed with wash buffer, followed by secondary incubation at a dilution of 1:10,000 in blocking solution. Plate is washed and buffer is discarded, and substrate is added followed by concluding the assay with sulfuric acid. The plate absorbance is read at 450 nm.

### Analysis and Results

Absorbance ratio is determined by subtracting the average optical density (OD) of the non-antigen coated wells from the average OD of the antigen coated wells and dividing by the average (OD) of the non-antigen coated wells. A ratio of ≥2.00 is considered a positive antibody response. The endpoint titer is determined by identifying the last serum dilution yielding a ratio of ≥2.00. The antibody cutoff is defined at a serum dilution of >1:400.

### Example 7

### Indirect ELISA: Determination of Anti-AAVrh.74 Antibodies in Sera

Materials
Antigen=AAVrh.74 capsid
Blocking Solution=5% dry milk, 1% goat serum, 100 mL PBS without calcium and magnesium
Wash Buffer=0.05% PBS-Tween
Primary Antibody=human test serum or plasma
Positive Control=anti AAVrh74 mAb or optionally sera (or plasma) containing anti-rh74 antibodies
Negative Control: Serum from subjects without anti-AAVrh74 antibodies can be included as a negative control
Secondary Antibody=anti-human antibody
Substrate=3, 3′, 5,5′-tetramethylbenzidine (TMB)
Stop Solution=Sulfuric Acid

Method

Duplicates wells within a 96 well plate coated overnight with antigen at a concentration of 2×10⁹ vg/well diluted in carbonate buffer. Additionally, carbonate buffer is added to duplicate wells to determine background. Antigen is discarded and wells are blocked with blocking solution. Blocking solution is discarded and primary antibody (test serum) is added at a starting dilution of 1:25 in blocking solution and serially diluted. Positive control is diluted in blocking solution at a 1:400 dilution if using serum or for example, at 1:1,000-1:10,000 dilution if using AAVrh.74 mAb. It is recognized that the particular dilution of the anti-AAVrh.74 mAb can be optionally determined utilizing the serum or plasma containing anti-AAVrh.74 antibodies as a reference. The plate is washed with wash buffer, followed by secondary incubation at a dilution of 1:10,000 in blocking solution. The plate is washed and buffer is discarded, and substrate is added, followed by concluding the assay with sulfuric acid. The plate absorbance is read at 450 nm.

Analysis and Results

Absorbance ratio is determined by subtracting the average optical density (OD) of the non-antigen coated wells from the average OD of the antigen coated wells and dividing by the average (OD) of the non-antigen coated wells. A ratio of ≥2.00 is considered a positive antibody response, and a plasma or serum sample with such positive response is considered to be seropositive. The endpoint titer is determined by identifying the last serum dilution yielding a ratio of ≥2.00. The antibody titer cutoff is defined at a serum dilution of >1:400. In other words, a subject with a ratio of ≥2.00 at a serum dilution of 1:400 would be considered seropositive and excluded from receiving rAAVrh.74-based gene therapy.

### Example 8

### scAAVrh.74.MHCK7.hSGCB Construction

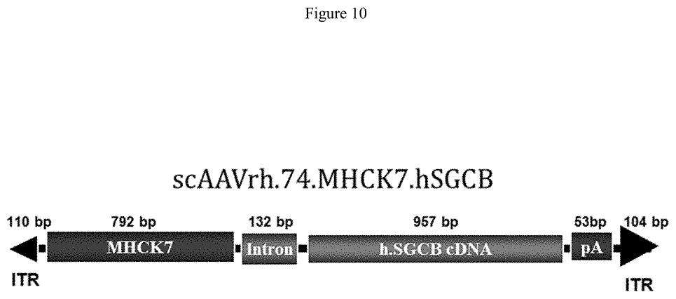

The transgene cassette containing a codon-optimized full-length human SCGB cDNA as shown in FIG. 10 was constructed. The cassette includes a consensus Kozak sequence (CCACC), an SV40 chimeric intron, a synthetic polyadenylation site, and the muscle-specific MHCK7 used to drive expression of the cassette. This is an MCK based promoter which utilizes a 206-bp enhancer taken from ˜1.2 kb 5′ of the transcription start site within the endogenous muscle creatine kinase gene with a proximal promoter (enh358MCK, 584-bp). The cassette was packaged into a self-complementary (sc) AAVrh.74 vector that is 93% homologous to AAV8. AAVrh.74 has been shown in mice and non-human primates to be safe and effective, particularly in crossing the vascular barrier when delivered to muscle through the circulation.

### REFERENCES

1. Hoffman, E. P., Brown, R. H., Jr. & Kunkel, L. M. Dystrophin: the protein product of the Duchenne muscular dystrophy locus. Cell 51, 919-928 (1987).
2. Straub, V. & Campbell, K. P. Muscular dystrophies and the dystrophin-glycoprotein complex. Curr Opin Neurol 10, 168-175 (1997).
3. Sacco, A., et al. Short telomeres and stem cell exhaustion model Duchenne muscular dystrophy in mdx/mTR mice. Cell 143, 1059-1071 (2010).
4. Wallace, G. Q. & McNally, E. M. Mechanisms of muscle degeneration, regeneration, and repair in the muscular dystrophies. Annu Rev Physiol 71, 37-57 (2009).
5. Zhou, L. & Lu, H. Targeting fibrosis in Duchenne muscular dystrophy. J Neuropathol Exp Neurol 69, 771-776 (2010).
6. Desguerre, I., et al. Endomysial fibrosis in Duchenne muscular dystrophy: a marker of poor outcome associated with macrophage alternative activation. J Neuropathol Exp Neurol 68, 762-773 (2009).
7. DiPrimio, N., McPhee, S. W. & Samulski, R. J. Adeno-associated virus for the treatment of muscle diseases: toward clinical trials. Curr Opin Mol Ther 12, 553-560 (2010).
8. Mendell, J. R., et al. Sustained alpha-sarcoglycan gene expression after gene transfer in limb-girdle muscular dystrophy, type 2D. Ann Neurol 68, 629-638 (2010).
9. Mendell, J. R., et al. Limb-girdle muscular dystrophy type 2D gene therapy restores alpha-sarcoglycan and associated proteins. Ann Neurol 66, 290-297 (2009).
10. Mendell, J. R., et al. A phase 1/2a follistatin gene therapy trial for becker muscular dystrophy. Molecular therapy: the journal of the American Society of Gene Therapy 23, 192-201 (2015).
11. Carnwath, J. W. & Shotton, D. M. Muscular dystrophy in the mdx mouse: histopathology of the soleus and extensor digitorum longus muscles. J Neurol Sci 80, 39-54 (1987).
12. Coulton, G. R., Morgan, J. E., Partridge, T. A. & Sloper, J. C. The mdx mouse skeletal muscle myopathy: I. A histological, morphometric and biochemical investigation. Neuropathol Appl Neurobiol 14, 53-70 (1988).
13. Cullen, M. J. & Jaros, E. Ultrastructure of the skeletal muscle in the X chromosome-linked dystrophic (mdx) mouse. Comparison with Duchenne muscular dystrophy. Acta Neuropathol 77, 69-81 (1988).
14. Dupont-Versteegden, E. E. & McCarter, R. J. Differential expression of muscular dystrophy in diaphragm versus hindlimb muscles of mdx mice. Muscle Nerve 15, 1105-1110 (1992).
15. Stedman, H. H., et al. The mdx mouse diaphragm reproduces the degenerative changes of Duchenne muscular dystrophy. Nature 352, 536-539 (1991).
16. Deconinck, A. E., et al. Utrophin-dystrophin-deficient mice as a model for Duchenne muscular dystrophy. Cell 90, 717-727 (1997).
17. Grady, R. M., et al. Skeletal and cardiac myopathies in mice lacking utrophin and dystrophin: a model for Duchenne muscular dystrophy. Cell 90, 729-738 (1997).
18. Love, D. R., et al. An autosomal transcript in skeletal muscle with homology to dystrophin. Nature 339, 55-58 (1989).
19. Tinsley, J. M., et al. Primary structure of dystrophin-related protein. Nature 360, 591-593 (1992).
20. Tinsley, J., et al. Expression of full-length utrophin prevents muscular dystrophy in mdx mice. Nat Med 4, 1441-1444 (1998).
21. Squire, S., et al. Prevention of pathology in mdx mice by expression of utrophin: analysis using an inducible transgenic expression system. Hum Mol Genet 11, 3333-3344 (2002).
22. Rafael, J. A., Tinsley, J. M., Potter, A. C., Deconinck, A. E. & Davies, K. E. Skeletal muscle-specific expression of a utrophin transgene rescues utrophin-dystrophin deficient mice. Nat Genet 19, 79-82 (1998).
23. Zhou, L., et al. Haploinsufficiency of utrophin gene worsens skeletal muscle inflammation and fibrosis in mdx mice. J Neurol Sci 264, 106-111 (2008).
24. Gutpell, K. M., Hrinivich, W. T. & Hoffman, L. M. Skeletal Muscle Fibrosis in the mdx/utrn+/− Mouse Validates Its Suitability as a Murine Model of Duchenne Muscular Dystrophy. PloS one 10, e0117306 (2015).
25. Rodino-Klapac, L. R., et al. Micro-dystrophin and follistatin co-delivery restores muscle function in aged DMD model. Human molecular genetics 22, 4929-4937 (2013).
26. Nevo, Y., et al. The Ras antagonist, farnesylthiosalicylic acid (FTS), decreases fibrosis and improves muscle strength in dy/dy mouse model of muscular dystrophy. PloS one 6, e18049 (2011).
27. Rodino-Klapac, L. R., et al. A translational approach for limb vascular delivery of the micro-dystrophin gene without high volume or high pressure for treatment of Duchenne muscular dystrophy. J Transl Med 5, 45 (2007).
28. Mulieri, L. A., Hasenfuss, G., Ittleman, F., Blanchard, E. M. & Alpert, N. R. Protection of human left ventricular myocardium from cutting injury with 2,3-butanedione monoxime. Circ Res 65, 1441-1449 (1989).
29. Rodino-Klapac, L. R., et al. Persistent expression of FLAG-tagged micro dystrophin in nonhuman primates following intramuscular and vascular delivery. Molecular therapy: the journal of the American Society of Gene Therapy 18, 109-117 (2010).
30. Grose, W. E., et al. Homologous recombination mediates functional recovery of dysferlin deficiency following AAV5 gene transfer. PloS one 7, e39233 (2012).
31. Liu, M., et al. Adeno-associated virus-mediated microdystrophin expression protects young mdx muscle from contraction-induced injury. Mol Ther 11, 245-256 (2005).
32. Harper, S. Q., et al. Modular flexibility of dystrophin: implications for gene therapy of Duchenne muscular dystrophy. Nature medicine 8, 253-261 (2002).
33. Rodino-Klapac, L. R., et al. Persistent expression of FLAG-tagged micro dystrophin in nonhuman primates following intramuscular and vascular delivery. Mol Ther 18, 109-117 (2010).
34. Salva, M. Z., et al. Design of tissue-specific regulatory cassettes for high-level rAAV-mediated expression in skeletal and cardiac muscle. Mol Ther 15, 320-329 (2007).
35. Sondergaard, P. C., et al. AAV.Dysferlin Overlap Vectors Restore Function in Dysferlinopathy Animal Models. Annals of clinical and translational neurology 2, 256-270 (2015).
36. De, B. P., et al. High levels of persistent expression of alpha1-antitrypsin mediated by the nonhuman primate serotype rh.10 adeno-associated virus despite preexisting immunity to common human adeno-associated viruses. Mol Ther 13, 67-76 (2006).
37. Rodino-Klapac, L. R., et al. A translational approach for limb vascular delivery of the micro-dystrophin gene without high volume or high pressure for treatment of Duchenne muscular dystrophy. Journal of translational medicine 5, 45 (2007).
38. Bulfield et al., X chromosome-linked muscular dystrophy (mdx) in the mouse. Proc Natl Acad Sci USA. 1984; 81(4): 1189-1192.
39. Sicinski et al., The molecular basis of muscular dystrophy in the mdx mouse: a point mutation. Science. 1989 30; 244(4912):1578-80

| SEQUENCE LISTING | |
|---|---|
| SEQ ID NO: 1 | |
| <211> 3579 | |
| <212> DNA | |
| <213> Homo sapiens | |
| <400> 1 | |
| atgctgtggt gggaggaggt ggaggattgt tatgaaaggg aggacgtgca gaagaagact | 60 |
| tttaccaagt gggtgaacgc tcagttcagc aaatttggga agcagcacat cgagaatctg | 120 |
| ttttccgacc tgcaggatgg gagacggctg ctggatctgc tggaaggact gactggccag | 180 |
| aagctgccca aagagaaggg gagcactagg gtgcacgccc tgaacaacgt gaacaaagct | 240 |
| ctgagagtgc tgcagaacaa caacgtggat ctggtgaata ttggcagtac tgatatcgtg | 300 |
| gacgggaacc acaaactgac actgggcctg atctggaaca ttattctgca ctggcaggtg | 360 |
| aaaaatgtga tgaagaacat catggccggg ctgcagcaga ccaattccga gaagatcctg | 420 |
| ctgtcttggg tgcggcagag cacccgcaac tatccccagg tgaacgtgat taacttcact | 480 |
| acatcctgga gcgacgggct ggccctgaat gctctgattc acagccacag gcctgatctg | 540 |
| ttcgactgga atagcgtggt gtgccagcag tctgccacac agcgcctgga acatgccttc | 600 |
| aatatcgctc ggtaccagct ggggatcgaa aaactgctgg acccagagga tgtggacact | 660 |
| acatacccag ataaaaagtc tattctgatg tacattacta gcctgttcca ggtgctgcca | 720 |
| cagcaggtgt ctattgaagc cattcaggag gtggaaatgc tgccccgccc ccccaaagtg | 780 |
| actaaagagg agcattttca gctgcatcat cagatgcatt acagccagca gattaccgtg | 840 |
| agcctggctc agggatatga gcgcaccagt agtccaaaac cacggttcaa gtcctacgct | 900 |
| tatacccagg ctgcctacgt gacaactagc gaccctacta gatccccctt tccatcccag | 960 |
| cacctggagg ccccagagga caagagcttt gggtccagcc tgatggaaag cgaggtgaat | 1020 |
| ctggatcggt accagacagc cctggaggag gtgctgagct ggctgctgag tgctgaagac | 1080 |
| acactgcagg cccagggcga aatttccaat gacgtggaag tggtgaagga tcagttccac | 1140 |
| acacacgagg gctatatgat ggacctgaca gctcaccagg ggcgcgtggg caatatcctg | 1200 |
| cagctgggct ctaaactgat cggcaccggg aaactgagtg aggacgagga aacagaagtg | 1260 |
| caggagcaga tgaacctgct gaacagccgc tgggagtgtc tgagagtggc tagtatggag | 1320 |
| aagcagtcca acctgcaccg ggtgctgatg gacctgcaga accagaaact gaaagagctg | 1380 |
| aacgactggc tgacaaagac tgaggaacgc acaaggaaga tggaggagga gccactggga | 1440 |
| cccgacctgg aggatctgaa gagacaggtg cagcagcata aggtgctgca ggaggatctg | 1500 |
| gaacaggagc aggtgcgggt gaactccctg acacatatgg tggtggtggt ggacgaatct | 1560 |
| agtggagatc acgccaccgc cgccctggag gaacagctga aggtgctggg ggaccggtgg | 1620 |
| gccaacattt gccggtggac cgaggacagg tgggtgctgc tgcaggacat cctgctgaaa | 1680 |
| tggcagaggc tgaccgagga gcagtgtctg tttagtgctt ggctgagcga gaaagaggac | 1740 |
| gccgtgaaca agatccacac aaccggcttt aaggatcaga acgaaatgct gtctagcctg | 1800 |
| cagaaactgg ctgtgctgaa ggccgatctg gagaaaaaga agcagagcat gggcaaactg | 1860 |
| tatagcctga aacaggacct gctgagcacc ctgaagaaca agagcgtgac ccagaagaca | 1920 |
| gaagcctggc tggataactt tgcccgctgc tgggacaacc tggtgcagaa actggagaaa | 1980 |
| agtacagctc agatctctca ggctgtgacc acaacccagc ctagcctgac ccagacaacc | 2040 |
| gtgatggaaa ccgtgaccac cgtgacaacc cgcgaacaga tcctggtgaa acatgcccag | 2100 |
| gaagagctgc cacctccacc tccccagaag aagagaaccc tggagcggct gcaggagctg | 2160 |
| caggaagcca ctgacgaact ggacctgaag ctgaggcagg ccgaagtgat taaggggtct | 2220 |
| tggcagcctg tgggcgatct gctgattgat tccctgcagg accacctgga aaaggtgaag | 2280 |
| gctctgagag gcgaaattgc tccactgaag gagaacgtga gtcatgtgaa cgatctggct | 2340 |
| agacagctga caacactggg catccagctg agcccataca atctgagcac actggaggac | 2400 |
| ctgaatacca ggtggaagct gctgcaggtg gctgtggaag accgggtgcg gcagctgcat | 2460 |
| gaggcccatc gcgacttcgg accagccagc cagcactttc tgagcacatc cgtgcagggg | 2520 |
| ccctgggaga gggccatttc tcccaacaag gtgccctact atattaatca cgagacccag | 2580 |
| accacttgtt gggaccatcc caagatgaca gaactgtacc agtccctggc cgatctgaac | 2640 |
| aacgtgaggt ttagcgctta cagaaccgct atgaagctga gacggctgca gaaggccctg | 2700 |
| tgcctggatc tgctgtccct gtccgccgcc tgcgatgccc tggatcagca taatctgaag | 2760 |
| cagaacgatc agccaatgga tatcctgcag atcatcaact gcctgaccac tatctacgac | 2820 |
| aggctggagc aggagcacaa caacctggtg aacgtgcctc tgtgcgtgga tatgtgcctg | 2880 |
| aactggctgc tgaacgtgta tgacactggg cgcaccggcc ggatcagagt gctgagtttt | 2940 |
| aaaactggga ttatctccct gtgtaaggcc cacctggagg acaagtacag gtacctgttc | 3000 |
| aagcaggtgg ctagtagcac tggattttgt gaccagcgcc gcctgggact gctgctgcat | 3060 |
| gatagtatcc agattcctag acagctggga gaggtggcta gtttcggagg atctaacatc | 3120 |
| gaacccagcg tgcgcagctg tttccagttt gccaataaca aacctgaaat cgaggctgct | 3180 |
| ctgttcctgg attggatgcg cctggaacca cagagcatgg tgtggctgcc tgtgctgcac | 3240 |
| agagtggctg ccgccgaaac tgccaagcac caggctaaat gcaacatctg caaggaatgt | 3300 |
| cccattatcg gctttcgcta caggagtctg aaacatttta actacgatat ttgccagagc | 3360 |
| tgcttctttt ccggaagagt ggccaaagga cacaagatgc actaccctat ggtggaatat | 3420 |
| tgcaccccaa ctacatctgg cgaagatgtg cgcgattttg ccaaggtgct gaagaataag | 3480 |
| tttcggacta agaggtactt cgccaagcac ccccgcatgg ggtatctgcc agtgcagaca | 3540 |
| gtgctggaag gagacaatat ggagaccgat acaatgtga | 3579 |
| <210> SEQ ID NO: 2 | |
| <211> 810 | |
| <212> DNA | |
| <213> Adeno-associated virus | |
| <400> 2 | |
| gtttaaacaa gcttgcatgt ctaagctaga cccttcagat taaaaataac tgaggtaagg | 60 |
| gcctgggtag gggaggtggt gtgagacgct cctgtctctc ctctatctgc ccatcggccc | 120 |
| tttggggagg aggaatgtgc ccaaggacta aaaaaaggcc atggagccag aggggcgagg | 180 |
| gcaacagacc tttcatgggc aaaccttggg gccctgctgt ctagcatgcc ccactacggg | 240 |
| tctaggctgc ccatgtaagg aggcaaggcc tggggacacc cgagatgcct ggttataatt | 300 |
| aacccagaca tgtggctgcc cccccccccc caacacctgc tgcctctaaa aataaccctg | 360 |
| tccctggtgg atcccctgca tgcgaagatc ttcgaacaag gctgtggggg actgagggca | 420 |
| ggctgtaaca ggcttggggg ccagggctta tacgtgcctg ggactcccaa agtattactg | 480 |
| ttccatgttc ccggcgaagg gccagctgtc ccccgccagc tagactcagc acttagttta | 540 |
| ggaaccagtg agcaagtcag cccttggggc agcccataca aggccatggg gctgggcaag | 600 |
| ctgcacgcct gggtccgggg tgggcacggt gcccgggcaa cgagctgaaa gctcatctgc | 660 |
| tctcaggggc ccctccctgg ggacagcccc tcctggctag tcacaccctg taggctcctc | 720 |
| tatataaccc aggggcacag gggctgccct cattctacca ccacctccac agcacagaca | 780 |
| gacactcagg agccagccag cggcgcgccc | 810 |
| <210> SEQ ID NO: 3 | |
| <211> 8562 | |
| <212> DNA | |
| <213> Adeno-associated virus | |
| <400> 3 | |
| gcccaatacg caaaccgcct ctccccgcgc gttggccgat tcattaatgc agctgcgcgc | 60 |
| tcgctcgctc actgaggccg cccgggcaaa gcccgggcgt cgggcgacct ttggtcgccc | 120 |
| ggcctcagtg agcgagcgag cgcgcagaga gggagtggcc aactccatca ctaggggttc | 180 |
| cttgtagtta atgattaacc cgccatgcta cttatctacg tagccatgct ctagagttta | 240 |
| aacaagcttg catgtctaag ctagaccctt cagattaaaa ataactgagg taagggcctg | 300 |
| ggtaggggag gtggtgtgag acgctcctgt ctctcctcta tctgcccatc ggccctttgg | 360 |
| ggaggaggaa tgtgcccaag gactaaaaaa aggccatgga gccagagggg cgagggcaac | 420 |
| agacctttca tgggcaaacc ttggggccct gctgtctagc atgccccact acgggtctag | 480 |
| gctgcccatg taaggaggca aggcctgggg acacccgaga tgcctggtta taattaaccc | 540 |
| agacatgtgg ctgccccccc ccccccaaca cctgctgcct ctaaaaataa ccctgtccct | 600 |
| ggtggatccc ctgcatgcga agatcttcga acaaggctgt gggggactga gggcaggctg | 660 |
| taacaggctt gggggccagg gcttatacgt gcctgggact cccaaagtat tactgttcca | 720 |
| tgttcccggc gaagggccag ctgtcccccg ccagctagac tcagcactta gtttaggaac | 780 |
| cagtgagcaa gtcagccctt ggggcagccc atacaaggcc atggggctgg gcaagctgca | 840 |
| cgcctgggtc cggggtgggc acggtgcccg ggcaacgagc tgaaagctca tctgctctca | 900 |
| ggggcccctc cctggggaca gcccctcctg gctagtcaca ccctgtaggc tcctctatat | 960 |
| aacccagggg cacaggggct gccctcattc taccaccacc tccacagcac agacagacac | 1020 |
| tcaggagcag ccagcggcgc gcccaggtaa gtttagtctt tttgtctttt atttcaggtc | 1080 |
| ccggatccgg tggtggtgca aatcaaagaa ctgctcctca gtggatgttg cctttacttc | 1140 |
| taggcctgta cggaagtgtt acttctgctc taaaagctgc ggaattgtac ccgcggccgc | 1200 |
| caccatgctg tggtgggagg aggtggagga ttgttatgaa agggaggacg tgcagaagaa | 1260 |
| gacttttacc aagtgggtga acgctcagtt cagcaaattt gggaagcagc acatcgagaa | 1320 |
| tctgttttcc gacctgcagg atgggagacg gctgctggat ctgctggaag gactgactgg | 1380 |
| ccagaagctg cccaaagaga aggggagcac tagggtgcac gccctgaaca acgtgaacaa | 1440 |
| agctctgaga gtgctgcaga acaacaacgt ggatctggtg aatattggca gtactgatat | 1500 |
| cgtggacggg aaccacaaac tgacactggg cctgatctgg aacattattc tgcactggca | 1560 |
| ggtgaaaaat gtgatgaaga acatcatggc cgggctgcag cagaccaatt ccgagaagat | 1620 |
| cctgctgtct tgggtgcggc agagcacccg caactatccc caggtgaacg tgattaactt | 1680 |
| cactacatcc tggagcgacg ggctggccct gaatgctctg attcacagcc acaggcctga | 1740 |
| tctgttcgac tggaatagcg tggtgtgcca gcagtctgcc acacagcgcc tggaacatgc | 1800 |
| cttcaatatc gctcggtacc agctggggat cgaaaaactg ctggacccag aggatgtgga | 1860 |
| cactacatac ccagataaaa agtctattct gatgtacatt actagcctgt tccaggtgct | 1920 |
| gccacagcag gtgtctattg aagccattca ggaggtggaa atgctgcccc gcccccccaa | 1980 |
| agtgactaaa gaggagcatt ttcagctgca tcatcagatg cattacagcc agcagattac | 2040 |
| cgtgagcctg gctcagggat atgagcgcac cagtagtcca aaaccacggt tcaagtccta | 2100 |
| cgcttatacc caggctgcct acgtgacaac tagcgaccct actagatccc cctttccatc | 2160 |
| ccagcacctg gaggccccag aggacaagag ctttgggtcc agcctgatgg aaagcgaggt | 2220 |
| gaatctggat cggtaccaga cagccctgga ggaggtgctg agctggctgc tgagtgctga | 2280 |
| agacacactg caggcccagg gcgaaatttc caatgacgtg gaagtggtga aggatcagtt | 2340 |
| ccacacacac gagggctata tgatggacct gacagctcac caggggcgcg tgggcaatat | 2400 |
| cctgcagctg ggctctaaac tgatcggcac cgggaaactg agtgaggacg aggaaacaga | 2460 |
| agtgcaggag cagatgaacc tgctgaacag ccgctgggag tgtctgagag tggctagtat | 2520 |
| ggagaagcag tccaacctgc accgggtgct gatggacctg cagaaccaga aactgaaaga | 2580 |
| gctgaacgac tggctgacaa agactgagga acgcacaagg aagatggagg aggagccact | 2640 |
| gggacccgac ctggaggatc tgaagagaca ggtgcagcag cataaggtgc tgcaggagga | 2700 |
| tctggaacag gagcaggtgc gggtgaactc cctgacacat atggtggtgg tggtggacga | 2760 |
| atctagtgga gatcacgcca ccgccgccct ggaggaacag ctgaaggtgc tgggggaccg | 2820 |
| gtgggccaac atttgccggt ggaccgagga caggtgggtg ctgctgcagg acatcctgct | 2880 |
| gaaatggcag aggctgaccg aggagcagtg tctgtttagt gcttggctga gcgagaaaga | 2940 |
| ggacgccgtg aacaagatcc acacaaccgg ctttaaggat cagaacgaaa tgctgtctag | 3000 |
| cctgcagaaa ctggctgtgc tgaaggccga tctggagaaa aagaagcaga gcatgggcaa | 3060 |
| actgtatagc ctgaaacagg acctgctgag caccctgaag aacaagagcg tgacccagaa | 3120 |
| gacagaagcc tggctggata actttgcccg ctgctgggac aacctggtgc agaaactgga | 3180 |
| gaaaagtaca gctcagatct ctcaggctgt gaccacaacc cagcctagcc tgacccagac | 3240 |
| aaccgtgatg gaaaccgtga ccaccgtgac aacccgcgaa cagatcctgg tgaaacatgc | 3300 |
| ccaggaagag ctgccacctc cacctcccca gaagaagaga accctggagc ggctgcagga | 3360 |
| gctgcaggaa gccactgacg aactggacct gaagctgagg caggccgaag tgattaaggg | 3420 |
| gtcttggcag cctgtgggcg atctgctgat tgattccctg caggaccacc tggaaaaggt | 3480 |
| gaaggctctg agaggcgaaa ttgctccact gaaggagaac gtgagtcatg tgaacgatct | 3540 |
| ggctagacag ctgacaacac tgggcatcca gctgagccca tacaatctga gcacactgga | 3600 |
| ggacctgaat accaggtgga agctgctgca ggtggctgtg gaagaccggg tgcggcagct | 3660 |
| gcatgaggcc catcgcgact tcggaccagc cagccagcac tttctgagca catccgtgca | 3720 |
| ggggccctgg gagagggcca tttctcccaa caaggtgccc tactatatta atcacgagac | 3780 |
| ccagaccact tgttgggacc atcccaagat gacagaactg taccagtccc tggccgatct | 3840 |
| gaacaacgtg aggtttagcg cttacagaac cgctatgaag ctgagacggc tgcagaaggc | 3900 |
| cctgtgcctg gatctgctgt ccctgtccgc cgcctgcgat gccctggatc agcataatct | 3960 |
| gaagcagaac gatcagccaa tggatatcct gcagatcatc aactgcctga ccactatcta | 4020 |
| cgacaggctg gagcaggagc acaacaacct ggtgaacgtg cctctgtgcg tggatatgtg | 4080 |
| cctgaactgg ctgctgaacg tgtatgacac tgggcgcacc ggccggatca gagtgctgag | 4140 |
| ttttaaaact gggattatct ccctgtgtaa ggcccacctg gaggacaagt acaggtacct | 4200 |
| gttcaagcag gtggctagta gcactggatt ttgtgaccag cgccgcctgg gactgctgct | 4260 |
| gcatgatagt atccagattc ctagacagct gggagaggtg gctagtttcg gaggatctaa | 4320 |
| catcgaaccc agcgtgcgca gctgtttcca gtttgccaat aacaaacctg aaatcgaggc | 4380 |
| tgctctgttc ctggattgga tgcgcctgga accacagagc atggtgtggc tgcctgtgct | 4440 |
| gcacagagtg gctgccgccg aaactgccaa gcaccaggct aaatgcaaca tctgcaagga | 4500 |
| atgtcccatt atcggctttc gctacaggag tctgaaacat tttaactacg atatttgcca | 4560 |
| gagctgcttc ttttccggaa gagtggccaa aggacacaag atgcactacc ctatggtgga | 4620 |
| atattgcacc ccaactacat ctggcgaaga tgtgcgcgat tttgccaagg tgctgaagaa | 4680 |
| taagtttcgg actaagaggt acttcgccaa gcacccccgc atggggtatc tgccagtgca | 4740 |
| gacagtgctg gaaggagaca atatggagac cgatacaatg tgagcggccg caataaaaga | 4800 |
| tctttatttt cattagatct gtgtgttggt tttttgtgtg tctagagcat ggctacgtag | 4860 |
| ataagtagca tggcgggtta atcattaact acaaggaacc cctagtgatg gagttggcca | 4920 |
| ctccctctct gcgcgctcgc tcgctcactg aggccgggcg accaaaggtc gcccgacgcc | 4980 |
| cgggctttgc ccgggcggcc tcagtgagcg agcgagcgcg cagctggcgt aatagcgaag | 5040 |
| aggcccgcac cgatcgccct tcccaacagt tgcgcagcct gaatggcgaa tggcgattcc | 5100 |
| gttgcaatgg ctggcggtaa tattgttctg gatattacca gcaaggccga tagtttgagt | 5160 |
| tcttctactc aggcaagtga tgttattact aatcaaagaa gtattgcgac aacggttaat | 5220 |
| ttgcgtgatg gacagactct tttactcggt ggcctcactg attataaaaa cacttctcag | 5280 |
| gattctggcg taccgttcct gtctaaaatc cctttaatcg gcctcctgtt tagctcccgc | 5340 |
| tctgattcta acgaggaaag cacgttatac gtgctcgtca aagcaaccat agtacgcgcc | 5400 |
| ctgtagcggc gcattaagcg cggcgggtgt ggtggttacg cgcagcgtga ccgctacact | 5460 |
| tgccagcgcc ctagcgcccg ctcctttcgc tttcttccct tcctttctcg ccacgttcgc | 5520 |
| cggctttccc cgtcaagctc taaatcgggg gctcccttta gggttccgat ttagtgcttt | 5580 |
| acggcacctc gaccccaaaa aacttgatta gggtgatggt tcacgtagtg ggccatcgcc | 5640 |
| ctgatagacg gtttttcgcc ctttgacgtt ggagtccacg ttctttaata gtggactctt | 5700 |
| gttccaaact ggaacaacac tcaaccctat ctcggtctat tcttttgatt tataagggat | 5760 |
| tttgccgatt tcggcctatt ggttaaaaaa tgagctgatt taacaaaaat ttaacgcgaa | 5820 |
| ttttaacaaa atattaacgc ttacaattta aatatttgct tatacaatct tcctgttttt | 5880 |
| ggggcttttc tgattatcaa ccggggtaca tatgattgac atgctagttt tacgattacc | 5940 |
| gttcatcgat tctcttgttt gctccagact ctcaggcaat gacctgatag cctttgtaga | 6000 |
| gacctctcaa aaatagctac cctctccggc atgaatttat cagctagaac ggttgaatat | 6060 |
| catattgatg gtgatttgac tgtctccggc ctttctcacc cgtttgaatc tttacctaca | 6120 |
| cattactcag gcattgcatt taaaatatat gagggttcta aaaattttta tccttgcgtt | 6180 |
| gaaataaagg cttctcccgc aaaagtatta cagggtcata atgtttttgg tacaaccgat | 6240 |
| ttagctttat gctctgaggc tttattgctt aattttgcta attctttgcc ttgcctgtat | 6300 |
| gatttattgg atgttggaat cgcctgatgc ggtattttct ccttacgcat ctgtgcggta | 6360 |
| tttcacaccg catatggtgc actctcagta caatctgctc tgatgccgca tagttaagcc | 6420 |
| agccccgaca cccgccaaca cccgctgacg cgccctgacg ggcttgtctg ctcccggcat | 6480 |
| ccgcttacag acaagctgtg accgtctccg ggagctgcat gtgtcagagg ttttcaccgt | 6540 |
| catcaccgaa acgcgcgaga cgaaagggcc tcgtgatacg cctattttta taggttaatg | 6600 |
| tcatgataat aatggtttct tagacgtcag gtggcacttt tcggggaaat gtgcgcggaa | 6660 |
| cccctatttg tttatttttc taaatacatt caaatatgta tccgctcatg agacaataac | 6720 |
| cctgataaat gcttcaataa tattgaaaaa ggaagagtat gagtattcaa catttccgtg | 6780 |
| tcgcccttat tccctttttt gcggcatttt gccttcctgt ttttgctcac ccagaaacgc | 6840 |
| tggtgaaagt aaaagatgct gaagatcagt tgggtgcacg agtgggttac atcgaactgg | 6900 |
| atctcaacag cggtaagatc cttgagagtt ttcgccccga agaacgtttt ccaatgatga | 6960 |
| gcacttttaa agttctgcta tgtggcgcgg tattatcccg tattgacgcc gggcaagagc | 7020 |
| aactcggtcg ccgcatacac tattctcaga atgacttggt tgagtactca ccagtcacag | 7080 |
| aaaagcatct tacggatggc atgacagtaa gagaattatg cagtgctgcc ataaccatga | 7140 |
| gtgataacac tgcggccaac ttacttctga caacgatcgg aggaccgaag gagctaaccg | 7200 |
| cttttttgca caacatgggg gatcatgtaa ctcgccttga tcgttgggaa ccggagctga | 7260 |
| atgaagccat accaaacgac gagcgtgaca ccacgatgcc tgtagcaatg gcaacaacgt | 7320 |
| tgcgcaaact attaactggc gaactactta ctctagcttc ccggcaacaa ttaatagact | 7380 |
| ggatggaggc ggataaagtt gcaggaccac ttctgcgctc ggcccttccg gctggctggt | 7440 |
| ttattgctga taaatctgga gccggtgagc gtgggtctcg cggtatcatt gcagcactgg | 7500 |
| ggccagatgg taagccctcc cgtatcgtag ttatctacac gacggggagt caggcaacta | 7560 |
| tggatgaacg aaatagacag atcgctgaga taggtgcctc actgattaag cattggtaac | 7620 |
| tgtcagacca agtttactca tatatacttt agattgattt aaaacttcat ttttaattta | 7680 |
| aaaggatcta ggtgaagatc ctttttgata atctcatgac caaaatccct taacgtgagt | 7740 |
| tttcgttcca ctgagcgtca gaccccgtag aaaagatcaa aggatcttct tgagatcctt | 7800 |
| tttttctgcg cgtaatctgc tgcttgcaaa caaaaaaacc accgctacca gcggtggttt | 7860 |
| gtttgccgga tcaagagcta ccaactcttt ttccgaaggt aactggcttc agcagagcgc | 7920 |
| agataccaaa tactgttctt ctagtgtagc cgtagttagg ccaccacttc aagaactctg | 7980 |
| tagcaccgcc tacatacctc gctctgctaa tcctgttacc agtggctgct gccagtggcg | 8040 |
| ataagtcgtg tcttaccggg ttggactcaa gacgatagtt accggataag gcgcagcggt | 8100 |
| cgggctgaac ggggggttcg tgcacacagc ccagcttgga gcgaacgacc tacaccgaac | 8160 |
| tgagatacct acagcgtgag ctatgagaaa gcgccacgct tcccgaaggg agaaaggcgg | 8220 |
| acaggtatcc ggtaagcggc agggtcggaa caggagagcg cacgagggag cttccagggg | 8280 |
| gaaacgcctg gtatctttat agtcctgtcg ggtttcgcca cctctgactt gagcgtcgat | 8340 |
| ttttgtgatg ctcgtcaggg gggcggagcc tatggaaaaa cgccagcaac gcggcctttt | 8400 |
| tacggttcct ggccttttgc tggccttttg ctcacatgtt ctttcctgcg ttatcccctg | 8460 |
| attctgtgga taaccgtatt accgcctttg agtgagctga taccgctcgc cgcagccgaa | 8520 |
| cgaccgagcg cagcgagtca gtgagcgagg aagcggaaga gc | 8562 |
| <210> SEQ ID NO: 4 | |
| <211> 564 | |
| <212> DNA | |
| <213> Adeno-associated virus | |
| <400> 4 | |
| cagccactat gggtctaggc tgcccatgta aggaggcaag gcctggggac acccgagatg | 60 |
| cctggttata attaacccag acatgtggct gctccccccc cccaacacct gctgcctgag | 120 |
| cctcaccccc accccggtgc ctgggtctta ggctctgtac accatggagg agaagctcgc | 180 |
| tctaaaaata accctgtccc tggtgggctg tgggggactg agggcaggct gtaacaggct | 240 |
| tgggggccag ggcttatacg tgcctgggac tcccaaagta ttactgttcc atgttcccgg | 300 |
| cgaagggcca gctgtccccc gccagctaga ctcagcactt agtttaggaa ccagtgagca | 360 |
| agtcagccct tggggcagcc catacaaggc catggggctg ggcaagctgc acgcctgggt | 420 |
| ccggggtggg cacggtgccc gggcaacgag ctgaaagctc atctgctctc aggggcccct | 480 |
| ccctggggac agcccctcct ggctagtcac accctgtagg ctcctctata taacccaggg | 540 |
| gcacaggggc tgcccccggg tcac | 564 |
| <210> SEQ ID NO: 5 | |
| <211> 8409 | |
| <212> DNA | |
| <213> Adeno-associated virus | |
| <400> 5 | |
| gcccaatacg caaaccgcct ctccccgcgc gttggccgat tcattaatgc agctggcgcg | 60 |
| ctcgctcgct cactgaggcc gcccgggcaa agcccgggcg tcgggcgacc tttggtcgcc | 120 |
| cggcctcagt gagcgagcga gcgcgcagag agggagtggc caactccatc actaggggtt | 180 |
| ccttgtagtt aatgattaac ccgccatgct aattatctac gtagccatgt ctagacagcc | 240 |
| actatgggtc taggctgccc atgtaaggag gcaaggcctg gggacacccg agatgcctgg | 300 |
| ttataattaa cccagacatg tggctgctcc ccccccccaa cacctgctgc ctgagcctca | 360 |
| cccccacccc ggtgcctggg tcttaggctc tgtacaccat ggaggagaag ctcgctctaa | 420 |
| aaataaccct gtccctggtg ggctgtgggg gactgagggc aggctgtaac aggcttgggg | 480 |
| gccagggctt atacgtgcct gggactccca aagtattact gttccatgtt cccggcgaag | 540 |
| ggccagctgt cccccgccag ctagactcag cacttagttt aggaaccagt gagcaagtca | 600 |
| gcccttgggg cagcccatac aaggccatgg ggctgggcaa gctgcacgcc tgggtccggg | 660 |
| gtgggcacgg tgcccgggca acgagctgaa agctcatctg ctctcagggg cccctccctg | 720 |
| gggacagccc ctcctggcta gtcacaccct gtaggctcct ctatataacc caggggcaca | 780 |
| ggggctgccc ccgggtcacc accacctcca cagcacagac agacactcag gagccagcca | 840 |
| gccaggtaag tttagtcttt ttgtctttta tttcaggtcc cggatccggt ggtggtgcaa | 900 |
| atcaaagaac tgctcctcag tggatgttgc ctttacttct aggcctgtac ggaagtgtta | 960 |
| cttctgctct aaaagctgcg gaattgtacc cgcggccgcc accatgctgt ggtgggagga | 1020 |
| ggtggaggat tgttatgaaa gggaggacgt gcagaagaag acttttacca agtgggtgaa | 1080 |
| cgctcagttc agcaaatttg ggaagcagca catcgagaat ctgttttccg acctgcagga | 1140 |
| tgggagacgg ctgctggatc tgctggaagg actgactggc cagaagctgc ccaaagagaa | 1200 |
| ggggagcact agggtgcacg ccctgaacaa cgtgaacaaa gctctgagag tgctgcagaa | 1260 |
| caacaacgtg gatctggtga atattggcag tactgatatc gtggacggga accacaaact | 1320 |
| gacactgggc ctgatctgga acattattct gcactggcag gtgaaaaatg tgatgaagaa | 1380 |
| catcatggcc gggctgcagc agaccaattc cgagaagatc ctgctgtctt gggtgcggca | 1440 |
| gagcacccgc aactatcccc aggtgaacgt gattaacttc actacatcct ggagcgacgg | 1500 |
| gctggccctg aatgctctga ttcacagcca caggcctgat ctgttcgact ggaatagcgt | 1560 |
| ggtgtgccag cagtctgcca cacagcgcct ggaacatgcc ttcaatatcg ctcggtacca | 1620 |
| gctggggatc gaaaaactgc tggacccaga ggatgtggac actacatacc cagataaaaa | 1680 |
| gtctattctg atgtacatta ctagcctgtt ccaggtgctg ccacagcagg tgtctattga | 1740 |
| agccattcag gaggtggaaa tgctgccccg cccccccaaa gtgactaaag aggagcattt | 1800 |
| tcagctgcat catcagatgc attacagcca gcagattacc gtgagcctgg ctcagggata | 1860 |
| tgagcgcacc agtagtccaa aaccacggtt caagtcctac gcttataccc aggctgccta | 1920 |
| cgtgacaact agcgacccta ctagatcccc ctttccatcc cagcacctgg aggccccaga | 1980 |
| ggacaagagc tttgggtcca gcctgatgga aagcgaggtg aatctggatc ggtaccagac | 2040 |
| agccctggag gaggtgctga gctggctgct gagtgctgaa gacacactgc aggcccaggg | 2100 |
| cgaaatttcc aatgacgtgg aagtggtgaa ggatcagttc cacacacacg agggctatat | 2160 |
| gatggacctg acagctcacc aggggcgcgt gggcaatatc ctgcagctgg gctctaaact | 2220 |
| gatcggcacc gggaaactga gtgaggacga ggaaacagaa gtgcaggagc agatgaacct | 2280 |
| gctgaacagc cgctgggagt gtctgagagt ggctagtatg gagaagcagt ccaacctgca | 2340 |
| ccgggtgctg atggacctgc agaaccagaa actgaaagag ctgaacgact ggctgacaaa | 2400 |
| gactgaggaa cgcacaagga agatggagga ggagccactg ggacccgacc tggaggatct | 2460 |
| gaagagacag gtgcagcagc ataaggtgct gcaggaggat ctggaacagg agcaggtgcg | 2520 |
| ggtgaactcc ctgacacata tggtggtggt ggtggacgaa tctagtggag atcacgccac | 2580 |
| cgccgccctg gaggaacagc tgaaggtgct gggggaccgg tgggccaaca tttgccggtg | 2640 |
| gaccgaggac aggtgggtgc tgctgcagga catcctgctg aaatggcaga ggctgaccga | 2700 |
| ggagcagtgt ctgtttagtg cttggctgag cgagaaagag gacgccgtga acaagatcca | 2760 |
| cacaaccggc tttaaggatc agaacgaaat gctgtctagc ctgcagaaac tggctgtgct | 2820 |
| gaaggccgat ctggagaaaa agaagcagag catgggcaaa ctgtatagcc tgaaacagga | 2880 |
| cctgctgagc accctgaaga acaagagcgt gacccagaag acagaagcct ggctggataa | 2940 |
| ctttgcccgc tgctgggaca acctggtgca gaaactggag aaaagtacag ctcagatctc | 3000 |
| tcaggctgtg accacaaccc agcctagcct gacccagaca accgtgatgg aaaccgtgac | 3060 |
| caccgtgaca acccgcgaac agatcctggt gaaacatgcc caggaagagc tgccacctcc | 3120 |
| acctccccag aagaagagaa ccctggagcg gctgcaggag ctgcaggaag ccactgacga | 3180 |
| actggacctg aagctgaggc aggccgaagt gattaagggg tcttggcagc ctgtgggcga | 3240 |
| tctgctgatt gattccctgc aggaccacct ggaaaaggtg aaggctctga gaggcgaaat | 3300 |
| tgctccactg aaggagaacg tgagtcatgt gaacgatctg gctagacagc tgacaacact | 3360 |
| gggcatccag ctgagcccat acaatctgag cacactggag gacctgaata ccaggtggaa | 3420 |
| gctgctgcag gtggctgtgg aagaccgggt gcggcagctg catgaggccc atcgcgactt | 3480 |
| cggaccagcc agccagcact ttctgagcac atccgtgcag gggccctggg agagggccat | 3540 |
| ttctcccaac aaggtgccct actatattaa tcacgagacc cagaccactt gttgggacca | 3600 |
| tcccaagatg acagaactgt accagtccct ggccgatctg aacaacgtga ggtttagcgc | 3660 |
| ttacagaacc gctatgaagc tgagacggct gcagaaggcc ctgtgcctgg atctgctgtc | 3720 |
| cctgtccgcc gcctgcgatg ccctggatca gcataatctg aagcagaacg atcagccaat | 3780 |
| ggatatcctg cagatcatca actgcctgac cactatctac gacaggctgg agcaggagca | 3840 |
| caacaacctg gtgaacgtgc ctctgtgcgt ggatatgtgc ctgaactggc tgctgaacgt | 3900 |
| gtatgacact gggcgcaccg gccggatcag agtgctgagt tttaaaactg ggattatctc | 3960 |
| cctgtgtaag gcccacctgg aggacaagta caggtacctg ttcaagcagg tggctagtag | 4020 |
| cactggattt tgtgaccagc gccgcctggg actgctgctg catgatagta tccagattcc | 4080 |
| tagacagctg ggagaggtgg ctagtttcgg aggatctaac atcgaaccca gcgtgcgcag | 4140 |
| ctgtttccag tttgccaata acaaacctga aatcgaggct gctctgttcc tggattggat | 4200 |
| gcgcctggaa ccacagagca tggtgtggct gcctgtgctg cacagagtgg ctgccgccga | 4260 |
| aactgccaag caccaggcta aatgcaacat ctgcaaggaa tgtcccatta tcggctttcg | 4320 |
| ctacaggagt ctgaaacatt ttaactacga tatttgccag agctgcttct tttccggaag | 4380 |
| agtggccaaa ggacacaaga tgcactaccc tatggtggaa tattgcaccc caactacatc | 4440 |
| tggcgaagat gtgcgcgatt ttgccaaggt gctgaagaat aagtttcgga ctaagaggta | 4500 |
| cttcgccaag cacccccgca tggggtatct gccagtgcag acagtgctgg aaggagacaa | 4560 |
| tatggagacc gatacaatgt gagcggccgc aataaaagat ctttattttc attagatctg | 4620 |
| tgtgttggtt ttttgtgtgt ctagagcatg gctacgtaga taagtagcat ggcgggttaa | 4680 |
| tcattaacta caaggaaccc ctagtgatgg agttggccac tccctctctg cgcgctcgct | 4740 |
| cgctcactga ggccgggcga ccaaaggtcg cccgacgccc gggctttgcc cgggcggcct | 4800 |
| cagtgagcga gcgagcgcgc cagctggcgt aatagcgaag aggcccgcac cgatcgccct | 4860 |
| tcccaacagt tgcgcagcct gaatggcgaa tggaagttcc agacgattga gcgtcaaaat | 4920 |
| gtaggtattt ccatgagcgt ttttcctgtt gcaatggctg gcggtaatat tgttctggat | 4980 |
| attaccagca aggccgatag tttgagttct tctactcagg caagtgatgt tattactaat | 5040 |
| caaagaagta ttgcgacaac ggttaatttg cgtgatggac agactctttt actcggtggc | 5100 |
| ctcactgatt ataaaaacac ttctcaggat tctggcgtac cgttcctgtc taaaatccct | 5160 |
| ttaatcggcc tcctgtttag ctcccgctct gattctaacg aggaaagcac gttatacgtg | 5220 |
| ctcgtcaaag caaccatagt acgcgccctg tagcggcgca ttaagcgcgg cgggtgtggt | 5280 |
| ggttacgcgc agcgtgaccg ctacacttgc cagcgcccta gcgcccgctc ctttcgcttt | 5340 |
| cttcccttcc tttctcgcca cgttcgccgg ctttccccgt caagctctaa atcgggggct | 5400 |
| ccctttaggg ttccgattta gtgatttacg gcacctcgac cccaaaaaac ttgattaggg | 5460 |
| tgatggttca cgtagtgggc catcgccctg atagacggtt tttcgccctt tgacgttgga | 5520 |
| gtccacgttc tttaatagtg gactcttgtt ccaaactgga acaacactca accctatctc | 5580 |
| ggtctattct tttgatttat aagggatttt gccgatttcg gcctattggt taaaaaatga | 5640 |
| gctgatttaa caaaaattta acgcgaattt taacaaaata ttaacgttta caatttaaat | 5700 |
| atttgcttat acaatcttcc tgtttttggg gcttttctga ttatcaaccg gggtacatat | 5760 |
| gattgacatg ctagttttac gattaccgtt catcgattct cttgtttgct ccagactctc | 5820 |
| aggcaatgac ctgatagcct ttgtagagac ctctcaaaaa tagctaccct ctccggcatg | 5880 |
| aatttatcag ctagaacggt tgaatatcat attgatggtg atttgactgt ctccggcctt | 5940 |
| tctcacccgt ttgaatcttt acctacacat tactcaggca ttgcatttaa aatatatgag | 6000 |
| ggttctaaaa atttttatcc ttgcgttgaa ataaaggctt ctcccgcaaa agtattacag | 6060 |
| ggtcataatg tttttggtac aaccgattta gctttatgct ctgaggcttt attgcttaat | 6120 |
| tttgctaatt ctttgccttg cctgtatgat ttattggatg ttggaagttc ctgatgcggt | 6180 |
| attttctcct tacgcatctg tgcggtattt cacaccgcat atggtgcact ctcagtacaa | 6240 |
| tctgctctga tgccgcatag ttaagccagc cccgacaccc gccaacaccc gctgacgcgc | 6300 |
| cctgacgggc ttgtctgctc ccggcatccg cttacagaca agctgtgacc gtctccggga | 6360 |
| gctgcatgtg tcagaggttt tcaccgtcat caccgaaacg cgcgagacga aagggcctcg | 6420 |
| tgatacgcct atttttatag gttaatgtca tgataataat ggtttcttag acgtcaggtg | 6480 |
| gcacttttcg gggaaatgtg cgcggaaccc ctatttgttt atttttctaa atacattcaa | 6540 |
| atatgtatcc gctcatgaga caataaccct gataaatgct tcaataatat tgaaaaagga | 6600 |
| agagtatgag tattcaacat ttccgtgtcg cccttattcc cttttttgcg gcattttgcc | 6660 |
| ttcctgtttt tgctcaccca gaaacgctgg tgaaagtaaa agatgctgaa gatcagttgg | 6720 |
| gtgcacgagt gggttacatc gaactggatc tcaacagcgg taagatcctt gagagttttc | 6780 |
| gccccgaaga acgttttcca atgatgagca cttttaaagt tctgctatgt ggcgcggtat | 6840 |
| tatcccgtat tgacgccggg caagagcaac tcggtcgccg catacactat tctcagaatg | 6900 |
| acttggttga gtactcacca gtcacagaaa agcatcttac ggatggcatg acagtaagag | 6960 |
| aattatgcag tgctgccata accatgagtg ataacactgc ggccaactta cttctgacaa | 7020 |
| cgatcggagg accgaaggag ctaaccgctt ttttgcacaa catgggggat catgtaactc | 7080 |
| gccttgatcg ttgggaaccg gagctgaatg aagccatacc aaacgacgag cgtgacacca | 7140 |
| cgatgcctgt agcaatggca acaacgttgc gcaaactatt aactggcgaa ctacttactc | 7200 |
| tagcttcccg gcaacaatta atagactgga tggaggcgga taaagttgca ggaccacttc | 7260 |
| tgcgctcggc ccttccggct ggctggttta ttgctgataa atctggagcc ggtgagcgtg | 7320 |
| ggtctcgcgg tatcattgca gcactggggc cagatggtaa gccctcccgt atcgtagtta | 7380 |
| tctacacgac ggggagtcag gcaactatgg atgaacgaaa tagacagatc gctgagatag | 7440 |
| gtgcctcact gattaagcat tggtaactgt cagaccaagt ttactcatat atactttaga | 7500 |
| ttgatttaaa acttcatttt taatttaaaa ggatctaggt gaagatcctt tttgataatc | 7560 |
| tcatgaccaa aatcccttaa cgtgagtttt cgttccactg agcgtcagac cccgtagaaa | 7620 |
| agatcaaagg atcttcttga gatccttttt ttctgcgcgt aatctgctgc ttgcaaacaa | 7680 |
| aaaaaccacc gctaccagcg gtggtttgtt tgccggatca agagctacca actctttttc | 7740 |
| cgaaggtaac tggcttcagc agagcgcaga taccaaatac tgtccttcta gtgtagccgt | 7800 |
| agttaggcca ccacttcaag aactctgtag caccgcgtac atacctcgct ctgctaatcc | 7860 |
| tgttaccagt ggctgctgcc agtggcgata agtcgtgtct taccgggttg gactcaagac | 7920 |
| gatagttacc ggataaggcg cagcggtcgg gctgaacggg gggttcgtgc acacagccca | 7980 |
| gcttggagcg aacgacctac accgaactga gatacctaca gcgtgagcta tgagaaagcg | 8040 |
| ccacgcttcc cgaagggaga aaggcggaca ggtatccggt aagcggcagg gtcggaacag | 8100 |
| gagagcgcac gagggagctt ccagggggaa acgcctggta tctttatagt cctgtcgggt | 8160 |
| ttcgccacct ctgacttgag cgtcgatttt tgtgatgctc gtcagggggg cggagcctat | 8220 |
| ggaaaaacgc cagcaacgcg gcctttttac ggttcctggc cttttgctgg ccttttgctc | 8280 |
| acatgttctt tcctgcgtta tcccctgatt ctgtggataa ccgtattacc gggtttgagt | 8340 |
| gagctgatac cgctcgccgc agccgaacga ccgagcgcag cgagtcagtg agcgaccaag | 8400 |
| cggaagagc | 8409 |
| <210> SEQ ID NO: 6 | |
| <211> 8611 | |
| <212> DNA | |
| <213> Adeno-associated virus | |
| <400> 6 | |
| gcccaatacg caaaccgcct ctccccgcgc gttggccgat tcattaatgc agctggcgcg | 60 |
| ctcgctcgct cactgaggcc gcccgggcaa agcccgggcg tcgggcgacc tttggtcgcc | 120 |
| cggcctcagt gagcgagcga gcgcgcagag agggagtggc caactccatc actaggggtt | 180 |
| ccttgtagtt aatgattaac ccgccatgct aattatctac gtagccatgt ctagagttta | 240 |
| aacaagcttg catgtctaag ctagaccctt cagattaaaa ataactgagg taagggcctg | 300 |
| ggtaggggag gtggtgtgag acgctcctgt ctctcctcta tctgcccatc ggccctttgg | 360 |
| ggaggaggaa tgtgcccaag gactaaaaaa aggccatgga gccagagggg cgagggcaac | 420 |
| agacctttca tgggcaaacc ttggggccct gctgtctagc atgccccact acgggtctag | 480 |
| gctgcccatg taaggaggca aggcctgggg acacccgaga tgcctggtta taattaaccc | 540 |
| agacatgtgg ctgccccccc ccccccaaca cctgctgcct ctaaaaataa ccctgtccct | 600 |
| ggtggatccc ctgcatgcga agatcttcga acaaggctgt gggggactga gggcaggctg | 660 |
| taacaggctt gggggccagg gcttatacgt gcctgggact cccaaagtat tactgttcca | 720 |
| tgttcccggc gaagggccag ctgtcccccg ccagctagac tcagcactta gtttaggaac | 780 |
| cagtgagcaa gtcagccctt ggggcagccc atacaaggcc atggggctgg gcaagctgca | 840 |
| cgcctgggtc cggggtgggc acggtgcccg ggcaacgagc tgaaagctca tctgctctca | 900 |
| ggggcccctc cctggggaca gcccctcctg gctagtcaca ccctgtaggc tcctctatat | 960 |
| aacccagggg cacaggggct gccctcattc taccaccacc tccacagcac agacagacac | 1020 |
| tcaggagcca gccagcggcg cgcccaggta agtttagtct ttttgtcttt tatttcaggt | 1080 |
| cccggatccg gtggtggtgc aaatcaaaga actgctcctc agtggatgtt gcctttactt | 1140 |
| ctaggcctgt acggaagtgt tacttctgct ctaaaagctg cggaattgta cccgcggccg | 1200 |
| ccaccatgct gtggtgggag gaggtggagg attgttatga aagggaggac gtgcagaaga | 1260 |
| agacttttac caagtgggtg aacgctcagt tcagcaaatt tgggaagcag cacatcgaga | 1320 |
| atctgttttc cgacctgcag gatgggagac ggctgctgga tctgctggaa ggactgactg | 1380 |
| gccagaagct gcccaaagag aaggggagca ctagggtgca cgccctgaac aacgtgaaca | 1440 |
| aagctctgag agtgctgcag aacaacaacg tggatctggt gaatattggc agtactgata | 1500 |
| tcgtggacgg gaaccacaaa ctgacactgg gcctgatctg gaacattatt ctgcactggc | 1560 |
| aggtgaaaaa tgtgatgaag aacatcatgg ccgggctgca gcagaccaat tccgagaaga | 1620 |
| tcctgctgtc ttgggtgcgg cagagcaccc gcaactatcc ccaggtgaac gtgattaact | 1680 |
| tcactacatc ctggagcgac gggctggccc tgaatgctct gattcacagc cacaggcctg | 1740 |
| atctgttcga ctggaatagc gtggtgtgcc agcagtctgc cacacagcgc ctggaacatg | 1800 |
| ccttcaatat cgctcggtac cagctgggga tcgaaaaact gctggaccca gaggatgtgg | 1860 |
| acactacata cccagataaa aagtctattc tgatgtacat tactagcctg ttccaggtgc | 1920 |
| tgccacagca ggtgtctatt gaagccattc aggaggtgga aatgctgccc cgccccccca | 1980 |
| aagtgactaa agaggagcat tttcagctgc atcatcagat gcattacagc cagcagatta | 2040 |
| ccgtgagcct ggctcaggga tatgagcgca ccagtagtcc aaaaccacgg ttcaagtcct | 2100 |
| acgcttatac ccaggctgcc tacgtgacaa ctagcgaccc tactagatcc ccctttccat | 2160 |
| cccagcacct ggaggcccca gaggacaaga gctttgggtc cagcctgatg gaaagcgagg | 2220 |
| tgaatctgga tcggtaccag acagccctgg aggaggtgct gagctggctg ctgagtgctg | 2280 |
| aagacacact gcaggcccag ggcgaaattt ccaatgacgt ggaagtggtg aaggatcagt | 2340 |
| tccacacaca cgagggctat atgatggacc tgacagctca ccaggggcgc gtgggcaata | 2400 |
| tcctgcagct gggctctaaa ctgatcggca ccgggaaact gagtgaggac gaggaaacag | 2460 |
| aagtgcagga gcagatgaac ctgctgaaca gccgctggga gtgtctgaga gtggctagta | 2520 |
| tggagaagca gtccaacctg caccgggtgc tgatggacct gcagaaccag aaactgaaag | 2580 |
| agctgaacga ctggctgaca aagactgagg aacgcacaag gaagatggag gaggagccac | 2640 |
| tgggacccga cctggaggat ctgaagagac aggtgcagca gcataaggtg ctgcaggagg | 2700 |
| atctggaaca ggagcaggtg cgggtgaact ccctgacaca tatggtggtg gtggtggacg | 2760 |
| aatctagtgg agatcacgcc accgccgccc tggaggaaca gctgaaggtg ctgggggacc | 2820 |
| ggtgggccaa catttgccgg tggaccgagg acaggtgggt gctgctgcag gacatcctgc | 2880 |
| tgaaatggca gaggctgacc gaggagcagt gtctgtttag tgcttggctg agcgagaaag | 2940 |
| aggacgccgt gaacaagatc cacacaaccg gctttaagga tcagaacgaa atgctgtcta | 3000 |
| gcctgcagaa actggctgtg ctgaaggccg atctggagaa aaagaagcag agcatgggca | 3060 |
| aactgtatag cctgaaacag gacctgctga gcaccctgaa gaacaagagc gtgacccaga | 3120 |
| agacagaagc ctggctggat aactttgccc gctgctggga caacctggtg cagaaactgg | 3180 |
| agaaaagtac agctcagatc tctcaggctg tgaccacaac ccagcctagc ctgacccaga | 3240 |
| caaccgtgat ggaaaccgtg accaccgtga caacccgcga acagatcctg gtgaaacatg | 3300 |
| cccaggaaga gctgccacct ccacctcccc agaagaagag aaccctggag cggctgcagg | 3360 |
| agctgcagga agccactgac gaactggacc tgaagctgag gcaggccgaa gtgattaagg | 3420 |
| ggtcttggca gcctgtgggc gatctgctga ttgattccct gcaggaccac ctggaaaagg | 3480 |
| tgaaggctct gagaggcgaa attgctccac tgaaggagaa cgtgagtcat gtgaacgatc | 3540 |
| tggctagaca gctgacaaca ctgggcatcc agctgagccc atacaatctg agcacactgg | 3600 |
| aggacctgaa taccaggtgg aagctgctgc aggtggctgt ggaagaccgg gtgcggcagc | 3660 |
| tgcatgaggc ccatcgcgac ttcggaccag ccagccagca ctttctgagc acatccgtgc | 3720 |
| aggggccctg ggagagggcc atttctccca acaaggtgcc ctactatatt aatcacgaga | 3780 |
| cccagaccac ttgttgggac catcccaaga tgacagaact gtaccagtcc ctggccgatc | 3840 |
| tgaacaacgt gaggtttagc gcttacagaa ccgctatgaa gctgagacgg ctgcagaagg | 3900 |
| ccctgtgcct ggatctgctg tccctgtccg ccgcctgcga tgccctggat cagcataatc | 3960 |
| tgaagcagaa cgatcagcca atggatatcc tgcagatcat caactgcctg accactatct | 4020 |
| acgacaggct ggagcaggag cacaacaacc tggtgaacgt gcctctgtgc gtggatatgt | 4080 |
| gcctgaactg gctgctgaac gtgtatgaca ctgggcgcac cggccggatc agagtgctga | 4140 |
| gttttaaaac tgggattatc tccctgtgta aggcccacct ggaggacaag tacaggtacc | 4200 |
| tgttcaagca ggtggctagt agcactggat tttgtgacca gcgccgcctg ggactgctgc | 4260 |
| tgcatgatag tatccagatt cctagacagc tgggagaggt ggctagtttc ggaggatcta | 4320 |
| acatcgaacc cagcgtgcgc agctgtttcc agtttgccaa taacaaacct gaaatcgagg | 4380 |
| ctgctctgtt cctggattgg atgcgcctgg aaccacagag catggtgtgg ctgcctgtgc | 4440 |
| tgcacagagt ggctgccgcc gaaactgcca agcaccaggc taaatgcaac atctgcaagg | 4500 |
| aatgtcccat tatcggcttt cgctacagga gtctgaaaca ttttaactac gatatttgcc | 4560 |
| agagctgctt cttttccgga agagtggcca aaggacacaa gatgcactac cctatggtgg | 4620 |
| aatattgcac cccaactaca tctggcgaag atgtgcgcga ttttgccaag gtgctgaaga | 4680 |
| ataagtttcg gactaagagg tacttcgcca agcacccccg catggggtat ctgccagtgc | 4740 |
| agacagtgct ggaaggagac aatatggaga ccgatacaat gtgagcggcc gcaataaaag | 4800 |
| atctttattt tcattagatc tgtgtgttgg ttttttgtgt gtctagagca tggctacgta | 4860 |
| gataagtagc atggcgggtt aatcattaac tacaaggaac ccctagtgat ggagttggcc | 4920 |
| actccctctc tgcgcgctcg ctcgctcact gaggccgggc gaccaaaggt cgcccgacgc | 4980 |
| ccgggctttg cccgggcggc ctcagtgagc gagcgagcgc gccagctggc gtaatagcga | 5040 |
| agaggcccgc accgatcgcc cttcccaaca gttgcgcagc ctgaatggcg aatggaagtt | 5100 |
| ccagacgatt gagcgtcaaa atgtaggtat ttccatgagc gtttttcctg ttgcaatggc | 5160 |
| tggcggtaat attgttctgg atattaccag caaggccgat agtttgagtt cttctactca | 5220 |
| ggcaagtgat gttattacta atcaaagaag tattgcgaca acggttaatt tgcgtgatgg | 5280 |
| acagactctt ttactcggtg gcctcactga ttataaaaac acttctcagg attctggcgt | 5340 |
| accgttcctg tctaaaatcc ctttaatcgg cctcctgttt agctcccgct ctgattctaa | 5400 |
| cgaggaaagc acgttatacg tgctcgtcaa agcaaccata gtacgcgccc tgtagcggcg | 5460 |
| cattaagcgc ggcgggtgtg gtggttacgc gcagcgtgac cgctacactt gccagcgccc | 5520 |
| tagcgcccgc tcctttcgct ttcttccctt cctttctcgc cacgttcgcc ggctttcccc | 5580 |
| gtcaagctct aaatcggggg ctccctttag ggttccgatt tagtgattta cggcacctcg | 5640 |
| accccaaaaa acttgattag ggtgatggtt cacgtagtgg gccatcgccc tgatagacgg | 5700 |
| tttttcgccc tttgacgttg gagtccacgt tctttaatag tggactcttg ttccaaactg | 5760 |
| gaacaacact caaccctatc tcggtctatt cttttgattt ataagggatt ttgccgattt | 5820 |
| cggcctattg gttaaaaaat gagctgattt aacaaaaatt taacgcgaat tttaacaaaa | 5880 |
| tattaacgtt tacaatttaa atatttgctt atacaatctt cctgtttttg gggcttttct | 5940 |
| gattatcaac cggggtacat atgattgaca tgctagtttt acgattaccg ttcatcgatt | 6000 |
| ctcttgtttg ctccagactc tcaggcaatg acctgatagc ctttgtagag acctctcaaa | 6060 |
| aatagctacc ctctccggca tgaatttatc agctagaacg gttgaatatc atattgatgg | 6120 |
| tgatttgact gtctccggcc tttctcaccc gtttgaatct ttacctacac attactcagg | 6180 |
| cattgcattt aaaatatatg agggttctaa aaatttttat ccttgcgttg aaataaaggc | 6240 |
| ttctcccgca aaagtattac agggtcataa tgtttttggt acaaccgatt tagctttatg | 6300 |
| ctctgaggct ttattgctta attttgctaa ttctttgcct tgcctgtatg atttattgga | 6360 |
| tgttggaagt tcctgatgcg gtattttctc cttacgcatc tgtgcggtat ttcacaccgc | 6420 |
| atatggtgca ctctcagtac aatctgctct gatgccgcat agttaagcca gccccgacac | 6480 |
| ccgccaacac ccgctgacgc gccctgacgg gcttgtctgc tcccggcatc cgcttacaga | 6540 |
| caagctgtga ccgtctccgg gagctgcatg tgtcagaggt tttcaccgtc atcaccgaaa | 6600 |
| cgcgcgagac gaaagggcct cgtgatacgc ctatttttat aggttaatgt catgataata | 6660 |
| atggtttctt agacgtcagg tggcactttt cggggaaatg tgcgcggaac ccctatttgt | 6720 |
| ttatttttct aaatacattc aaatatgtat ccgctcatga gacaataacc ctgataaatg | 6780 |
| cttcaataat attgaaaaag gaagagtatg agtattcaac atttccgtgt cgcccttatt | 6840 |
| cccttttttg cggcattttg ccttcctgtt tttgctcacc cagaaacgct ggtgaaagta | 6900 |
| aaagatgctg aagatcagtt gggtgcacga gtgggttaca tcgaactgga tctcaacagc | 6960 |
| ggtaagatcc ttgagagttt tcgccccgaa gaacgttttc caatgatgag cacttttaaa | 7020 |
| gttctgctat gtggcgcggt attatcccgt attgacgccg ggcaagagca actcggtcgc | 7080 |
| cgcatacact attctcagaa tgacttggtt gagtactcac cagtcacaga aaagcatctt | 7140 |
| acggatggca tgacagtaag agaattatgc agtgctgcca taaccatgag tgataacact | 7200 |
| gcggccaact tacttctgac aacgatcgga ggaccgaagg agctaaccgc ttttttgcac | 7260 |
| aacatggggg atcatgtaac tcgccttgat cgttgggaac cggagctgaa tgaagccata | 7320 |
| ccaaacgacg agcgtgacac cacgatgcct gtagcaatgg caacaacgtt gcgcaaacta | 7380 |
| ttaactggcg aactacttac tctagcttcc cggcaacaat taatagactg gatggaggcg | 7440 |
| gataaagttg caggaccact tctgcgctcg gcccttccgg ctggctggtt tattgctgat | 7500 |
| aaatctggag ccggtgagcg tgggtctcgc ggtatcattg cagcactggg gccagatggt | 7560 |
| aagccctccc gtatcgtagt tatctacacg acggggagtc aggcaactat ggatgaacga | 7620 |
| aatagacaga tcgctgagat aggtgcctca ctgattaagc attggtaact gtcagaccaa | 7680 |
| gtttactcat atatacttta gattgattta aaacttcatt tttaatttaa aaggatctag | 7740 |
| gtgaagatcc tttttgataa tctcatgacc aaaatccctt aacgtgagtt ttcgttccac | 7800 |
| tgagcgtcag accccgtaga aaagatcaaa ggatcttctt gagatccttt ttttctgcgc | 7860 |
| gtaatctgct gcttgcaaac aaaaaaacca ccgctaccag cggtggtttg tttgccggat | 7920 |
| caagagctac caactctttt tccgaaggta actggcttca gcagagcgca gataccaaat | 7980 |
| actgtccttc tagtgtagcc gtagttaggc caccacttca agaactctgt agcaccgcgt | 8040 |
| acatacctcg ctctgctaat cctgttacca gtggctgctg ccagtggcga taagtcgtgt | 8100 |
| cttaccgggt tggactcaag acgatagtta ccggataagg cgcagcggtc gggctgaacg | 8160 |
| gggggttcgt gcacacagcc cagcttggag cgaacgacct acaccgaact gagataccta | 8220 |
| cagcgtgagc tatgagaaag cgccacgctt cccgaaggga gaaaggcgga caggtatccg | 8280 |
| gtaagcggca gggtcggaac aggagagcgc acgagggagc ttccaggggg aaacgcctgg | 8340 |
| tatctttata gtcctgtcgg gtttcgccac ctctgacttg agcgtcgatt tttgtgatgc | 8400 |
| tcgtcagggg ggcggagcct atggaaaaac gccagcaacg cggccttttt acggttcctg | 8460 |
| gccttttgct ggccttttgc tcacatgttc tttcctgcgt tatcccctga ttctgtggat | 8520 |
| aaccgtatta ccgggtttga gtgagctgat accgctcgcc gcagccgaac gaccgagcgc | 8580 |
| agcgagtcag tgagcgacca agcggaagag c | 8611 |
| <210> SEQ ID NO: 7 | |
| <211> 792 | |
| <212> DNA | |
| <213> Adeno-associated virus | |
| <400> 7 | |
| aagcttgcat gtctaagcta gacccttcag attaaaaata actgaggtaa gggcctgggt | 60 |
| aggggaggtg gtgtgagacg ctcctgtctc tcctctatct gcccatcggc cctttgggga | 120 |
| ggaggaatgt gcccaaggac taaaaaaagg ccatggagcc agaggggcga gggcaacaga | 180 |
| cctttcatgg gcaaaccttg gggccctgct gtctagcatg ccccactacg ggtctaggct | 240 |
| gcccatgtaa ggaggcaagg cctggggaca cccgagatgc ctggttataa ttaacccaga | 300 |
| catgtggctg cccccccccc cccaacacct gctgcctcta aaaataaccc tgtccctggt | 360 |
| ggatcccctg catgcgaaga tcttcgaaca aggctgtggg ggactgaggg caggctgtaa | 420 |
| caggcttggg ggccagggct tatacgtgcc tgggactccc aaagtattac tgttccatgt | 480 |
| tcccggcgaa gggccagctg tcccccgcca gctagactca gcacttagtt taggaaccag | 540 |
| tgagcaagtc agcccttggg gcagcccata caaggccatg gggctgggca agctgcacgc | 600 |
| ctgggtccgg ggtgggcacg gtgcccgggc aacgagctga aagctcatct gctctcaggg | 660 |
| gcccctccct ggggacagcc cctcctggct agtcacaccc tgtaggctcc tctatataac | 720 |
| ccaggggcac aggggctgcc ctcattctac caccacctcc acagcacaga cagacactca | 780 |
| ggagcagcca gc | 792 |
| <210> SEQ ID NO: 8 | |
| <211> 8629 | |
| <212> DNA | |
| <213> Kanamycin Plasmid | |
| <400> 8 | |
| gcgcgctcgc tcgctcactg aggccgcccg ggcaaagccc gggcgtcggg cgacctttgg | 60 |
| tcgcccggcc tcagtgagcg agcgagcgcg cagagaggga gtggccaact ccatcactag | 120 |
| gggttccttg tagttaatga ttaacccgcc atgctactta tctacgtagc catgctctag | 180 |
| agtttaaaca agcttgcatg tctaagctag acccttcaga ttaaaaataa ctgaggtaag | 240 |
| ggcctgggta ggggaggtgg tgtgagacgc tcctgtctct cctctatctg cccatcggcc | 300 |
| ctttggggag gaggaatgtg cccaaggact aaaaaaaggc catggagcca gaggggcgag | 360 |
| ggcaacagac ctttcatggg caaaccttgg ggccctgctg tctagcatgc cccactacgg | 420 |
| gtctaggctg cccatgtaag gaggcaaggc ctggggacac ccgagatgcc tggttataat | 480 |
| taacccagac atgtggctgc cccccccccc ccaacacctg ctgcctctaa aaataaccct | 540 |
| gtccctggtg gatcccctgc atgcgaagat cttcgaacaa ggctgtgggg gactgagggc | 600 |
| aggctgtaac aggcttgggg gccagggctt atacgtgcct gggactccca aagtattact | 660 |
| gttccatgtt cccggcgaag ggccagctgt cccccgccag ctagactcag cacttagttt | 720 |
| aggaaccagt gagcaagtca gcccttgggg cagcccatac aaggccatgg ggctgggcaa | 780 |
| gctgcacgcc tgggtccggg gtgggcacgg tgcccgggca acgagctgaa agctcatctg | 840 |
| ctctcagggg cccctccctg gggacagccc ctcctggcta gtcacaccct gtaggctcct | 900 |
| ctatataacc caggggcaca ggggctgccc tcattctacc accacctcca cagcacagac | 960 |
| agacactcag gagcagccag cggcgcgccc aggtaagttt agtctttttg tcttttattt | 1020 |
| caggtcccgg atccggtggt ggtgcaaatc aaagaactgc tcctcagtgg atgttgcctt | 1080 |
| tacttctagg cctgtacgga agtgttactt ctgctctaaa agctgcggaa ttgtacccgc | 1140 |
| ggccgccacc atgctgtggt gggaggaggt ggaggattgt tatgaaaggg aggacgtgca | 1200 |
| gaagaagact tttaccaagt gggtgaacgc tcagttcagc aaatttggga agcagcacat | 1260 |
| cgagaatctg ttttccgacc tgcaggatgg gagacggctg ctggatctgc tggaaggact | 1320 |
| gactggccag aagctgccca aagagaaggg gagcactagg gtgcacgccc tgaacaacgt | 1380 |
| gaacaaagct ctgagagtgc tgcagaacaa caacgtggat ctggtgaata ttggcagtac | 1440 |
| tgatatcgtg gacgggaacc acaaactgac actgggcctg atctggaaca ttattctgca | 1500 |
| ctggcaggtg aaaaatgtga tgaagaacat catggccggg ctgcagcaga ccaattccga | 1560 |
| gaagatcctg ctgtcttggg tgcggcagag cacccgcaac tatccccagg tgaacgtgat | 1620 |
| taacttcact acatcctgga gcgacgggct ggccctgaat gctctgattc acagccacag | 1680 |
| gcctgatctg ttcgactgga atagcgtggt gtgccagcag tctgccacac agcgcctgga | 1740 |
| acatgccttc aatatcgctc ggtaccagct ggggatcgaa aaactgctgg acccagagga | 1800 |
| tgtggacact acatacccag ataaaaagtc tattctgatg tacattacta gcctgttcca | 1860 |
| ggtgctgcca cagcaggtgt ctattgaagc cattcaggag gtggaaatgc tgccccgccc | 1920 |
| ccccaaagtg actaaagagg agcattttca gctgcatcat cagatgcatt acagccagca | 1980 |
| gattaccgtg agcctggctc agggatatga gcgcaccagt agtccaaaac cacggttcaa | 2040 |
| gtcctacgct tatacccagg ctgcctacgt gacaactagc gaccctacta gatccccctt | 2100 |
| tccatcccag cacctggagg ccccagagga caagagcttt gggtccagcc tgatggaaag | 2160 |
| cgaggtgaat ctggatcggt accagacagc cctggaggag gtgctgagct ggctgctgag | 2220 |
| tgctgaagac acactgcagg cccagggcga aatttccaat gacgtggaag tggtgaagga | 2280 |
| tcagttccac acacacgagg gctatatgat ggacctgaca gctcaccagg ggcgcgtggg | 2340 |
| caatatcctg cagctgggct ctaaactgat cggcaccggg aaactgagtg aggacgagga | 2400 |
| aacagaagtg caggagcaga tgaacctgct gaacagccgc tgggagtgtc tgagagtggc | 2460 |
| tagtatggag aagcagtcca acctgcaccg ggtgctgatg gacctgcaga accagaaact | 2520 |
| gaaagagctg aacgactggc tgacaaagac tgaggaacgc acaaggaaga tggaggagga | 2580 |
| gccactggga cccgacctgg aggatctgaa gagacaggtg cagcagcata aggtgctgca | 2640 |
| ggaggatctg gaacaggagc aggtgcgggt gaactccctg acacatatgg tggtggtggt | 2700 |
| ggacgaatct agtggagatc acgccaccgc cgccctggag gaacagctga aggtgctggg | 2760 |
| ggaccggtgg gccaacattt gccggtggac cgaggacagg tgggtgctgc tgcaggacat | 2820 |
| cctgctgaaa tggcagaggc tgaccgagga gcagtgtctg tttagtgctt ggctgagcga | 2880 |
| gaaagaggac gccgtgaaca agatccacac aaccggcttt aaggatcaga acgaaatgct | 2940 |
| gtctagcctg cagaaactgg ctgtgctgaa ggccgatctg gagaaaaaga agcagagcat | 3000 |
| gggcaaactg tatagcctga aacaggacct gctgagcacc ctgaagaaca agagcgtgac | 3060 |
| ccagaagaca gaagcctggc tggataactt tgcccgctgc tgggacaacc tggtgcagaa | 3120 |
| actggagaaa agtacagctc agatctctca ggctgtgacc acaacccagc ctagcctgac | 3180 |
| ccagacaacc gtgatggaaa ccgtgaccac cgtgacaacc cgcgaacaga tcctggtgaa | 3240 |
| acatgcccag gaagagctgc cacctccacc tccccagaag aagagaaccc tggagcggct | 3300 |
| gcaggagctg caggaagcca ctgacgaact ggacctgaag ctgaggcagg ccgaagtgat | 3360 |
| taaggggtct tggcagcctg tgggcgatct gctgattgat tccctgcagg accacctgga | 3420 |
| aaaggtgaag gctctgagag gcgaaattgc tccactgaag gagaacgtga gtcatgtgaa | 3480 |
| cgatctggct agacagctga caacactggg catccagctg agcccataca atctgagcac | 3540 |
| actggaggac ctgaatacca ggtggaagct gctgcaggtg gctgtggaag accgggtgcg | 3600 |
| gcagctgcat gaggcccatc gcgacttcgg accagccagc cagcactttc tgagcacatc | 3660 |
| cgtgcagggg ccctgggaga gggccatttc tcccaacaag gtgccctact atattaatca | 3720 |
| cgagacccag accacttgtt gggaccatcc caagatgaca gaactgtacc agtccctggc | 3780 |
| cgatctgaac aacgtgaggt ttagcgctta cagaaccgct atgaagctga gacggctgca | 3840 |
| gaaggccctg tgcctggatc tgctgtccct gtccgccgcc tgcgatgccc tggatcagca | 3900 |
| taatctgaag cagaacgatc agccaatgga tatcctgcag atcatcaact gcctgaccac | 3960 |
| tatctacgac aggctggagc aggagcacaa caacctggtg aacgtgcctc tgtgcgtgga | 4020 |
| tatgtgcctg aactggctgc tgaacgtgta tgacactggg cgcaccggcc ggatcagagt | 4080 |
| gctgagtttt aaaactggga ttatctccct gtgtaaggcc cacctggagg acaagtacag | 4140 |
| gtacctgttc aagcaggtgg ctagtagcac tggattttgt gaccagcgcc gcctgggact | 4200 |
| gctgctgcat gatagtatcc agattcctag acagctggga gaggtggcta gtttcggagg | 4260 |
| atctaacatc gaacccagcg tgcgcagctg tttccagttt gccaataaca aacctgaaat | 4320 |
| cgaggctgct ctgttcctgg attggatgcg cctggaacca cagagcatgg tgtggctgcc | 4380 |
| tgtgctgcac agagtggctg ccgccgaaac tgccaagcac caggctaaat gcaacatctg | 4440 |
| caaggaatgt cccattatcg gctttcgcta caggagtctg aaacatttta actacgatat | 4500 |
| ttgccagagc tgcttctttt ccggaagagt ggccaaagga cacaagatgc actaccctat | 4560 |
| ggtggaatat tgcaccccaa ctacatctgg cgaagatgtg cgcgattttg ccaaggtgct | 4620 |
| gaagaataag tttcggacta agaggtactt cgccaagcac ccccgcatgg ggtatctgcc | 4680 |
| agtgcagaca gtgctggaag gagacaatat ggagaccgat acaatgtgag cggccgcaat | 4740 |
| aaaagatctt tattttcatt agatctgtgt gttggttttt tgtgtgtcta gagtcgacca | 4800 |
| gagcatggct acgtagataa gtagcatggc gggttaatca ttaactacaa ggaaccccta | 4860 |
| gtgatggagt tggccactcc ctctctgcgc gctcgctcgc tcactgaggc cgggcgacca | 4920 |
| aaggtcgccc gacgcccggg ctttgcccgg gcggcctcag tgagcgagcg agcgcgcagc | 4980 |
| tgcattaatg aatcggccaa cgcgcgggga gaggcggttt gcgtattggg cgctcttccg | 5040 |
| cttcctcgct cactgactcg ctgcgctcgg tcgttcggct gcggcgagcg gtatcagctc | 5100 |
| actcaaaggc ggtaatacgg ttatccacag aatcagggga taacgcagga aagaacatgt | 5160 |
| gagcaaaagg ccagcaaaag gccaggaacc gtaaaaaggc cgcgttgctg gcgtttttcc | 5220 |
| ataggctccg cccccctgac gagcatcaca aaaatcgacg ctcaagtcag aggtggcgaa | 5280 |
| acccgacagg actataaaga taccaggcgt ttccccctgg aagctccctc gtgcgctctc | 5340 |
| ctgttccgac cctgccgctt accggatacc tgtccgcctt tctcccttcg ggaagcgtgg | 5400 |
| cgctttctca tagctcacgc tgtaggtatc tcagttcggt gtaggtcgtt cgctccaagc | 5460 |
| tgggctgtgt gcacgaaccc cccgttcagc ccgaccgctg cgccttatcc ggtaactatc | 5520 |
| gtcttgagtc caacccggta agacacgact tatcgccact ggcagcagcc actggtaaca | 5580 |
| ggattagcag agcgaggtat gtaggcggtg ctacagagtt cttgaagtgg tggcctaact | 5640 |
| acggctacac tagaagaaca gtatttggta tctgcgctct gctgaagcca gttaccttcg | 5700 |
| gaaaaagagt tggtagctct tgatccggca aacaaaccac cgctggtagc ggtggttttt | 5760 |
| ttgtttgcaa gcagcagatt acgcgcagaa aaaaaggatc tcaagaagat cctttgatct | 5820 |
| tttctacggg gtctgacgct cagtggaacg aaaactcacg ttaagggatt ttggtcatga | 5880 |
| gattatcaaa aaggatcttc acctagatcc ttttaaatta aaaatgaagt tttaaatcaa | 5940 |
| tctaaagtat atatgagtaa aaatattccg gaattgccag ctggggcgcc ctctggtaag | 6000 |
| gttgggaagc cctgcaaagt aaactggatg gctttcttgc cgccaaggat ctgatggcgc | 6060 |
| aggggatcaa gatctgatca agagacagga tgaggatcgt ttcgcatgat tgaacaagat | 6120 |
| ggattgcacg caggttctcc ggccgcttgg gtggagaggc tattcggcta tgactgggca | 6180 |
| caacagacaa tcggctgctc tgatgccgcc gtgttccggc tgtcagcgca ggggcgcccg | 6240 |
| gttctttttg tcaagaccga cctgtccggt gccctgaatg aactgcagga cgaggcagcg | 6300 |
| cggctatcgt ggctggccac gacgggcgtt ccttgcgcag ctgtgctcga cgttgtcact | 6360 |
| gaagcgggaa gggactggct gctattgggc gaagtgccgg ggcaggatct cctgtcatcc | 6420 |
| caccttgctc ctgccgagaa agtatccatc atggctgatg caatgcggcg gctgcatacg | 6480 |
| cttgatccgg ctacctgccc attcgaccac caagcgaaac atcgcatcga gcgagcacgt | 6540 |
| actcggatgg aagccggtct tgtcgatcag gatgatctgg acgaagagca tcaggggctc | 6600 |
| gcgccagccg aactgttcgc caggctcaag gcgcgcatgc ccgacggcga ggatctcgtc | 6660 |
| gtgacccatg gcgatgcctg cttgccgaat atcatggtgg aaaatggccg cttttctgga | 6720 |
| ttcatcgact gtggccggct gggtgtggcg gaccgctatc aggacatagc gttggctacc | 6780 |
| cgtgatattg ctgaagagct tggcggcgaa tgggctgacc gcttcctcgt gctttacggt | 6840 |
| atcgccgctc ccgattcgca gcgcatcgcc ttctatcgcc ttcttgacga gttcttctga | 6900 |
| accggtaata ttattgaagc atttatcagg gttattgtct catgagcgga tacatatttg | 6960 |
| aatgtattta gaaaaataaa caaatagggg ttccgcgcac atttccccga aaagtgccac | 7020 |
| ctgacgtcta agaaaccatt attatcatga cattaaccta taaaaatagg cgtatcacga | 7080 |
| ggccctttcg tctcgcgcgt ttcggtgatg acggtgaaaa cctctgacac atgcagctcc | 7140 |
| cggagacggt cacagcttgt ctgtaagcgg atgccgggag cagacaagcc cgtcagggcg | 7200 |
| cgtcagcggg tgttggcggg tgtcggggct ggcttaacta tgcggcatca gagcagattg | 7260 |
| tactgagagt gcaccatatg cggtgtgaaa taccgcacag atgcgtaagg agaaaatacc | 7320 |
| gcatcaggcg attccaacat ccaataaatc atacaggcaa ggcaaagaat tagcaaaatt | 7380 |
| aagcaataaa gcctcagagc ataaagctaa atcggttgta ccaaaaacat tatgaccctg | 7440 |
| taatactttt gcgggagaag cctttatttc aacgcaagga taaaaatttt tagaaccctc | 7500 |
| atatatttta aatgcaatgc ctgagtaatg tgtaggtaaa gattcaaacg ggtgagaaag | 7560 |
| gccggagaca gtcaaatcac catcaatatg atattcaacc gttctagctg ataaattcat | 7620 |
| gccggagagg gtagctattt ttgagaggtc tctacaaagg ctatcaggtc attgcctgag | 7680 |
| agtctggagc aaacaagaga atcgatgaac ggtaatcgta aaactagcat gtcaatcata | 7740 |
| tgtaccccgg ttgataatca gaaaagcccc aaaaacagga agattgtata agcaaatatt | 7800 |
| taaattgtaa gcgttaatat tttgttaaaa ttcgcgttaa atttttgtta aatcagctca | 7860 |
| ttttttaacc aataggccga aatcggcaaa atcccttata aatcaaaaga atagaccgag | 7920 |
| atagggttga gtgttgttcc agtttggaac aagagtccac tattaaagaa cgtggactcc | 7980 |
| aacgtcaaag ggcgaaaaac cgtctatcag ggcgatggcc cactacgtga accatcaccc | 8040 |
| taatcaagtt ttttggggtc gaggtgccgt aaagcactaa atcggaaccc taaagggagc | 8100 |
| ccccgattta gagcttgacg gggaaagccg gcgaacgtgg cgagaaagga agggaagaaa | 8160 |
| gcgaaaggag cgggcgctag ggcgctggca agtgtagcgg tcacgctgcg cgtaaccacc | 8220 |
| acacccgccg cgcttaatgc gccgctacag ggcgcgtact atggttgctt tgacgagcac | 8280 |
| gtataacgtg ctttcctcgt tagaatcaga gcgggagcta aacaggaggc cgattaaagg | 8340 |
| gattttagac aggaacggta cgccagaatc ctgagaagtg tttttataat cagtgaggcc | 8400 |
| accgagtaaa agagtctgtc catcacgcaa attaaccgtt gtcgcaatac ttctttgatt | 8460 |
| agtaataaca tcacttgcct gagtagaaga actcaaacta tcggccttgc tggtaatatc | 8520 |
| cagaacaata ttaccgccag ccattgcaac ggaatcgcca ttcgccattc aggctgcgca | 8580 |
| actgttggga agggcgatcg gtgcgggcct cttcgctatt acgccagct | 8629 |
| <210> SEQ ID NO: 9 | |
| <211> 4977 | |
| <212> DNA | |
| <213> Kanamycin Cassatte | |
| <400> 9 | |
| gcgcgctcgc tcgctcactg aggccgcccg ggcaaagccc gggcgtcggg cgacctttgg | 60 |
| tcgcccggcc tcagtgagcg agcgagcgcg cagagaggga gtggccaact ccatcactag | 120 |
| gggttccttg tagttaatga ttaacccgcc atgctactta tctacgtagc catgctctag | 180 |
| agtttaaaca agcttgcatg tctaagctag acccttcaga ttaaaaataa ctgaggtaag | 240 |
| ggcctgggta ggggaggtgg tgtgagacgc tcctgtctct cctctatctg cccatcggcc | 300 |
| ctttggggag gaggaatgtg cccaaggact aaaaaaaggc catggagcca gaggggcgag | 360 |
| ggcaacagac ctttcatggg caaaccttgg ggccctgctg tctagcatgc cccactacgg | 420 |
| gtctaggctg cccatgtaag gaggcaaggc ctggggacac ccgagatgcc tggttataat | 480 |
| taacccagac atgtggctgc cccccccccc ccaacacctg ctgcctctaa aaataaccct | 540 |
| gtccctggtg gatcccctgc atgcgaagat cttcgaacaa ggctgtgggg gactgagggc | 600 |
| aggctgtaac aggcttgggg gccagggctt atacgtgcct gggactccca aagtattact | 660 |
| gttccatgtt cccggcgaag ggccagctgt cccccgccag ctagactcag cacttagttt | 720 |
| aggaaccagt gagcaagtca gcccttgggg cagcccatac aaggccatgg ggctgggcaa | 780 |
| gctgcacgcc tgggtccggg gtgggcacgg tgcccgggca acgagctgaa agctcatctg | 840 |
| ctctcagggg cccctccctg gggacagccc ctcctggcta gtcacaccct gtaggctcct | 900 |
| ctatataacc caggggcaca ggggctgccc tcattctacc accacctcca cagcacagac | 960 |
| agacactcag gagcagccag cggcgcgccc aggtaagttt agtctttttg tcttttattt | 1020 |
| caggtcccgg atccggtggt ggtgcaaatc aaagaactgc tcctcagtgg atgttgcctt | 1080 |
| tacttctagg cctgtacgga agtgttactt ctgctctaaa agctgcggaa ttgtacccgc | 1140 |
| ggccgccacc atgctgtggt gggaggaggt ggaggattgt tatgaaaggg aggacgtgca | 1200 |
| gaagaagact tttaccaagt gggtgaacgc tcagttcagc aaatttggga agcagcacat | 1260 |
| cgagaatctg ttttccgacc tgcaggatgg gagacggctg ctggatctgc tggaaggact | 1320 |
| gactggccag aagctgccca aagagaaggg gagcactagg gtgcacgccc tgaacaacgt | 1380 |
| gaacaaagct ctgagagtgc tgcagaacaa caacgtggat ctggtgaata ttggcagtac | 1440 |
| tgatatcgtg gacgggaacc acaaactgac actgggcctg atctggaaca ttattctgca | 1500 |
| ctggcaggtg aaaaatgtga tgaagaacat catggccggg ctgcagcaga ccaattccga | 1560 |
| gaagatcctg ctgtcttggg tgcggcagag cacccgcaac tatccccagg tgaacgtgat | 1620 |
| taacttcact acatcctgga gcgacgggct ggccctgaat gctctgattc acagccacag | 1680 |
| gcctgatctg ttcgactgga atagcgtggt gtgccagcag tctgccacac agcgcctgga | 1740 |
| acatgccttc aatatcgctc ggtaccagct ggggatcgaa aaactgctgg acccagagga | 1800 |
| tgtggacact acatacccag ataaaaagtc tattctgatg tacattacta gcctgttcca | 1860 |
| ggtgctgcca cagcaggtgt ctattgaagc cattcaggag gtggaaatgc tgccccgccc | 1920 |
| ccccaaagtg actaaagagg agcattttca gctgcatcat cagatgcatt acagccagca | 1980 |
| gattaccgtg agcctggctc agggatatga gcgcaccagt agtccaaaac cacggttcaa | 2040 |
| gtcctacgct tatacccagg ctgcctacgt gacaactagc gaccctacta gatccccctt | 2100 |
| tccatcccag cacctggagg ccccagagga caagagcttt gggtccagcc tgatggaaag | 2160 |
| cgaggtgaat ctggatcggt accagacagc cctggaggag gtgctgagct ggctgctgag | 2220 |
| tgctgaagac acactgcagg cccagggcga aatttccaat gacgtggaag tggtgaagga | 2280 |
| tcagttccac acacacgagg gctatatgat ggacctgaca gctcaccagg ggcgcgtggg | 2340 |
| caatatcctg cagctgggct ctaaactgat cggcaccggg aaactgagtg aggacgagga | 2400 |
| aacagaagtg caggagcaga tgaacctgct gaacagccgc tgggagtgtc tgagagtggc | 2460 |
| tagtatggag aagcagtcca acctgcaccg ggtgctgatg gacctgcaga accagaaact | 2520 |
| gaaagagctg aacgactggc tgacaaagac tgaggaacgc acaaggaaga tggaggagga | 2580 |
| gccactggga cccgacctgg aggatctgaa gagacaggtg cagcagcata aggtgctgca | 2640 |
| ggaggatctg gaacaggagc aggtgcgggt gaactccctg acacatatgg tggtggtggt | 2700 |
| ggacgaatct agtggagatc acgccaccgc cgccctggag gaacagctga aggtgctggg | 2760 |
| ggaccggtgg gccaacattt gccggtggac cgaggacagg tgggtgctgc tgcaggacat | 2820 |
| cctgctgaaa tggcagaggc tgaccgagga gcagtgtctg tttagtgctt ggctgagcga | 2880 |
| gaaagaggac gccgtgaaca agatccacac aaccggcttt aaggatcaga acgaaatgct | 2940 |
| gtctagcctg cagaaactgg ctgtgctgaa ggccgatctg gagaaaaaga agcagagcat | 3000 |
| gggcaaactg tatagcctga aacaggacct gctgagcacc ctgaagaaca agagcgtgac | 3060 |
| ccagaagaca gaagcctggc tggataactt tgcccgctgc tgggacaacc tggtgcagaa | 3120 |
| actggagaaa agtacagctc agatctctca ggctgtgacc acaacccagc ctagcctgac | 3180 |
| ccagacaacc gtgatggaaa ccgtgaccac cgtgacaacc cgcgaacaga tcctggtgaa | 3240 |
| acatgcccag gaagagctgc cacctccacc tccccagaag aagagaaccc tggagcggct | 3300 |
| gcaggagctg caggaagcca ctgacgaact ggacctgaag ctgaggcagg ccgaagtgat | 3360 |
| taaggggtct tggcagcctg tgggcgatct gctgattgat tccctgcagg accacctgga | 3420 |
| aaaggtgaag gctctgagag gcgaaattgc tccactgaag gagaacgtga gtcatgtgaa | 3480 |
| cgatctggct agacagctga caacactggg catccagctg agcccataca atctgagcac | 3540 |
| actggaggac ctgaatacca ggtggaagct gctgcaggtg gctgtggaag accgggtgcg | 3600 |
| gcagctgcat gaggcccatc gcgacttcgg accagccagc cagcactttc tgagcacatc | 3660 |
| cgtgcagggg ccctgggaga gggccatttc tcccaacaag gtgccctact atattaatca | 3720 |
| cgagacccag accacttgtt gggaccatcc caagatgaca gaactgtacc agtccctggc | 3780 |
| cgatctgaac aacgtgaggt ttagcgctta cagaaccgct atgaagctga gacggctgca | 3840 |
| gaaggccctg tgcctggatc tgctgtccct gtccgccgcc tgcgatgccc tggatcagca | 3900 |
| taatctgaag cagaacgatc agccaatgga tatcctgcag atcatcaact gcctgaccac | 3960 |
| tatctacgac aggctggagc aggagcacaa caacctggtg aacgtgcctc tgtgcgtgga | 4020 |
| tatgtgcctg aactggctgc tgaacgtgta tgacactggg cgcaccggcc ggatcagagt | 4080 |
| gctgagtttt aaaactggga ttatctccct gtgtaaggcc cacctggagg acaagtacag | 4140 |
| gtacctgttc aagcaggtgg ctagtagcac tggattttgt gaccagcgcc gcctgggact | 4200 |
| gctgctgcat gatagtatcc agattcctag acagctggga gaggtggcta gtttcggagg | 4260 |
| atctaacatc gaacccagcg tgcgcagctg tttccagttt gccaataaca aacctgaaat | 4320 |
| cgaggctgct ctgttcctgg attggatgcg cctggaacca cagagcatgg tgtggctgcc | 4380 |
| tgtgctgcac agagtggctg ccgccgaaac tgccaagcac caggctaaat gcaacatctg | 4440 |
| caaggaatgt cccattatcg gctttcgcta caggagtctg aaacatttta actacgatat | 4500 |
| ttgccagagc tgcttctttt ccggaagagt ggccaaagga cacaagatgc actaccctat | 4560 |
| ggtggaatat tgcaccccaa ctacatctgg cgaagatgtg cgcgattttg ccaaggtgct | 4620 |
| gaagaataag tttcggacta agaggtactt cgccaagcac ccccgcatgg ggtatctgcc | 4680 |
| agtgcagaca gtgctggaag gagacaatat ggagaccgat acaatgtgag cggccgcaat | 4740 |
| aaaagatctt tattttcatt agatctgtgt gttggttttt tgtgtgtcta gagtcgacca | 4800 |
| gagcatggct acgtagataa gtagcatggc gggttaatca ttaactacaa ggaaccccta | 4860 |
| gtgatggagt tggccactcc ctctctgcgc gctcgctcgc tcactgaggc cgggcgacca | 4920 |
| aaggtcgccc gacgcccggg ctttgcccgg gcggcctcag tgagcgagcg agcgcgc | 4977 |
| SEQ ID NO: 44 (corresponding to SEQ ID NO: 19 of PCT/US2020/019892). | |
| ctgcgcgctc gctcgctcac tgaggccgcc cgggcaaagc ccgggcgtcg ggcgaccttt | 60 |
| ggtcgcccgg cctcagtgag cgagcgagcg cgcagagagg gagtggggtt aaccaattgg | 120 |
| cggccgcaag cttgcatgtc taagctagac ccttcagatt aaaaataact gaggtaaggg | 180 |
| cctgggtagg ggaggtggtg tgagacgctc ctgtctctcc tctatctgcc catcggccct | 240 |
| ttggggagga ggaatgtgcc caaggactaa aaaaaggcca tggagccaga ggggcgaggg | 300 |
| caacagacct ttcatgggca aaccttgggg ccctgctgtc tagcatgccc cactacgggt | 360 |
| ctaggctgcc catgtaagga ggcaaggcct ggggacaccc gagatgcctg gttataatta | 420 |
| acccagacat gtggctgccc cccccccccc aacacctgct gcctctaaaa ataaccctgt | 480 |
| ccctggtgga tcccctgcat gcgaagatct tcgaacaagg ctgtggggga ctgagggcag | 540 |
| gctgtaacag gcttgggggc cagggcttat acgtgcctgg gactcccaaa gtattactgt | 600 |
| tccatgttcc cggcgaaggg ccagctgtcc cccgccagct agactcagca cttagtttag | 660 |
| gaaccagtga gcaagtcagc ccttggggca gcccatacaa ggccatgggg ctgggcaagc | 720 |
| tgcacgcctg ggtccggggt gggcacggtg cccgggcaac gagctgaaag ctcatctgct | 780 |
| ctcaggggcc cctccctggg gacagcccct cctggctagt cacaccctgt aggctcctct | 840 |
| atataaccca ggggcacagg ggctgccctc attctaccac cacctccaca gcacagacag | 900 |
| acactcagga gcagccagcg gcgcgcccag gtaagtttag tctttttgtc ttttatttca | 960 |
| ggtcccggat ccggtggtgg tgcaaatcaa agaactgctc ctcagtggat gttgccttta | 1020 |
| cttctaggcc tgtacggaag tgttacttct gctctaaaag ctgcggaatt gtacccggta | 1080 |
| ccaccatggc agcagcagcc gccgcagccg ccgagcagca gtcaagcaat ggaccagtga | 1140 |
| aaaaatcaat gagagaaaaa gccgtcgaga ggagatcagt gaataaggag cacaacagca | 1200 |
| atttcaaagc cggctacatc cctattgacg aagatcgcct gcataagaca ggcctgaggg | 1260 |
| ggcgcaaagg aaacctggca atctgcgtca tcattctgct gtttatcctg gccgtgatta | 1320 |
| atctgatcat tactctggtg atttgggctg tcatccgcat tggcccaaac gggtgtgact | 1380 |
| ctatggagtt ccacgaaagt ggcctgctgc gatttaagca ggtgtccgat atgggggtca | 1440 |
| tccatccact gtacaaatct actgtcggcg ggcggagaaa cgagaatctg gtgatcaccg | 1500 |
| ggaacaatca gcccattgtg ttccagcagg gaaccacaaa gctgtctgtg gaaaacaata | 1560 |
| aaacatcaat cactagcgac attggcatgc agttctttga tccccggacc cagaatatcc | 1620 |
| tgttcagtac cgactatgag acacacgaat ttcatctgcc ttccggggtg aagtctctga | 1680 |
| acgtccagaa agccagcact gagagaatca ccagtaacgc tacatcagac ctgaatatca | 1740 |
| aggtggatgg acgagctatt gtccggggaa atgagggcgt gttcatcatg ggcaagacaa | 1800 |
| ttgaatttca catgggaggc aacatggagc tgaaagcaga aaacagcatc attctgaatg | 1860 |
| ggagcgtgat ggtctccact accagactgc ccagctcctc tagtggagac cagctggggt | 1920 |
| ccggagattg ggtcaggtat aagctgtgca tgtgtgccga tggcaccctg tttaaagtgc | 1980 |
| aggtcaccag ccagaatatg ggatgtcaga ttagcgataa cccttgtggg aatactcatt | 2040 |
| aaaagcttgg ccgcaataaa agatctttat tttcattaga tctgtgtgtt ggttttttgt | 2100 |
| gtgtcctgca ggggcgcgcc tctagagcat ggctacgtag ataagtagca tggcgggtta | 2160 |
| atcattaact acaaggaacc cctagtgatg gagttggcca ctccctctct gcgcgctcgc | 2220 |
| tcgctcactg aggccgggcg accaaaggtc gcccgacgcc cgggctttgc ccgggcggcc | 2280 |
| tcagtgagcg agcgagcgcg cag | |
| SEQ ID NO: 45: (SEQ ID NO: 1 of PCT/US2020/019892) | |
| Beta-sarcoglycan | |
| atggcagcag cagccgccgc agccgccgag cagcagtcaa gcaatggacc agtgaaaaaa | |
| tcaatgagag aaaaagccgt cgagaggaga tcagtgaata aggagcacaa cagcaatttc | |
| aaagccggct acatccctat tgacgaagat cgcctgcata agacaggcct gagggggcgc | |
| aaaggaaacc tggcaatctg cgtcatcatt ctgctgttta tcctggccgt gattaatctg | |
| atcattactc tggtgatttg ggctgtcatc cgcattggcc caaacgggtg tgactctatg | |
| gagttccacg aaagtggcct gctgcgattt aagcaggtgt ccgatatggg ggtcatccat | |
| ccactgtaca aatctactgt cggcgggcgg agaaacgaga atctggtgat caccgggaac | |
| aatcagccca ttgtgttcca gcagggaacc acaaagctgt ctgtggaaaa caataaaaca | |
| tcaatcacta gcgacattgg catgcagttc tttgatcccc ggacccagaa tatcctgttc | |
| agtaccgact atgagacaca cgaatttcat ctgccttccg gggtgaagtc tctgaacgtc | |
| cagaaagcca gcactgagag aatcaccagt aacgctacat cagacctgaa tatcaaggtg | |
| gatggacgag ctattgtccg gggaaatgag ggcgtgttca tcatgggcaa gacaattgaa | |
| tttcacatgg gaggcaacat ggagctgaaa gcagaaaaca gcatcattct gaatgggagc | |
| gtgatggtct ccactaccag actgcccagc tcctctagtg gagaccagct ggggtccgga | |
| gattgggtca ggtataagct gtgcatgtgt gccgatggca ccctgtttaa agtgcaggtc | |
| accagccaga atatgggatg tcagattagc gataaccctt gtgggaatac tcattaa | |
| SEQ ID NO: 46 (SEQ ID NO: 2 of PCT/US2020/019892) | |
| Beta-sarcoglycan | |
| Met Ala Ala Ala Ala Ala Ala Ala Ala Glu Gln Gln Ser Ser Asn Gly | |
| Pro Val Lys Lys Ser Met Arg Glu Lys Ala Val Glu Arg Arg Ser Val | |
| Asn Lys Glu His Asn Ser Asn Phe Lys Ala Gly Tyr Ile Pro Ile Asp | |
| Glu Asp Arg Leu His Lys Thr Gly Leu Arg Gly Arg Lys Gly Asn Leu | |
| Ala Ile Cys Val Ile Ile Leu Leu Phe Ile Leu Ala Val Ile Asn Leu | |
| Ile Ile Thr Leu Val Ile Trp Ala Val Ile Arg Ile Gly Pro Asn Gly | |
| Cys Asp Ser Met Glu Phe His Glu Ser Gly Leu Leu Arg Phe Lys Gln | |
| Val Ser Asp Met Gly Val Ile His Pro Leu Tyr Lys Ser Thr Val Gly | |
| Gly Arg Arg Asn Glu Asn Leu Val Ile Thr Gly Asn Asn Gln Pro Ile | |
| Val Phe Gln Gln Gly Thr Thr Lys Leu Ser Val Glu Asn Asn Lys Thr | |
| Ser Ile Thr Ser Asp Ile Gly Met Gln Phe Phe Asp Pro Arg Thr Gln | |
| Asn Ile Leu Phe Ser Thr Asp Tyr Glu Thr His Glu Phe His Leu Pro | |
| Ser Gly Val Lys Ser Leu Asn Val Gln Lys Ala Ser Thr Glu Arg Ile | |
| Thr Ser Asn Ala Thr Ser Asp Leu Asn Ile Lys Val Asp Gly Arg Ala | |
| Ile Val Arg Gly Asn Glu Gly Val Phe Ile Met Gly Lys Thr Ile Glu | |
| Phe His Met Gly Gly Asn Met Glu Leu Lys Ala Glu Asn Ser Ile Ile | |
| Leu Asn Gly Ser Val Met Val Ser Thr Thr Arg Leu Pro Ser Ser Ser | |
| Ser Gly Asp Gln Leu Gly Ser Gly Asp Trp Val Arg Tyr Lys Leu Cys | |
| Met Cys Ala Asp Gly Thr Leu Phe Lys Val Gln Val Thr Ser Gln Asn | |
| Met Gly Cys Gln Ile Ser Asp Asn Pro Cys Gly Asn Thr His | |

1. A method of treating muscular dystrophy in a human subject in need thereof comprising the step of administering a recombinant adeno-virus associated (rAAV) and an anti-inflammatory steroid, wherein the rAAV is selected from the group consisting of rAAV.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05.

2-20. (canceled)

21. A method of treating a muscular dystrophy in a human subject in need thereof comprising administering a recombinant adeno-virus associated (rAAV); and an immunosuppressing regimen, wherein the immunosuppressing regimen comprises administering one or more of an anti-inflammatory steroid, an anti-CD20 antibody, and an immunosuppressing macrolide and wherein the rAAV is selected from the group consisting of: AAVrh.74.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05.

22-23. (canceled)

24. The method of claim 21 wherein the immunosuppressing regimen comprises administering an anti-inflammatory steroid, an anti-CD20 antibody, and an immunosuppressing macrolide.

25. The method of claim 21 wherein the anti-inflammatory steroid is
(a) administered about 24 hours prior to administration of the rAAV or
(b) administered prior to administration of the rAAV and the anti-inflammatory steroid is administered at least once a day from day 1 to 30 days after administration of the rAAV.

26-27. (canceled)

28. The method of claim 21, wherein the anti-inflammatory steroid is a glucocorticoid.

29. The method of claim 21 wherein the anti-inflammatory steroid is prednisone, prednisolone, betamethasone, dexamethasone, hydrocortisone, methylprednisolone or deflazacort.

30. The method of claim 21 wherein the anti-CD20 specific antibody prior to administration of the rAAV.

31. The method of claim 30 wherein the anti-CD20 specific antibody is
(a) administered at least 7 days prior to administration of the rAAV, or
(b) administered about 14 days prior to administration of the rAAV, administered about 7 days prior to administration of the rAAV and within about 24 hours of the administration of the rAAV.

32. (canceled)

33. The method of claim 21, wherein the immunosuppressing regimen further comprises administering an anti-CD20 specific antibody after administration of the rAAV.

34. (canceled)

35. The method of claim 21 wherein the anti-CD20 specific antibody is rituximab, ocrelizumab or ofatumumab.

36. The method of claim 21 wherein the immunosuppressing macrolide is administered at least once a day for at least three days prior to administration of the rAAV.

37. The method of claim 21 wherein the immunosuppressing regimen further comprises administering an immunosuppressing macrolide after administration of the rAAV.

38. (canceled)

39. The method of claim 21 wherein the immunosuppressing macrolide is tacrolimus, pinecrolimus or sirolimus.

40. A method of treating a muscular dystrophy in a human subject in need thereof comprising administering a recombinant adeno-virus associated (rAAV) selected from the group consisting of: AAVrh.74.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05; and an immunosuppressing regimen, wherein the immunosuppressing regimen comprises the steps of
i) orally administering an anti-inflammatory steroid about 24 hours prior to administration of the rAAV, and administering an anti-inflammatory steroid at least once a day from day 1 to 30 days after administration of the rAAV or from day 1 to 60 days after administration of the rAAV,
ii) intravenously administering an anti-CD20 antibody about 14 days prior to administration of the rAAV, about 7 days prior to administration of the rAAV and within about 24 hours of the administration of the rAAV, and optionally administering the anti-CD20 antibody after administration of the rAAV,
iii) orally administering an immunosuppressing macrolide at least once a day for at least three days prior to administration of the rAAV, and optionally administering the an immunosuppressing macrolide after administration of the rAAV.

41-46. (canceled)

47. A method of treating a muscular dystrophy in a human subject in need thereof comprising subjecting the subject's plasma to at least one therapeutic plasma exchange (TPE) prior to administration of a second dose of recombinant adeno-virus associated (rAAV) selected from the group consisting of: AAVrh.74.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05, wherein the subject was administered a first dose of rAAV prior to being subjected to TPE.

48. A method of treating a muscular dystrophy in a human subject in need thereof comprising the steps of
a) administering a first dose of recombinant adeno-virus associated selected from the group consisting of: AAVrh.74.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, and rAAVrh.74.MHCK7.huAN05;
b) subjecting the subject's plasma to at least one therapeutic plasma exchange (TPE), and
c) administering a second dose or rAAV.

49. (canceled)

50. (canceled)

51. The method of claim 48, wherein the subject's plasmas is subject to at least two TPE or at least three TPE prior to administration of the 2^{nd} dose or rAAV.

52. A method of treating muscular dystrophy in a human subject in need thereof comprising the steps of
a) subjecting the subject's plasma to at least one therapeutic plasma exchange (TPE) prior to administering recombinant adeno-virus associated (rAAV)
b) administering rAAV, and
wherein the rAAV is rAAV.MHCK7.microdystrophin, AAVrh.74.tMCK.CAPN3, rAAVrh.74.MHCK7.DYSF, scAAVrh.74.MHCK7.hSGCG, AAVrh74.tMCK.hSCGA, scAAVrh74.MHCK7.HSGCB, or rAAVrh.74.MHCK7.huAN05.

53. The method of claim 52 wherein the subject's plasma is subjected to at least two TPE, at least three TPE, at least four TPE, at least five TPE rAAV, at least six TPE or at least seven TPE prior to administering.

54. The method of claim 52 wherein the subject's plasma is subjected to TPE for at least 9 days prior to administration of the rAAV, at least 7 days prior to administration of the rAAV, 5 days prior to administration of the rAAV, 2 days prior to administration of the rAAV or on the day the rAAV is administered.

55. (canceled)

56. (canceled)

57. The method of claim 52, wherein the subject is administered an anti-inflammatory steroid about 24 hours prior to administration of the rAAV, or at least once a day from day 1 to 60 days after administration of the rAAV.

58-61. (canceled)

62. The method of claim 21 wherein the rAAV is administered by systemic route of administration.

63. The method of claim 62 wherein the systemic route of administration is an intravenous route and the dose of the rAAV administered is about 2×10¹⁴ vg/kg.

64-68. (canceled)

69. The method of claim 21 wherein the rAAV comprises
(a) the human micro-dystrophin nucleotide sequence of SEQ ID NO: 1,
(b) the MHCK7 promoter sequence of SEQ ID NO: 2 or SEQ ID NO:7,
(c) the nucleotide sequence of SEQ ID NO: 44, or
(d) AAVrh74.MHCK7.micro-dystrophin construct nucleotide sequence of SEQ ID NO: 9 or nucleotides 55-5021 of SEQ ID NO: 3.

70-72. (canceled)

73. The method of claim 21 wherein the human subject is suffering from Duchenne muscular dystrophy, and the rAAV is administered by intravenous infusion over approximately one hour at a dose of about 2×10¹⁴ vg/kg, and wherein the rAAV comprises the AAVrh74.MHCK7.micro-dystrophin construct nucleotide sequence of SEQ ID NO: 9 or of nucleotides 55-5021 of SEQ ID NO: 3.

74. (canceled)

75. The method of claim 21 wherein the human subject is suffering from LGMD2E, and the rAAV is administered by intravenous infusion at a dose of about 2×10¹⁴ vg/kg, and wherein the rAAV comprises the rAAV is scAAVrh74.MHCK7.HSGCB comprising the nucleotide sequence of SEQ ID NO: 44.

76. The method of claim 21 wherein the level of micro-dystrophin gene expression in a cell of the subject is increased after administration of the rAAV as compared to the level of micro-dystrophin gene expression before administration of the rAAV.

77. The method of claim 21, further comprising the step of determining the presence of anti-AAVrh.74 antibodies in serum or plasma of said subject.

78. The method of claim 77, wherein the determining step is performed prior to the step of administering said administering of an immunosuppressing regimen.

79. The method of claim 78 wherein the determining step is performed prior to any administration of an AAV to said subject.

80. The method of claim 79 where the determining step is performed prior to administration of aAAVrh.74 to said subject.

81. (canceled)

82. The method of claim 79, further comprising a step of comparing the level of anti-AAVrh.74 antibodies in serum or plasma of said subject to a positive control.

83. The method of claim 82 wherein said positive control utilizes an anti-AAVrh.74 monoclonal antibody.

84. The method of claim 80, wherein the determining step comprises utilizing an anti-AAVrh.74 monoclonal antibody.

85. The method of claim 84, wherein said determination step comprises utilizing an immunofluorescence assay, an immunohistochemical assay, a Western blot, a direct enzyme-linked immunosorbent assay (ELISA), an indirect ELISA, a sandwich ELISA, a competitive ELISA, a reverse ELISA, a chemiluminescence assay, a radioimmunoassay, or an immunoprecipitation assay.

86. The method of claim 80, wherein said monoclonal antibody comprises a VH CDR1 amino acid sequence selected from the group consisting of NYGMN (SEQ ID NO: 20), DYGMN (SEQ ID NO: 22), YTFTNYGMN (SEQ ID NO: 21), and YTFTKYGMN (SEQ ID NO: 23).

87. The method of claim 80, wherein said monoclonal antibody comprises a VH CDR2 amino acid sequence selected from the group consisting of WINTYTGEPTYADDFKG (SEQ ID NO: 24), WINTNTGEPTYGDDFKG (SEQ ID NO: 25), and WMGWINTYTGEPTY (SEQ ID NO: 26).

88. The method of claim 80, wherein said monoclonal antibody comprises a VH CDR3 amino acid sequence selected from the group consisting of GVAHYSDSRFAFDY (SEQ ID NO: 27), GNAHPGGSAFVY (SEQ ID NO: 28), RGSYYYDSSPAWFAY (SEQ ID NO: 29), RGVDSSGYGAFAY (SEQ ID NO: 30), and TRGTSTMISTFAFVY (SEQ ID NO: 31).

89. The method of claim 80, wherein said monoclonal antibody comprises a VL CDR1 amino acid sequence selected from the group consisting of SVSSSVSYMH (SEQ ID NO: 32), SASSGVTYMH (SEQ ID NO: 33), SSVSYMH (SEQ ID NO: 34), and SSVRYMH (SEQ ID NO: 35).

90. The method of claim 80, wherein said monoclonal antibody comprises a VL CDR2 amino acid sequence selected from the group consisting of YTSNLAS (SEQ ID NO: 36), RTSNLAS (SEQ ID NO: 37), LWIYSTSNLAS (SEQ ID NO: 38), and VWIYSTSNLAS (SEQ ID NO: 39).

91. The method of claim 80, wherein said monoclonal antibody comprises a VH CDR3 amino acid sequence selected from the group consisting of QQRSSYPFT (SEQ ID NO: 40), QQRSTYPF (SEQ ID NO: 41), QQRSFYPF (SEQ ID NO: 42), and QQRTYYPF (SEQ ID NO: 43).

92. The method of claim 80, wherein said monoclonal antibody comprises
(a) a variable heavy chain (VH) sequence set forth in SEQ ID NO: 10, 12, 14, 16, or 18,
(b) a variable light chain (VL) sequence set forth in SEQ ID NO: 11, 13, 15, 17, or 19
(c) a variable heavy chain (VH) sequence set forth in SEQ ID NO: 10, 12, 14, 16, or 18, and a variable light chain (VL) sequence set forth in SEQ ID NO: 11, 13, 15, 17, or 19.

93. (canceled)

94. (canceled)

95. The method of claim 80, wherein said determination is quantitative, wherein said subject is identified as seropositive for anti-AAVrh.74 antibodies based said quantitation, and wherein said immunosuppressing regimen or TPE is selectively administered to the seropositive subject.
